SEQUENCE LISTING (58 sequences)

SEQ ID NO: 1 - DNA, 24 bp, Artificial Sequence
  Forward Primer for MageA2B-QU
gttgtgaatttagggaagttatgg

SEQ ID NO: 2 - DNA, 24 bp, Artificial Sequence
  Reverse Primer for MageA2B-QU
catcaaaccattactcaaaacaaa

SEQ ID NO: 3 - DNA, 28 bp, Artificial Sequence
  Forward Primer for MageA3-QU
attattttattttgttgatttttgttgt

SEQ ID NO: 4 - DNA, 24 bp, Artificial Sequence
  Reverse primer for MageA3-QU
ctcaaaactcacatacctaaccac

SEQ ID NO: 5 - DNA, 23 bp, Artificial Sequence
  Forward Primer for MageA4-QU
taaaaatttgttttttttattga

SEQ ID NO: 6 - DNA, 26 bp, Artificial Sequence
  Reverse Primer for MageA4-QU
ataatacaaaattatttaaaatcatc

SEQ ID NO: 7 - DNA, 25 bp, Artificial Sequence
  Forward Primer for Magea11-QU
agtagatattattggtagaggatgg

SEQ ID NO: 8 - DNA, 25 bp, Artificial Sequence
  Reverse Primer for Magea11-QU
cttccctaaatttacaacaaaaacg

SEQ ID NO: 9 - DNA, 25 bp, Artificial Sequence
  Forward primer for GPR17-QU
ttatttcgtgagtaagatttttcgg

SEQ ID NO: 10 - DNA, 24 bp, Artificial Sequence
  Reverse Primer for GPR17-QU
gactatcataacaacaaacgacgc

SEQ ID NO: 11 - DNA, 25 bp, Artificial Sequence
  Forward primer for GRIN1-QU
ttttttaagttagggaggggtatgt

SEQ ID NO: 12 - DNA, 25 bp, Artificial Sequence
  Reverse primer for GRIN1-QU
aaacaccaaaaatcactaaaacacc

SEQ ID NO: 13 - DNA, 23 bp, Artificial Sequence
  Forward primer for H19-QU
tttagagtttgtggttaaggtgg

SEQ ID NO: 14 - DNA, 28 bp, Artificial Sequence
  Reverse primer for H19-QU
aacaaaaaaatcatataaaaaaaacata

SEQ ID NO: 15 - DNA, 23 bp, Artificial Sequence
  Forward primer for C19ORG28-QU
tgtatattgtttgtgggttatga

SEQ ID NO: 16 - DNA, 25 bp, Artificial Sequence
  Reverse primer for C19ORF28-QU
caaaatctcaaaaaaactacccaac

SEQ ID NO: 17 - DNA, 29 bp, Artificial Sequence
  Forward primer TKTL1-QU
tgtgtagagaaagaagattttgtatttgt

SEQ ID NO: 18 - DNA, 25 bp, Artificial Sequence
  Reverse primer for TKTL1-QU
cccaatacatttatataatatcaat

SEQ ID NO: 19 - DNA, 20 bp, Artificial Sequence
  Forward primer for magea2
atctgcctgtgggtcttcat

SEQ ID NO: 20 - DNA, 20 bp, Artificial Sequence
  Reverse primer for magea2
gaagaggaagaagcggtctg

SEQ ID NO: 21 - DNA, 20 bp, Artificial Sequence
  Forward primer for magea3
aagatctgccagtgggtctc

SEQ ID NO: 22 - DNA, 20 bp, Artificial Sequence
  Reverse primer for magea3
ccccagggtgacttcaacta

SEQ ID NO: 23 - DNA, 20 bp, Artificial Sequence
  Forward primer for magea4
aggagaagatctgcctgtgg

SEQ ID NO: 24 - DNA, 20 bp, Artificial Sequence
  Reverse primer for magea4
cagcctcctgctcctcagta

SEQ ID NO: 25 - DNA, 20 bp, Artificial Sequence
  Forward primer for boris
cacattcgtacccacactgg

SEQ ID NO: 26 - DNA, 20 bp, Artificial Sequence
  Reverse primer for boris
tcccctgatccacacttctc

SEQ ID NO: 27 - DNA, 20 bp, Artificial Sequence
  Forward primer for grin1
gtcctccaaagacacgagca

SEQ ID NO: 28 - DNA, 20 bp, Artificial Sequence
  Reverse primer for grin1
ccgtctgtctgtctgtctgc

SEQ ID NO: 29 - DNA, 19 bp, Artificial Sequence
  Forward primer for tktl1
gatcactacccgcaaggtg

SEQ ID NO: 30 - DNA, 20 bp, Artificial Sequence
  Reverse primer for tktl1
cacagggtaaagaggccaaa

SEQ ID NO: 31 - DNA, 20 bp, Artificial Sequence
  Forward primer for h19
gagctctcaggagggaggat

SEQ ID NO: 32 - DNA, 18 bp, Artificial Sequence
  Reverse primer for h19
agacctggcctcgtctcc

SEQ ID NO: 33 - DNA, 18 bp, Artificial Sequence
  Forward primer for c19orf28
acccttgcccctcagagc

SEQ ID NO: 34 - DNA, 18 bp, Artificial Sequence
  Reverse primer for c19orf28
ggccacagcaggaggcta

SEQ ID NO: 35 - DNA, 21 bp, Artificial Sequence
  Forward primer for gpr17
aactctcaggctctgactcca

SEQ ID NO: 36 - DNA, 20 bp, Artificial Sequence
  Reverse primer for gpr17
gccagggtattgccaactaa

SEQ ID NO: 37 - DNA, 20 bp, Artificial Sequence
  Forward primer for magea11
gaggagaacaagtgctgtgg

SEQ ID NO: 38 - DNA, 20 bp, Artificial Sequence
  Reverse primer for magea11
agcagcaggcaactcctcta

SEQ ID NO: 39 - DNA, 26 bp, Artificial Sequence
  Primer Name quMAGEA3F, designed to not contain CG dinucleotides.
ttgtgagatttttgttttgagtaatg

SEQ ID NO: 40 - DNA, 24 bp, Artificial Sequence
  Primer Name quMAGEA3R, designed to not contain CG dinucleotides.
caacctaaaaatcttcccctacaa

SEQ ID NO: 41 - DNA, 25 bp, Artificial Sequence
  Primer Name quMAGEA12F, designed to not contain CG dinucleotides.
ttttgttttgagtaatggtttgatg

SEQ ID NO: 42 - DNA, 25 bp, Artificial Sequence
  Primer Name quMAGEA12R, designed to not contain CG dinucleotides.
ccctctatctaaaataaaacccacc

SEQ ID NO: 43 - DNA, 30 bp, Artificial Sequence
  Primer Name quMAGEA4F, designed to not contain CG dinucleotides.
atttatatttttatttaggtaggatttttg

SEQ ID NO: 44 - DNA, 23 bp, Artificial Sequence
  Primer Name quMAGEA4R, designed to not contain CG dinucleotides.
tctcaaactacaaatcaaacaca

SEQ ID NO: 45 - DNA, 25 bp, Artificial Sequence
  Primer Name quMAGEA1F, designed to not contain CG dinucleotides.
tttggtttttgttaggaaatatttg

SEQ ID NO: 46 - DNA, 25 bp, Artificial Sequence
  Primer Name quMAGEA1R, designed to not contain CG dinucleotides.
acaaaacctaaatcaaattccttca

SEQ ID NO: 47 - DNA, 26 bp, Artificial Sequence
  Primer Name quSBSN_F, designed to not contain CF dinucleotides.
taagatgtgattggtttagatgttga

SEQ ID NO: 48 - DNA, 22 bp, Artificial Sequence
  Primer Name quSBSN_R, designed to not contain CF dinucleotides.
cctacaacctaccatacccact

SEQ ID NO: 49 - DNA, 30 bp, Artificial Sequence
  Primer Name quTKTL1F, designed to not contain CF dinucleotides.
taaatattttagattttttgatttgtttgt

SEQ ID NO: 50 - DNA, 25 bp, Artificial Sequence
  Primer Name quTKTL1R, designed to not contain CF dinucleotides.
taatatcaatacaactccctccaca

SEQ ID NO: 51 - DNA, 25 bp, Artificial Sequence
  Primer Name quMAGEA5F, designed to not contain CF dinucleotides.
tggttttgtttttgtttttaattga

SEQ ID NO: 52 - DNA, 25 bp, Artificial Sequence
  Primer Name quMAGEA5R, designed to not contain CF dinucleotides.
aaataacccaaactaaaaaatccac

SEQ ID NO: 53 - DNA, 29 bp, Artificial Sequence
  Primer Name quZNF711F, designed to not contain CF dinucleotides.
tttttattttgtagtaagggtagtgtgat

SEQ ID NO: 54 - DNA, 23 bp, Artificial Sequence
  Primer Name quZNF711R, designed to not contain CF dinucleotides.
cccttaaaatcctcacattcaaa

SEQ ID NO: 55 - DNA, 25 bp, Artificial Sequence
  Primer Name quNYESO1F, designed to not contain CF dinucleotides.
ggtattgtggttattttttggtttg

SEQ ID NO: 56 - DNA, 23 bp, Artificial Sequence
  Primer Name quNYESO1R, designed to not contain CF dinucleotides.
cacacaaaaaccctacttccaac

SEQ ID NO: 57 - DNA, 25 bp, Artificial Sequence
  Primer Name quG6PD_F, designed to not contain CF dinucleotides.
gttttggagagaagtttgagtttgt

SEQ ID NO: 58 - DNA, 27 bp, Artificial Sequence
  Primer Name quG6PD-R, designed to not contain CF dinucleotides.
tccaaaaaacaaaaaaacatataaaca

(19) United States
(12) Patent Application Publication
(10) Pub. No.: US 2012/0142546 A1
(43) Pub. Date: Jun. 7, 2012

(54) HYPOMETHYLATED GENES IN CANCER

(75) Inventors: Joseph A. Califano, Owings Mills, MD (US); Ian M. Smith, Baltimore, MD (US)

(73) Assignee: THE JOHNS HOPKINS UNIVERSITY, Baltimore, MD (US)

(21) Appl. No.: 12/747,304

(22) PCT Filed: Dec. 10, 2008
(86) PCT No.: PCT/US08/86294

§ 371 Date: Jan. 12, 2012

Related U.S. Application Data
(62) Division of application No. 61/007,025, filed on Dec. 10, 2007.

(51) Int. Cl. C12Q 1/68; C40B 30/04

(52) U.S. Cl. 506/9; 435/6.11

(57) ABSTRACT

The present invention provides methods and kits for identifying a cell that exhibits or is predisposed to exhibiting unregulated growth by detecting hypomethylation of a gene or a regulatory region in at least one gene in the cell. Also provided are methods for diagnosis or prognosis of a proliferative disorder in a subject. Also provided are methods of ameliorating a cell proliferative disorder in a subject by administering to the subject an agent that methylates a hypomethylated gene or regulatory region thereof. In some aspects, the gene or regulatory region thereof is TKTL1, H19, MAGEA2, MAGEA3, MAGEA4, MAGEA11, GPR1 7, GRTN1, C19ORF28, MAGEA12, MAGEA1, MAGEA5, NY-ESO-1, MAGEA9, MAGEA6, MAGEB2, CT45-2, SBSN, G6PD, ZNF711, CrispL, KRT86, KIPV467, KRT81, CSPG5, PP1R14A, KISS1R, KIAA1937 protein, SOX30, DEAD, or KBGP.

### BACKGROUND OF THE INVENTION

1. Field of the Invention

The present invention relates generally to methods for detecting the presence of or risk of developing cancer and more specifically to methods for detecting the presence of hypomethylation in various genes or regulatory regions thereof.

2. Background Information

Epigenetic alterations in promoter methylation and histone acetylation have been associated with cancer-specific expression differences in human malignancies, including for example, head and neck cancer and non-small cell lung carcinoma (NSCLC). Methylation has been primarily considered as a mechanism of tumor suppressor gene (TSG) inactivation, and comprehensive whole-genome profiling approaches to promoter hypermethylation have identified multiple novel putative TSGs silenced by promoter hypermethylation.

Indirect evidence supports a role for hypomethylation in tumor development. Global genomic hypomethylation has been reported in almost all solid tumors. Mice with functional disruption of DNA methyltransferase 1 (DNMT1) function demonstrate significant genomic hypomethylation in all tissues and develop aggressive T-cell lymphomas with chromosomal instability. In solid human tumors, meta-analysis shows an overall correlation between global hypomethylation and advanced tumor stage.

To date, only sporadic examples of promoter hypomethylation associated with unmasked expression of putative oncogenes have been reported, including: R-Ras in gastric cancer, c-Neu in transgenic mouse models, the Hox11 proto-oncogene in leukemia, BCL-2 gene hypomethylation and high-level expression in B-cell chronic lymphocytic lymphomas, demethylation in MMTV/N-rasN transgenic mice, and rare activation of two RAS family members in colon cancer and small cell lung cancer. These observations demonstrate that proto-oncogenes with tissue-specific or developmentally restricted expression—i.e., during early growth, differentiation, or gametogenesis—may be inappropriately re-expressed in cancers via epigenetic alteration, including demethylation.

Cancer/testis antigens (CTAs) have been shown to be overexpressed in various tumor types, with little or no expression in normal human tissue; however, the mechanism of this differential expression is not well understood. It is also known that CTAs, especially those encoded by the X chromosome (CT-X antigens), are expressed in association with promoter demethylation or whole genomic hypomethylation. To date, a comprehensive, genome-wide approach to identify coordinately expressed CTAs and other differentially expressed genes activated by promoter demethylation in NSCLC has not been conducted.

### SUMMARY OF THE INVENTION

The present invention is based on the discovery that some genes have promoters that are demethylated and transcriptionally upregulated in cancer. This discovery is useful for cancer screening, risk-assessment, prognosis, and identification of subjects responsive to a therapeutic regimen. Accordingly, there are provided methods for detecting a cellular proliferative disorder in a subject. The subject may have or be at risk of having a cellular proliferative disorder. The method of the invention is useful for diagnostic as well as prognostic analyses.

In one embodiment of the invention, there are provided methods for identifying a cell that exhibits or is predisposed to exhibiting unregulated growth. The method includes detecting hypomethylation of a gene or a regulatory region in at least one gene in the cell, wherein the at least one gene is hypomethylated as compared to a corresponding normal cell not exhibiting unregulated growth, thereby identifying the cell as exhibiting or predisposed to exhibiting unregulated growth. In some aspects, at least two genes or regulatory regions are hypomethylated and the at least two genes are coordinately expressed in the cell undergoing unregulated cell growth. In particular embodiments, the regulatory region of the at least one gene comprises a BORIS binding site. In certain embodiments, the regulatory region of the at least one gene includes a promoter of a gene selected from the group consisting of TKTL1, H19, MAGEA2, MAGEA3, MAGEA4, MAGEA11, GPR17, GRIN1, C19ORF28, MAGEA12, MAGEA1, MAGEA5, NY-ESO-1, MAGEA9, MAGEA6, MAGEB2, CT45-2, SBSN, G6PD, ZNF711, CrispL (cyteine-rich secretory protein LCCL domain), KRT86 (keratin, hair, basic, 6 monilethrix), KIPV467, KRT81 (keratin, hair, basic, 1), CSPG5 (chondroitin sulfate proteoglycan 5), PP1R14A (protein phosphatase 1, regulatory inhibitor 14), KISS1R (G protein coupled receptor 54), KIAA1937 protein, SOX30 (SRY sex determining region Y-box 30), DEAD (Asp-Glu-Ala-Asp box polypeptide), and KBGP (Kell Blood group precursor McLeod phenomenon).

In another embodiment, there are provided methods for diagnosing a disorder in a subject having or at risk of developing a cell proliferative disorder. The method includes contacting a nucleic acid-containing sample from cells of the subject with an agent that provides a determination of the methylation state of at least one regulatory region of a gene, wherein the at least one regulatory region is hypomethylated in a cell undergoing unregulated cell growth as compared to a corresponding normal cell; and identifying hypomethylation of the regulatory region in the nucleic acid-containing sample, as compared to the same region of the at least one regulatory region in a subject not having the proliferative disorder, wherein hypomethylation is indicative of a subject having or at risk of developing the proliferative disorder. In some aspects, at least two genes or regulatory regions are hypomethylated and the at least two genes are coordinately expressed in the cell undergoing unregulated cell growth. In particular embodiments, the regulatory region of the at least one gene comprises a BORIS binding site. In certain embodiments, the regulatory region of the at least one gene includes a promoter of a gene selected from the group consisting of TKTL1, H19, MAGEA2, MAGEA3, MAGEA4, MAGEA11, GPR17, GRIN1, C19ORF28, MAGEA12, MAGEA1, MAGEA5, NY-ESO-1, MAGEA9, MAGEA6, MAGEB2, CT45-2, SBSN, G6PD, ZNF711, CrispL, KRT86, KIPV467, KRT81, CSPG5, PP1R14A, KISS1R, KIAA1937 protein, SOX30, DEAD, and KBGP.

In yet another embodiment of the invention, there are provided methods of determining the prognosis of a subject having a cell proliferative disorder. The method includes determining the methylation state of at least one regulatory region of a gene in a nucleic acid sample from the subject, wherein hypomethylation as compared to a corresponding normal cell in the subject or a subject not having the disorder, is indicative of a poor prognosis. In some aspects, at least two genes or regulatory regions are hypomethylated and the at least two genes are coordinately expressed in the cell undergoing unregulated cell growth. In particular embodiments, the regulatory region of the at least one gene comprises a BORIS binding site. In certain embodiments, the regulatory region of the at least one gene includes a promoter of a gene selected from the group consisting of TKTL1, H19, MAGEA2, MAGEA3, MAGEA4, MAGEA11, GPR17, GRIN1, C19ORF28, MAGEA12, MAGEA1, MAGEA5, NY-ESO-1, MAGEA9, MAGEA6, MAGEB2, CT45-2, SBSN, G6PD, ZNF711, CrispL, KRT86, KIPV467, KRT81, CSPG5, PP1R14A, KISS1R, KIAA1937 protein, SOX30, DEAD, and KBGP.

In still another embodiment of the present invention, there are provided methods of ameliorating a cell proliferative disorder in a subject in need thereof. The method includes administering to the subject an agent that methylates at least one regulatory region in a gene that is demethylated as compared to a subject not having the disorder, thereby reducing expression of the at least one gene and ameliorating the cell proliferative disorder. In some aspects, at least two genes or regulatory regions are hypomethylated and the at least two genes are coordinately expressed in the cell undergoing unregulated cell growth. In particular embodiments, the regulatory region of the at least one gene comprises a BORIS binding site. In certain embodiments, the regulatory region of the at least one gene includes a promoter of a gene selected from the group consisting of TKTL1, H19, MAGEA2, MAGEA3, MAGEA4, MAGEA11, GPR17, GRIN1, C19ORF28, MAGEA12, MAGEA1, MAGEA5, NY-ESO-1, MAGEA9, MAGEA6, MAGEB2, CT45-2, SBSN, G6PD, ZNF711, CrispL, KRT86, KIPV467, KRT81, CSPG5, PP1R14A, KISS1R, KIAA1937 protein, SOX30, DEAD, and KBGP.

In a further embodiment of the present invention, there are provided methods of identifying a gene activated by hypomethylation. The method includes comparing an expression analysis of a cell treated with an agent that reduces methylation to an expression analysis of a control cell not treated with the agent, wherein an increase in expression of a gene is indicative of a gene activated by demethylation. Certain embodiments may further include an expression analysis of a tissue sample and a tumor sample from the same tissue of origin as the normal cell, wherein an increase in expression of a gene in a tumor sample as compared to a normal sample is correlated to the genes activated by demethylation in the treated cell.

In another embodiment of the present invention, there are provided methods for determining whether a subject is responsive to a particular therapeutic regimen. The method includes determining the methylation status of one or more genes or regulatory regions thereof, selected from the group consisting of TKTL1, H19, MAGEA2, MAGEA3, MAGEA4, MAGEA11, GPR17, GRIN1, C19ORF28, MAGEA12, MAGEA1, MAGEA5, NY-ESO-1, MAGEA9, MAGEA6, MAGEB2, CT45-2, SBSN, G6PD, ZNF711, CrispL, KRT86, KIPV467, KRT81, CSPG5, PP1R14A, KISS1R, KIAA1937 protein, SOX30, DEAD, and KBGP, wherein hypomethylation of the gene or regulatory region thereof as compared with a normal subject is indicative of a subject who is responsive to the therapeutic regimen. In certain embodiments, therapeutic regimen is administration of a chemotherapeutic agent. In other embodiments, the therapeutic regimen is administration of a vaccine directed to a protein encoded by the hypomethylated gene.

In another embodiment, the invention provides a kit useful for the detection of a methylated CpG-containing nucleic acid in determining the methylation status of one or more genes or regulatory regions thereof, selected from the group consisting of TKTL1, H19, MAGEA2, MAGEA3, MAGEA4, MAGEA11, GPR17, GRIN1, C19ORF28, MAGEA12, MAGEA1, MAGEA5, NY-ESO-1, MAGEA9, MAGEA6, MAGEB2, CT45-2, SBSN, G6PD, ZNF711, CrispL, KRT86, KIPV467, KRT81, CSPG5, PP1R14A, KISS1R, KIAA1937 protein, SOX30, DEAD, and KBGP. The kit includes carrier element containing one or more containers having a first container containing a reagent which modifies unmethylated cytosine and a second container containing primers for amplification of the one or more genes or regulatory regions thereof, wherein the primers distinguish between modified methylated and nonmethylated nucleic acid.

### BRIEF DESCRIPTION OF THE DRAWINGS

FIG. 1A provides a schematic of the integrative epigenetic screening strategy employed in Example 1. FIG. 1B shows a representative COPA graph of MAGEA3 demonstrating the statistical approach to finding candidate overexpressed oncogenes. FIG. 1C shows a plot of the upregulation of expression of the target genes after treatment with 5-aza/TSA in cell lines as measured by QRT-PCR.

FIG. 2 depicts the promoter methylation status in primary tissues. FIG. 2A shows the bisulfite sequencing results in 10 tumors and 10 normals for: TKTL1 (4/10, p<0.05), H19 (6/10, p<0.05), MAGEA2 (5/10, p<0.05), MAGEA3 (5/10, p<0.05), MAGEA4 (5/10, p<0.05), MAGEA11 (5/10, p<0.05), GPR17 (3/10, p<0.10), GRIN1 (6/10, p<0.05), C19ORF28 (5/10, p<0.05). FIGS. 2B-J show the QRT-PCR expression with the bisulfite sequencing of the respective promoter below (white is unmethylated, grey is methylated).

FIG. 3 shows depicts the results of the transient transfection of target genes in minimally transformed oral keratinocytes. FIG. 3A-D show plots of the transient transfection of an H19 construct, a MAGEA2 construct, a TKTL1 construct, and a MAGEA4 construct into OKF6-Tert-1R cells, for A-D, respectively. FIG. 3E shows plots of the QUMSP percentage of C19ORF28, GRIN1, H19, MAGEA11, MAGEA2, MAGEA3, GPR17, and TKTL1 conducted in a separate cohort of head and neck cancer patients using 25 tumors and 11 upper aerodigestive mucosal samples to assay promoter demethylation. FIG. 3F shows the QUMSP results for an independent cohort of 14 lung normals and 13 lung tumor patients. Significant differences in QUMSP were found in H19, MAGEA11, MAGEA2, and MAGEA3.

FIG. 4 shows plots demonstrating TKTL1 forced overexpression via transient transfection in background low expressing JHU-011 cells (FIG. 4A); induces increased anchorage dependent colony formation and TKTL1 shRNA in high-expressing FaDu cell line (FIG. 4B); induces growth inhibition. Anchorage independent growth of UM-22B cells is significantly inhibited by TKTL1 shRNA (FIG. 4C); with decrease in colony size (FIG. 4D).

FIG. 5 shows plots depicting overexpression and demethylation in human cancers (non small cell lung cancer, lymphoma, melanoma, pancreatic cancer, and urothelial cancer). FIG. 5A shows the expression of H19 in these cancers. FIG. 5B shows MAGEA2 expression, FIG. 5C shows TKTL1 expression, and FIG. 5D shows MAGEA4.

FIG. 6 shows gene expression and demethylation correlation. FIG. 6A shows the gene expression correlation p-value matrix for the coexpression for each gene pair across all tumors. This comparison shows the correlation of each gene pair in 49 head and neck tumors. FIG. 6B shows the gene pair expression p-value correlation matrix for 80 NSCLC. FIG. 6C shows a phylogram of the promoters of interest based on ClustalW analysis after multiple sequence alignment. The region of significant homology is shown after sequence alignment. FIG. 6D shows the promoter hypomethylation (QUMSP) correlation p-value matrix for HNSCC (25 tumors). FIG. 6E shows the promoter hypomethylation (QUMSP) correlation p-value matrix for NSCLC (13 tumors).

FIG. 7A shows a correlation of BORIS expression with expression of target genes in HNSCC (QRT-PR) heat map (Pearson correlation). FIG. 7B shows plots of the growth of cells following the transient transfection of BORIS construct into NIH-3T3 and 01(F6-Tert1R cell lines. FIG. 7C shows a plot of the anchorage independent growth assayed after transfection with empty vector (EV), CTCF, and BORIS at various concentrations of doxycycline, with a representative colony shown. FIG. 7D shows a plot of QUMSP of nine targets of interest after transfection with empty vector (untreated) and BORIS construct (treated) in presence of 0.0625 ug/ml of doxycycline. FIG. 7E shows a plot of the fold increase in expression measured by quantitative RT-PCR of nine targets of interest after BORIS transfection.

FIG. 8 shows a plot of the fold upregulation of mRNA expression in treated minimally-transformed cell lines measured by Affymetrix U133 Plus 2.0.

FIG. 9 shows BORIS correlates with gene expression in all cancers (using the expO cohort of 1041 human cancers of various tumor sites and histologies). Shown are microarray median-normalized expression of the targets compared to BORIS expression in 1041 human cancers

FIG. 10A shows the integrative epigenetic screening strategy employed in Example 2. FIG. 10B shows a plot of the mRNA expression in treated normal lung cell lines, NHBE and SAEC. FIG. 10C shows a representative COPA graph of MAGEA12 demonstrating the statistical approach used to find candidate overexpressed CTAs and related genes.

FIG. 11 shows the bisulfite sequencing results with associated p values in 28 NSCLC tumor samples and 11 normal lung tissues for the indicated genes. Shaded boxes represent methylated promoters, “ND”=methylation status not determined by bisulfite sequencing.

FIG. 12A shows a plot of QUMSP conducted in a cohort of 28 NSCLC and 11 normal lung tissues. FIG. 12B shows the promoter hypomethylation (QUMSP) correlation p-value matrix for NSCLC (n=28; Spearman's correlation permutation test).

FIG. 13A shows a heat map of transcript expression as measured by the Affymetrix Human Genome. U133 Plus 2.0 mRNA expression platform for 40 normal lung samples from non-cancer patients and 111 NSCLC primary tissue samples. FIG. 13B shows the Pearson's correlation coefficient p-value matrix for gene expression which tests for the coexpression of each gene pair across all tumors. This comparison shows the expression correlation of each gene pair in 111 NSCLC. Values to the upper right have been corrected with the Benjamin Hochberg multiple test correction to decrease the false discovery rate; uncorrected values are displayed in the lower left. Shaded cells represent significant p-values.

FIG. 14 shows a plot of the relative fold upregulation of expression of each gene after treatment with 5-aza/TSA is shown in NHBE and SAEC cell lines as measured by quantitative RT-PCR.

FIG. 15 depicts the target gene expression is upregulated in NSCLC vs. normal lung tissues. FIGS. 15A-J show the quantitative RT-PCR in a cohort of 28 NSCLC and 5 normal lung tissues for each gene.

FIG. 16A-D shows scatter plots showing Log 2 QRT-PCR values plotted against Log 2 QUMSP for 28 NSCLC and 5 normal lung tissues for MAGEA12 (p=0.024), MAGEA4 (p<0.004), SBSN (p=0.004) and NY-ESO-1 (p<0.004).

FIG. 17 shows a table of containing a ranking of the 290 significant genes found after combing the three rank ordered lists (“-”=Not determined).

### DETAILED DESCRIPTION OF THE INVENTION

The present invention is based on the discovery that several genes have promoters that are demethylated and transcriptionally upregulated in cancer. Accordingly, in a first embodiment of the invention, there are provided methods for identifying a cell that exhibits or is predisposed to exhibiting unregulated growth. The method includes detecting hypomethylation of a gene or a regulatory region in at least one gene in the cell, wherein the at least one gene is hypomethylated as compared to a corresponding normal cell not exhibiting unregulated growth, thereby identifying the cell as exhibiting or predisposed to exhibiting unregulated growth.

The genes or regulatory regions thereof whose methylation status is detected in the methods provided herein can be any gene or regulatory region thereof identified as hypomethylated in a cell exhibiting unregulated growth as compared to a corresponding normal cell, not undergoing unregulated cell growth. In certain embodiments, at least two genes or regulatory regions are hypomethylated and the at least two genes are coordinately expressed in the cell undergoing unregulated cell growth. In other aspects, at least three, or at least four, or at least five, or more genes or regulatory regions are hypomethylated.

In certain embodiments, the gene or regulatory region is one or more of the genes identified herein (the “target genes”). In particular embodiments, the gene or regulatory region thereof is selected from the group consisting of TKTL1, H19, MAGEA2, MAGEA3, MAGEA4, MAGEA11, GPR17, GRIN1, C19ORF28, MAGEA12, MAGEA1, MAGEA5, NY-ESO-1, MAGEA9, MAGEA6, MAGEB2, CT45-2, SBSN, G6PD, ZNF711, CrispL, KRT86, KIPV467, KRT81, CSPG5, PP1R14A, KISS1R, KIAA1937 protein, SOX30, DEAD, and KBGP. In some embodiments, the gene or regulatory region thereof is one or more of TKTL1, H19, MAGEA2, MAGEA3, MAGEA4, MAGEA11, GPR17, GRIN1, C19ORF28, MAGEA12, MAGEA1, MAGEA5, NY-ESO-1, MAGEA9, MAGEA6, MAGEB2, CT45-2, SBSN, G6PD, and ZNF711. In certain embodiments, the gene or regulatory region thereof is one or more of TKTL1, H19, MAGEA2, MAGEA3, MAGEA4, MAGEA11, GPR17, GRIN1, and C19ORF28. In other embodiments the gene or regulatory region thereof is one or more of MAGEA3, MAGEA12, MAGEA4, MAGEA1, MAGEA5, NY-ESO-1, MAGEA9, MAGEA6, MAGEB2, and CT45-2.

As provided herein, hypomethylation may occur in the gene or regulatory region thereof. In some embodiments, the hypomethylation occurs within the regulatory region of the genes identified herein, in particular embodiments, the hypomethylation is in the promoter sequence of the regulatory region. More particularly, the hypomethylation may be in a CpG dinucleotide motif of the promoter. In some embodiments, the regulatory region of the at least one gene comprises a BORIS binding site.

In particular embodiments, the methylation status of the regulatory regions of TKTL1, GRIN1, and GPR17 is determined. In other embodiments, the methylation status of the regulatory regions of MAGEA2, MAGEA3, MAGEA4, MAGEA11 and H19 is determined. In other embodiments, the methylation status of the regulatory regions of MAGEA3, MAGEA12, MAGEA4, MAGEA1, MAGEA5, and NY-ESO-1 is determined.

In another embodiment, there are provided methods for diagnosing a disorder in a subject having or at risk of developing a cell proliferative disorder. The method includes contacting a nucleic acid-containing sample from cells of the subject with an agent that provides a determination of the methylation state of at least one regulatory region of a gene, wherein the at least one regulatory region is hypomethylated in a cell undergoing unregulated cell growth as compared to a corresponding normal cell; and identifying hypomethylation of the regulatory region in the nucleic acid-containing sample, as compared to the same region of the at least one regulatory region in a subject not having the proliferative disorder, wherein hypomethylation is indicative of a subject having or at risk of developing the proliferative disorder.

The term “cell proliferative disorder” as used herein refers to malignant as well as non-malignant cell populations which often differ from the surrounding tissue both morphologically and genotypically. In some embodiments, the cell proliferative disorder is a cancer. In particular embodiments the cancer may be a carcinoma or a sarcoma. A cancer can include, but is not limited to, head cancer, neck cancer, head and neck cancer, lung cancer, breast cancer, prostate cancer, colorectal cancer, esophageal cancer, stomach cancer, leukemia/lymphoma, uterine cancer, skin cancer, endocrine cancer, urinary cancer, pancreatic cancer, gastrointestinal cancer, ovarian cancer, cervical cancer, and adenomas. In one aspect, the cancer is head and neck cancer. In another aspect, the cancer is lung cancer.

The nucleic acid-containing sample for use in the invention methods may be virtually any biological sample that contains nucleic acids from the subject. The biological sample can be a tissue sample which contains 1 to 10,000,000, 1000 to 10,000,000, or 1,000,000 to 10,000,000 somatic cells. However, it is possible to obtain samples that contain smaller numbers of cells, even a single cell in embodiments that utilize an amplification protocol such as PCR. The sample need not contain any intact cells, so long as it contains sufficient material (e.g., protein or genetic material, such as RNA or DNA) to assess methylation status or gene expression levels. In some embodiments the nucleic acid-containing sample is obtained from cells are from a sample selected from the group consisting of a tissue sample, a frozen tissue sample, a biopsy specimen, a surgical specimen, a cytological specimen, whole blood, bone marrow, cerebral spinal fluid, peritoneal fluid, pleural fluid, lymph fluid, serum, mucus, plasma, urine, chyle, stool, ejaculate, sputum, nipple aspirate and saliva.

A biological or tissue sample can be drawn from any tissue that is susceptible to cancer. For example, the tissue may be obtained by surgery, biopsy, swab, stool, or other collection method. The biological sample for methods of the present invention can be, for example, a sample from colorectal tissue, or in certain embodiments, can be a blood sample, or a fraction of a blood sample such as a peripheral blood lymphocyte (PBL) fraction. Methods for isolating PBLs from whole blood are well known in the art. An example of such a method is provided in the Example section herein. In addition, it is possible to use a blood sample and enrich the small amount of circulating cells from a tissue of interest, e.g., lung, colon, breast, etc. using a method known in the art.

In the present invention, the subject is typically a human, but also can be any mammal, including, but not limited to, a dog, cat, rabbit, cow, rat, horse, pig, or monkey.

Numerous methods for analyzing methylation status of a gene or regulatory region are known in the art and can be used in the methods of the present invention to identify hypomethylation. As illustrated in the Examples herein, analysis of methylation can be performed by bisulfite genomic sequencing.

Bisulfite ions, for example, sodium bisulfite, convert non-methylated cytosine residues to bisulfite modified cytosine residues. The bisulfite ion treated gene sequence can be exposed to alkaline conditions, which convert bisulfite modified cytosine residues to uracil residues. Sodium bisulfite reacts readily with the 5,6-double bond of cytosine (but poorly with methylated cytosine) to form a sulfonated cytosine reaction intermediate that is susceptible to deamination, giving rise to a sulfonated uracil. The sulfonate group can be removed by exposure to alkaline conditions, resulting in the formation of uracil. The DNA can be amplified, for example, by PCR, and sequenced to determine whether CpG sites are methylated in the DNA of the sample. Uracil is recognized as a thymine by Taq polymerase and, upon PCR, the resultant product contains cytosine only at the position where 5-methylcytosine was present in the starting template DNA. One can compare the amount or distribution of uracil residues in the bisulfite ion treated gene sequence of the test cell with a similarly treated corresponding non-methylated gene sequence. A decrease in the amount or distribution of uracil residues in the gene from the test cell indicates methylation of cytosine residues in CpG dinucleotides in the gene of the test cell. The amount or distribution of uracil residues also can be detected by contacting the bisulfite ion treated target gene sequence, following exposure to alkaline conditions, with an oligonucleotide that selectively hybridizes to a nucleotide sequence of the target gene that either contains uracil residues or that lacks uracil residues, but not both, and detecting selective hybridization (or the absence thereof) of the oligonucleotide.

In another embodiment, the gene is contacted with hydrazine, which modifies cytosine residues, but not methylated cytosine residues, then the hydrazine treated gene sequence is contacted with a reagent such as piperidine, which cleaves the nucleic acid molecule at hydrazine modified cytosine residues, thereby generating a product comprising fragments. By separating the fragments according to molecular weight, using, for example, an electrophoretic, chromatographic, or mass spectrographic method, and comparing the separation pattern with that of a similarly treated corresponding non-methylated gene sequence, gaps are apparent at positions in the test gene contained methylated cytosine residues. As such, the presence of gaps is indicative of methylation of a cytosine residue in the CpG dinucleotide in the target gene of the test cell.

Modified products can be detected directly, or after a further reaction which creates products which are easily distinguishable. Means which detect altered size and/or charge can be used to detect modified products, including but not limited to electrophoresis, chromatography, and mass spectrometry. Examples of such chemical reagents for selective modification include hydrazine and bisulfite ions. Hydrazine-modified DNA can be treated with piperidine to cleave it. Bisulfite ion-treated DNA can be treated with alkali. Other means which are reliant on specific sequences can be used, including but not limited to hybridization, amplification, sequencing, and ligase chain reaction, Combinations of such techniques can be uses as is desired.

In another example, methylation status may be assessed using real-time methylation specific PCR. For example, the methylation level of the promoter region of one or more of the target genes can be determined by determining the amplification level of the promoter region of the target gene based on amplification-mediated displacement of one or more probes whose binding sites are located within the amplicon. In general, real-time quantitative methylation specific PCR is based on the continuous monitoring of a progressive fluorogenic PCR by an optical system. Such PCR systems are well-known in the art and usually use two amplification primers and an additional amplicon-specific, fluorogenic hybridization probe that specifically binds to a site within the amplicon. The probe can include one or more fluorescence label moieties. For example, the probe can be labeled with two fluorescent dyes: 1) a 6-carboxy-fluorescein (FAM), located at the 5′-end, which serves as reporter, and 2) a 6-carboxy-tetramethyl-rhodamine (TAMRA), located at the 3′-end, which serves as a quencher. When amplification occurs, the 5′-3′ exonuclease activity of the Taq DNA polymerase cleaves the reporter from the probe during the extension phase, thus releasing it from the quencher. The resulting increase in fluorescence emission of the reporter dye is monitored during the PCR process and represents the number of DNA fragments generated.

In other embodiments, hypomethylation can be identified through nucleic acid sequencing after bisulfite treatment to determine whether a uracil or a cytosine is present at specific location within a gene or regulatory region. If uracil is present after bisulfite treatment, then the nucleotide was unmethylated. Hypomethylation is present when there is a measurable decrease in methylation.

In an alternative embodiment, the method for analyzing methylation of the target gene can include amplification using a primer pair specific for methylated residues within a the target gene. In these embodiments, selective hybridization or binding of at least one of the primers is dependent on the methylation state of the target DNA sequence (Herman et al., Proc. Natl. Acad. Sci. USA, 93:9821 (1996)). For example, the amplification reaction can be preceded by bisulfite treatment, and the primers can selectively hybridize to target sequences in a manner that is dependent on bisulfite treatment. For example, one primer can selectively bind to a target sequence only when one or more base of the target sequence is altered by bisulfite treatment, thereby being specific for a methylated target sequence.

Other methods are known in the art for determining methylation status of a target gene, including, but not limited to, array-based methylation analysis (see Gitan et al., Genome Res 12:158-64, 2002) and Southern blot analysis.

Methods using an amplification reaction can utilize a real-time detection amplification procedure. For example, the method can utilize molecular beacon technology (Tyagi S., et al., Nature Biotechnology, 14: 303 (1996)) or Taqman™ technology (Holland, P. M., et al., Proc. Natl. Acad. Sci. USA, 88:7276 (1991)).

In addition, methyl light (Trinh B N, Long T I, Laird P W. DNA methylation analysis by MethyLight technology, Methods, 25(4):456-62 (2001), incorporated herein in its entirety by reference), Methyl Heavy (Epigenomics, Berlin, Germany), or SNuPE (single nucleotide primer extension) (See e.g., Watson D., et al., Genet Res. 75(3):269-74 (2000)) can be used in the methods of the present invention related to identifying altered methylation of the genes or regulatory regions provided herein. Additionally, methyl light, methyl heavy, and array-based methylation analysis can be performed, by using bisulfite treated DNA that is then PCR-amplified, against microarrays of oligonucleotide target sequences with the various forms corresponding to unmethylated and methylated DNA.

The degree of methylation in the DNA associated with the gene or genes or regulatory regions thereof, may be measured by fluorescent in situ hybridization (FISH) by means of probes which identify and differentiate between genomic DNAs, which exhibit different degrees of DNA methylation. FISH is described in the Human chromosomes: principles and techniques (Editors, Ram S. Verma, Arvind Babu Verma, Ram S.) 2nd ed., New York: McGraw-Hill, 1995, and de Capoa A., Di Leandro M., Grappelli C., Menendez F., Poggesi I., Giancotti P., Marotta, M. R., Spano A., Rocchi M., Archidiacono N., Niveleau A. Computer-assisted analysis of methylation status of individual interphase nuclei in human cultured cells. Cytometry. 31:85-92, 1998 which is incorporated herein by reference. In this case, the biological sample will typically be any which contains sufficient whole cells or nuclei to perform short term culture. Usually, the sample will be a tissue sample that contains 10 to 10,000, or, for example, 100 to 10,000, whole somatic cells.

In other embodiments, methylation-sensitive restriction endonucleases can be used to detect methylated CpG dinucleotide motifs. Such endonucleases may either preferentially cleave methylated recognition sites relative to non-methylated recognition sites or preferentially cleave non-methylated relative to methylated recognition sites. Examples of the former are Acc III, Ban I, BstN I, Msp I, and Xma I. Examples of the latter are Ace II, Ava I, BssH II, BstU I, Hpa II, and Not I. Alternatively, chemical reagents can be used which selectively modify either the methylated or non-methylated form of CpG dinucleotide motifs.

In some embodiments, hypomethylation of the target gene is detected by detecting increased expression of the that gene. Expression of a gene can be assessed using any means known in the art. Typically expression is assessed and compared in test samples and control samples which may be normal, non-malignant cells. The test samples may contain cancer cells or pre-cancer cells or nucleic acids from them. Methods employing hybridization to nucleic acid probes can be employed for measuring specific mRNAs. Such methods include using nucleic acid probe arrays (microarray technology), in situ hybridization, and using Northern blots. Messenger RNA can also be assessed using amplification techniques, such as RT-PCR. Advances in genomic technologies now permit the simultaneous analysis of thousands of genes, although many are based on the same concept of specific probe-target hybridization. Sequencing-based methods are an alternative; these methods started with the use of expressed sequence tags (ESTs), and now include methods based on short tags, such as serial analysis of gene expression (SAGE) and massively parallel signature sequencing (MPSS). Differential display techniques provide yet another means of analyzing gene expression; this family of techniques is based on random amplification of cDNA fragments generated by restriction digestion, and bands that differ between two tissues identify cDNAs of interest. Moreover, specific proteins can be assessed using any convenient method including immunoassays and immuno-cytochemistry but are not limited to that. Most such methods will employ antibodies which are specific for the particular protein or protein fragments. The sequences of the mRNA (cDNA) and proteins of the target genes of the present invention are known in the art and publicly available.

As used herein, the term “selective hybridization” or “selectively hybridize” refers to hybridization under moderately stringent or highly stringent physiological conditions, which can distinguish related nucleotide sequences from unrelated nucleotide sequences.

As known in the art, in nucleic acid hybridization reactions, the conditions used to achieve a particular level of stringency will vary, depending on the nature of the nucleic acids being hybridized. For example, the length, degree of complementarity, nucleotide sequence composition (for example, relative GC:AT content), and nucleic acid type, i.e., whether the oligonucleotide or the target nucleic acid sequence is DNA or RNA, can be considered in selecting hybridization conditions. An additional consideration is whether one of the nucleic acids is immobilized, for example, on a filter. Methods for selecting appropriate stringency conditions can be determined empirically or estimated using various formulas, and are well known in the art (see, for example, Sambrook et al., supra, 1989).

An example of progressively higher stringency conditions is as follows: 2×SSC/0.1% SDS at about room temperature (hybridization conditions); 0.2×SSC/0.1% SDS at about room temperature (low stringency conditions); 0.2×SSC/0.1% SDS at about 42° C. (moderate stringency conditions); and 0.1×SSC at about 68° C. (high stringency conditions). Washing can be carried out using only one of these conditions, for example, high stringency conditions, or each of the conditions can be used, for example, for 10 to 15 minutes each, in the order listed above, repeating any or all of the steps listed.

The term “nucleic acid molecule” is used broadly herein to mean a sequence of deoxyribonucleotides or ribonucleotides that are linked together by a phosphodiester bond. As such, the term “nucleic acid molecule” is meant to include DNA and RNA, which can be single stranded or double stranded, as well as DNA/RNA hybrids. Furthermore, the term “nucleic acid molecule” as used herein includes naturally occurring nucleic acid molecules, which can be isolated from a cell, for example, a particular gene of interest, as well as synthetic molecules, which can be prepared, for example, by methods of chemical synthesis or by enzymatic methods such as by the polymerase chain reaction (PCR), and, in various embodiments, can contain nucleotide analogs or a backbone bond other than a phosphodiester bond.

The terms “polynucleotide” and “oligonucleotide” also are used herein to refer to nucleic acid molecules. Although no specific distinction from each other or from “nucleic acid molecule” is intended by the use of these terms, the term “polynucleotide” is used generally in reference to a nucleic acid molecule that encodes a polypeptide, or a peptide portion thereof, whereas the term “oligonucleotide” is used generally in reference to a nucleotide sequence useful as a probe, a PCR primer, an antisense molecule, or the like. Of course, it will be recognized that an “oligonucleotide” also can encode a peptide. As such, the different terms are used primarily for convenience of discussion.

A polynucleotide or oligonucleotide comprising naturally occurring nucleotides and phosphodiester bonds can be chemically synthesized or can be produced using recombinant DNA methods, using an appropriate polynucleotide as a template. In comparison, a polynucleotide comprising nucleotide analogs or covalent bonds other than phosphodiester bonds generally will be chemically synthesized, although an enzyme such as T7 polymerase can incorporate certain types of nucleotide analogs into a polynucleotide and, therefore, can be used to produce such a polynucleotide recombinantly from an appropriate template.

In yet another embodiment of the invention, there are provided methods of determining the prognosis of a subject having a cell proliferative disorder. The method includes determining the methylation state of at least one regulatory region of a gene in a nucleic acid sample from the subject, wherein hypomethylation as compared to a corresponding normal cell in the subject or a subject not having the disorder, is indicative of a poor prognosis.

In still another embodiment of the present invention, there are provided methods of ameliorating a cell proliferative disorder in a subject in need thereof. The method includes administering to the subject an agent that methylates at least one regulatory region in a gene that is demethylated as compared to a subject not having the disorder, thereby reducing expression of the at least one gene and ameliorating the cell proliferative disorder.

Methylating agents are known in the art and include, for example, alkylating agents such as nitrosureas, triazenes, and imidazotetrzines. In particular embodiments, the methylating agent is delivered locally to a tumor site or systemically by targeted drug delivery.

Agents that methylate the demethylated gene can be contacted with cells in vitro or in vivo for the purpose of restoring normal gene expression to the cell. Efficacy of the treatment can be assessed by detecting increased expression or methylation of a gene selected from the group consisting of TKTL1, H19, MAGEA2, MAGEA3, MAGEA4, MAGEA11, GPR17, GRIN1, C19ORF28, MAGEA12, MAGEA1, MAGEA5, NY-ESO-1, MAGEA9, MAGEA6, MAGEB2, CT45-2, SBSN, G6PD, ZNF711, CrispL, KRT86, KIPV467, KRT81, CSPG5, PP1R14A, KISS1R, KIAA1937 protein, SOX30, DEAD, and KBGP.

In a further embodiment of the present invention, there are provided methods of identifying a gene activated by hypomethylation. The method includes comparing an expression analysis of a cell treated with an agent that reduces methylation to an expression analysis of a control cell not treated with the agent, wherein an increase in expression of a gene is indicative of a gene activated by demethylation. In one aspect, the cell is from a minimally transformed cell line. In some embodiments, the method may further include an expression analysis of a tissue sample and a tumor sample from the same tissue of origin as the treated cell, wherein an increase in expression of a gene in a tumor sample as compared to a normal sample is correlated to the genes activated by demethylation in the treated cell. The method may also include sequence analysis to identify CpG dinucleotide motifs in the regulatory region, or particularly the promoter of identified genes. Determination of the methylation status of the identified genes in tumor and corresponding normal tissue samples may also be included.

The demethylating agent can be a methyltransferase inhibitor such as 5-aza-2′-deoxycytidine (DAC). In one aspect, the histone deacetylase inhibitor trichostatin (TSA) is used to treat cells for further determination of the methylation status. In yet another aspect, a combination of 5 aza T deoxycytidine (5Aza-dC) and trichostatin (TSA) is utilized.

The data provided herein suggest that multiple solid tumor types undergo activation of candidate proto-oncogenes with associated demethylation in a coordinated fashion in individual tumors. Transformation-associated effects of BORIS expressed ectopically in BORIS-negative cell lines as well as growth effects with individual target genes that have been shown to be epigenetically activated and expressed by BORIS are demonstrated herein. However, this does not rule out the contribution of as yet unidentified genes to BORIS related effects or a cooperative effect between identified target genes. Cancer testes antigens including four of the genes identified herein, MAGE A2, A3, A4, A11, are part of the melanoma antigen family A (MAGE-A) family of genes initially discovered as targets for immunotherapy due to their near exclusive tumor-specific expression, but the MAGE-A family plays a functional role in cancer development. MAGEA2 binds to p53-responsive promoters and leads to assembly of a p53/MAGEA2/HDAC3 protein complex, resulting in transcriptional silencing of genes ordinarily activated by p53 because of histone deacetylation. Additionally, different MAGE-A family members can repress downstream targets of p53, and studies have also linked MAGE-A family overexpression to chemo-resistance, and MAGE family members have been shown to increase cell growth and inactivate TSG activity. Recently, MAGEA has been shown to repress p53-dependent apoptosis, and has been associated with resistance to taxanes and alkylating agents in gastric cancer.

As provided in Example 1, expression of the MAGE-A family and expression of H19 appeared to be significantly related in primary tumors, supported by data indicating that these targets are controlled by common methylation-specific transcription factors. H19 forms half of the best-studied example of imprinted-gene regulation, the IGF2/H19 locus. IGF2 (insulin-like growth factor 2) is expressed uniquely from the parental allele achieved by monoallelic methylation of the imprinting control region (ICR) at 11p15.5. Aberrant hypomethylation at this locus is one cause of Silver-Russell syndrome—a disease of asymmetry or hemihypertropy associated with increased risk of malignancies including craniopharyngioma, testicular seminoma, hepatocellular carcinoma, and Wilms tumor. Additionally, several cases of familial Beckwith-Wiedemann syndrome (BWS), with and without Wilms' tumors, have been shown to be caused by microdeletions of the methylation-specific CTCF binding sites in the H19 ICR, a rare familial cancer syndrome linked to epigenetics.

Other identified proto-oncogenes provided herein have been implicated recently in tumorigenesis. TKTL1 protein expression is correlated to worse patient outcome in patients with invasive colon and urothelial tumors, and investigators hypothesize that enhanced TKTL1 expression in tumors increases oxygen-independent glucose usage (Krockenberger et al., Int J Gynecol Cancer 17:101-6, 2007). In addition, over-expression of TKTL1 has since been validated as a potential biomarker and treatment target in breast cancer (Foldi et al, Oncol Rep 17:841-5, 2007). GPR17 and GR1M have not been implicated in carcinogenesis to date. Although growth promoting effects of C19ORF28 have not been demonstrated, this does not exclude the possibility that overexpression of this and any of our other targets may contribute to a malignant phenotype via other mechanisms—e.g., motility, invasion, angiogenesis, or apoptosis resistance—or that it may cooperate with other identified targets to produce phenotypic effects.

The epigenetic reactivation of TKTL1, H19, MAGEA2, MAGEA3, MAGEA4, MAGEA11, GPR17, GRIN1, and C19ORF28, genes located at diverse chromosomal loci, appears to occur simultaneously in individual primary tumors from multiple tumor types. This concurrent genome-wide, promoter-specific hypomethylation that results in derepression of many potential oncogenes raises the possibility of a demethylator phenotype analogous to the CpG island methylator phenotype (GIMP) initially noted in colon cancer. Many proto-oncogenes are members of the cancer testes antigen family which are ordinarily repressed via epigenetic mechanisms during development. While not wishing to be bound by any particular theory, an attractive hypothesis is that this phenomenon represents the coordinated, but pathologic reversal of developmental epigenetic regulatory patterns in cancer cells. The validity of whole-genome integrative approach to screening for epigenetically-activated genes associated with malignancy provided herein is confirmed by the appearance of H19 and the MAGE-A family members which have been reported to be controlled by epigenetic activation and show silencing in normal cells. Two separate groups among the nine genes identified in Example 1 showed statistically significant correlations for patterns of expression: 1) MAGEA family members with H19 and 2) TKTL1, GPR17, and GRIN. These groups were also identified according to promoter homology, implicating the participation of promoter-specific binding activity in the coordinated expression of each of these groups and suggesting the existence of additional common transcriptional activators that recognize the specific demethylated promoter sequences of these genes. The strict correlation of BORIS expression with aberrant expression of multiple growth-promoting proto-oncogenes in a variety of solid tumors reinforces the postulated role for BORIS as a key participant in aberrant demethylation and transcriptional activation of putative oncogenes. This concept is supported by cell line experiments demonstrating that BORIS expression by itself is sufficient to simultaneously demethylate and activate the transcription of these genes. It is of great interest to define the factors with which BORIS cooperates to induce these epigenetic and expression changes. Recently, a role for BORIS in histone demethylation and chromatin remodeling has been demonstrated. Moreover, regardless of mechanism, the data provide strong evidence for consideration of BORIS as a dominant controlling factor for facilitating epigenetic alterations associated with coordinated demethylation and reactivation of target genes that are of high value as potential therapeutic and diagnostic targets for NSCLC, HNSCC, and other tumors.

This simultaneous reactivation of multiple targets provides a significant challenge to the understanding of the collective, and perhaps cooperative, effects of this phenomenon in cell transformation. In particular, single targets may depend on concurrent activation of, and interaction with, other family members for oncogenic effect. Other investigators have found some evidence of coordination of cancer testes antigen family expression and the possibility of direct interactions. In addition, only the top 26/106 possible targets identified after integrative analysis in a single solid tumor type were selected for further analysis. Future studies of the remaining genes, as well as use of normal cell lines and tumors derived from other tissues in an integrative approach, would be expected to allow for discovery of additional, novel, epigenetically-controlled genes that may also act collaboratively to induce malignant transformation.

Due to lack of primary tumor data on a larger array platform a nonintegrative approach was also used, which resulted in ultimate validation of 4.4% of the targets (2/45) compared to the integrative results that produced a 27% hit rate (7/26), reflecting a higher ability to validate targets in primary tumor when these data are included in initial discovery strategies. Additional analysis of other targets that are significantly differentially regulated may also yield additional epigenetically derepressed targets. Finally, these data have therapeutic implications for demethylation therapy and targeting of therapy. The active investigation of pharmacologic demethylating agents as therapy for malignancy based on reversal of silencing of tumor suppressor genes may have unintended effects. It is possible that in certain tissues this may result in reactivation of developmentally repressed proto-oncogene targets, with the unintended effect of promoting late, second primary tumors. However, modulation of a pathway that involves the coordinated derepression of a series of growth-promoting protooncogene candidates and a key transcriptional effector, BORIS, may provide a significant opportunity for directed therapeutic intervention that simultaneously targets multiple oncogenic pathways.

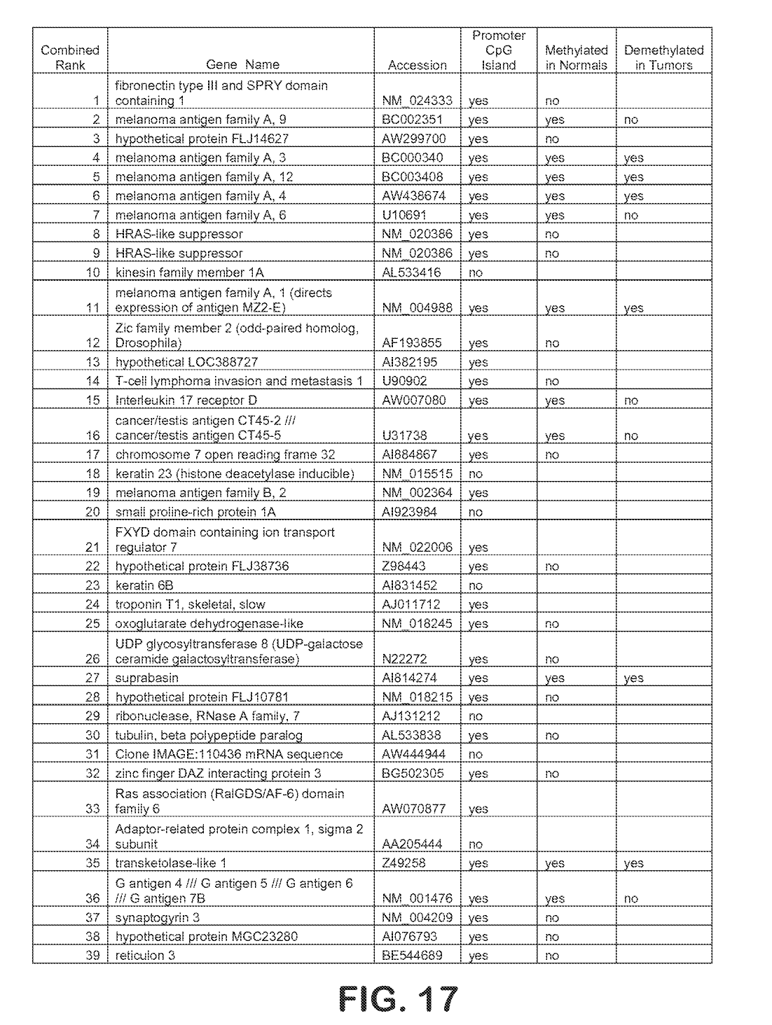

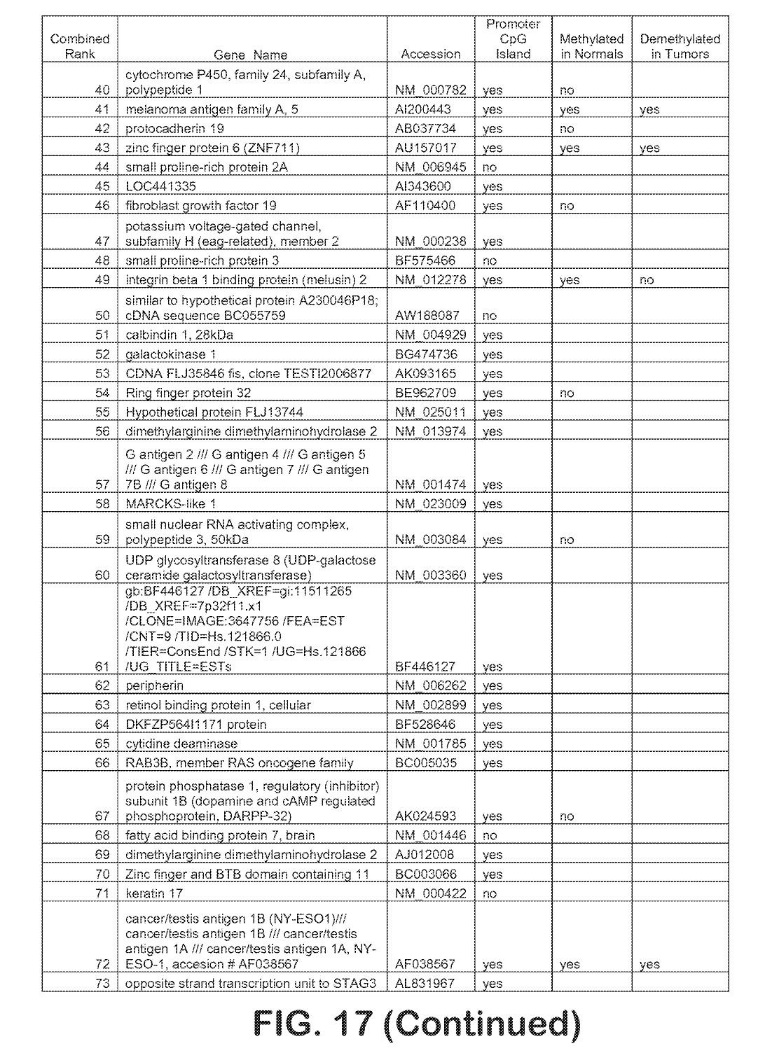

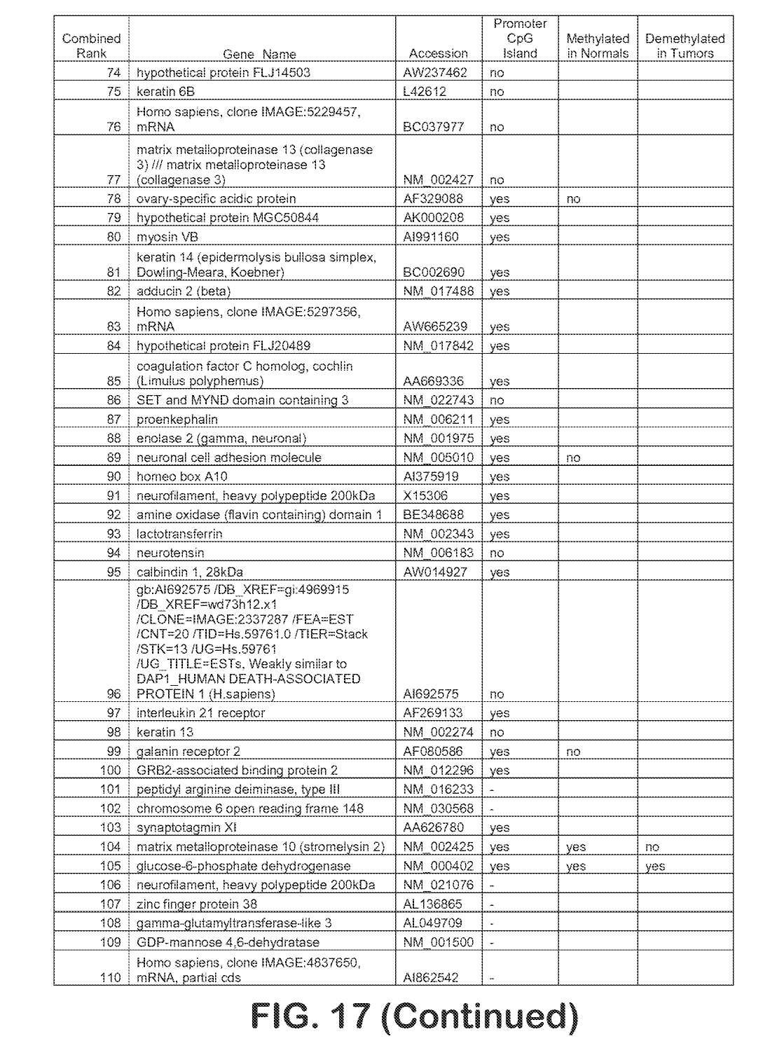

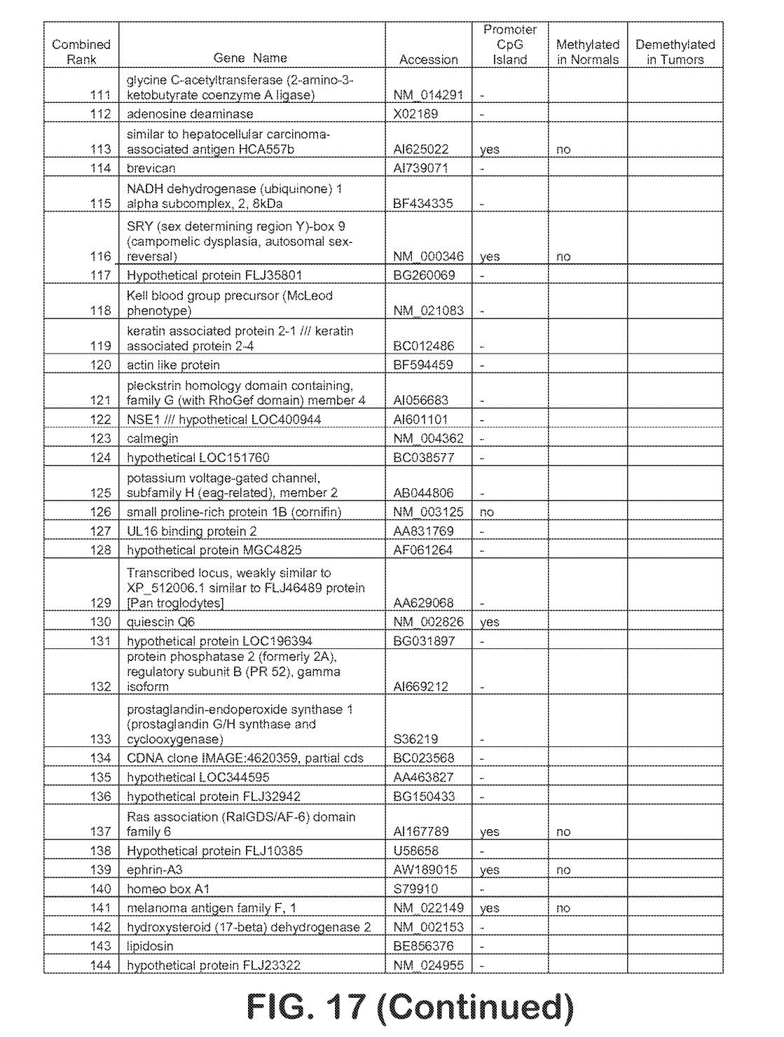

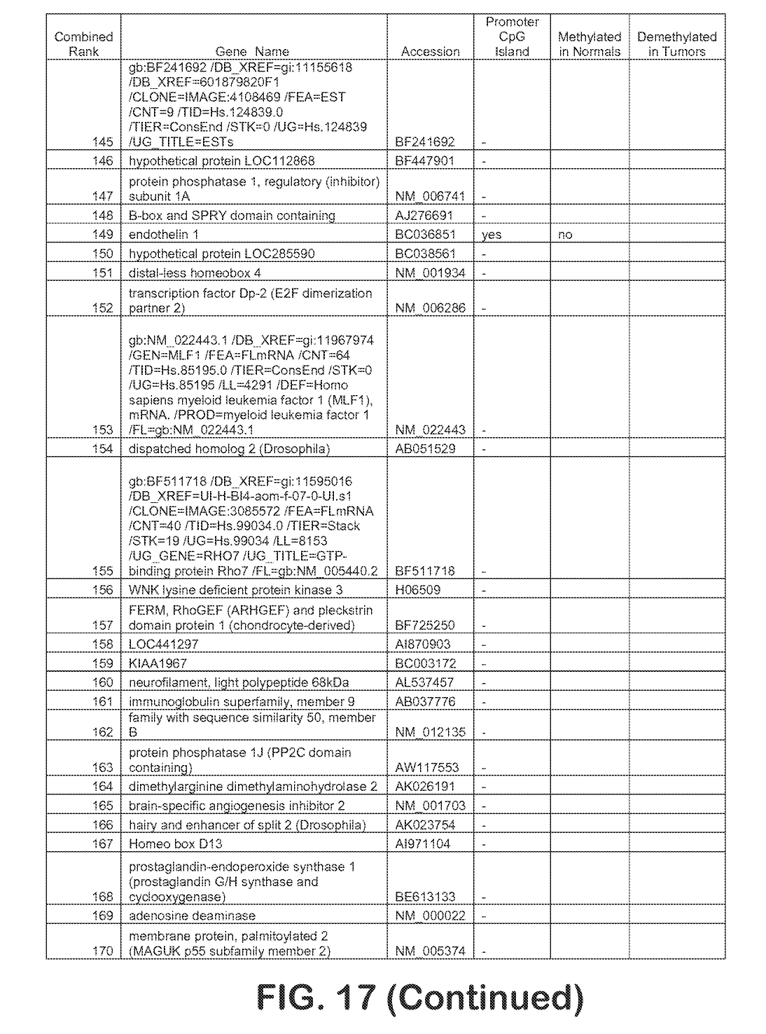

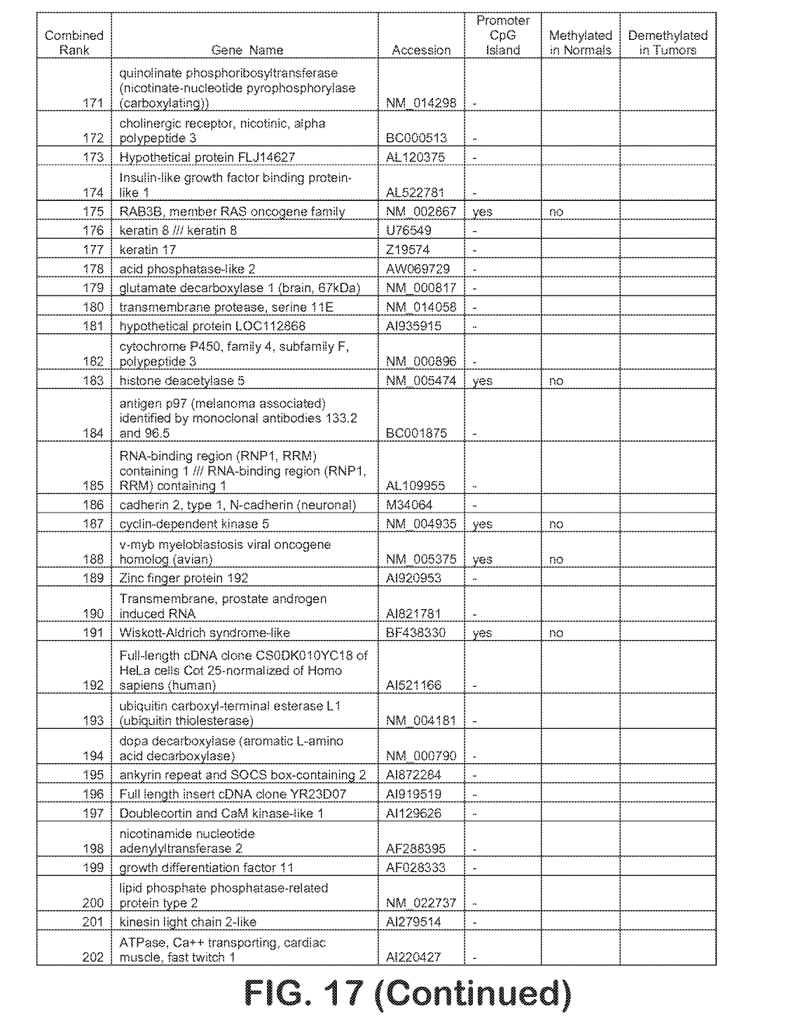

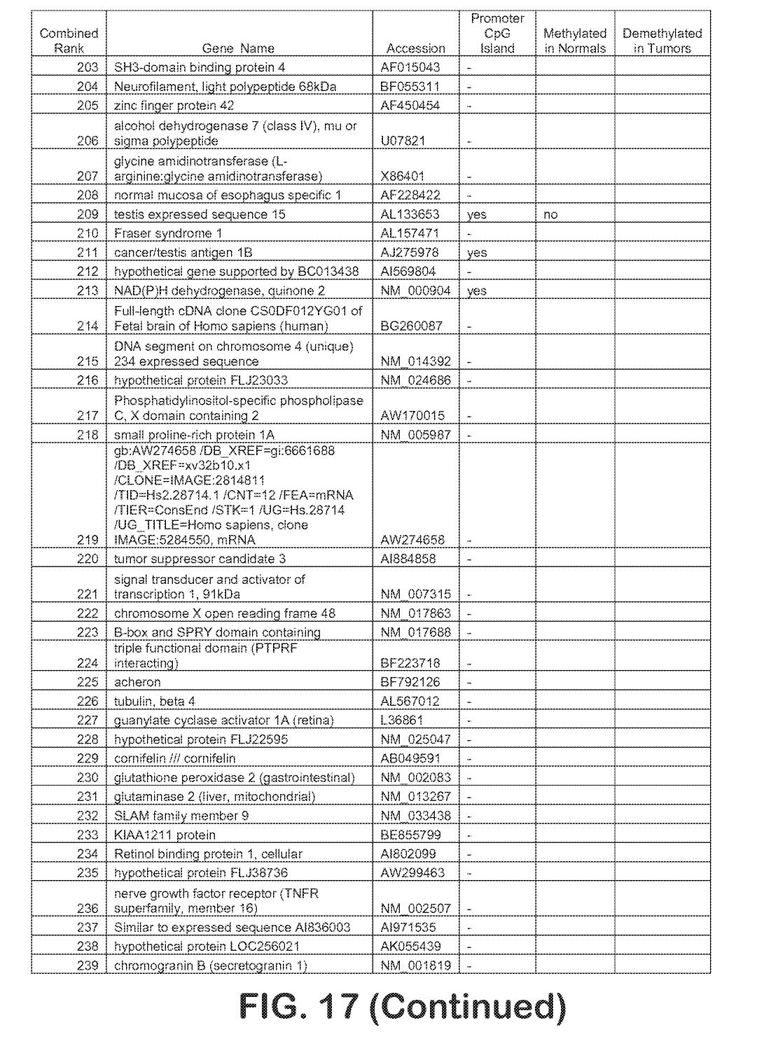

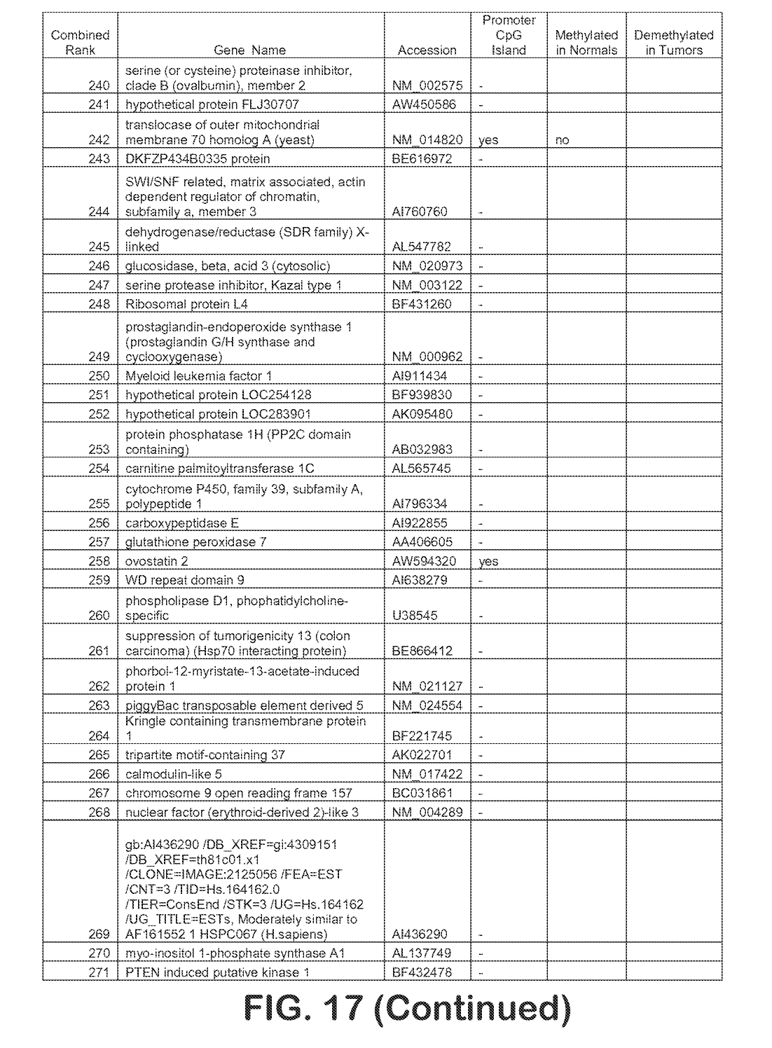

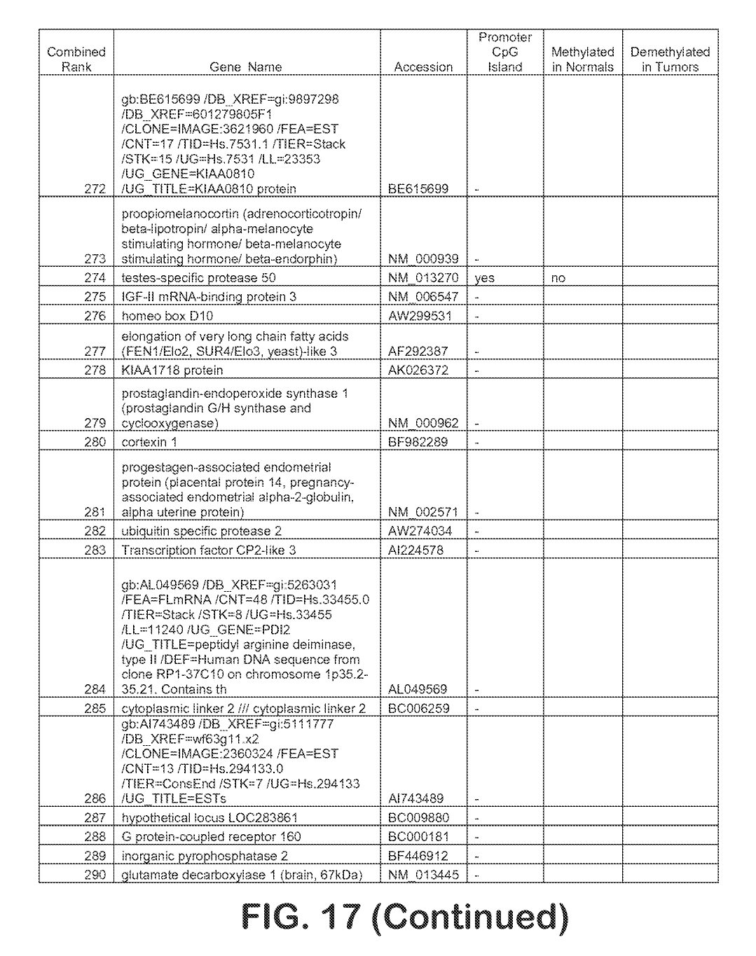

In the study provided in Example 2, an integrative epigenetic screening approach was used to specifically identify coordinately expressed genes in human NSCLC whose transcription is driven by promoter demethylation. From the over 47,000 transcripts incorporated in the Affymetrix Human Genome U133 Plus 2.0 expression platform, 10 genes were identified that showed both differential overexpression and promoter region hypomethylation in NSCLC. Surprisingly, 6 of the 10 genes discovered via this approach were known CT-X antigens, MAGEA3, MAGEA12, MAGEA4, MAGEA1, MAGEA5 and NY-ESO-1. Four additional CTAs, MAGEA9, MAGEA6, MAGEB2 and CT45-2, were within the top 20 on the rank list provided herein; however, these genes did not meet the screening selection criteria due to failure to show complete methylation of promoter regions in a separate cohort of normal lung tissue by bisulfite sequencing (FIG. 17). It is possible that, by using less stringent selection criteria, these and other genes would be identified as differentially expressed, albeit with incomplete promoter methylation patterns in normal lung tissue. In this study, only the top 55 of the 290 possible targets identified after integrative analysis in a single solid tumor type were selected for further analysis. It is expected that further investigation of the remaining genes, as well as the use of normal cell lines and tumors derived from additional tissue types in an integrative approach, will allow for discovery of additional, novel, epigenetically-controlled genes that may also show coordinated expression in tumors and serve as possible targets for screening and immunogenic therapy.

Although some of the CTAs identified using the integrative technique provided have previously been shown to be expressed to some degree in NSCLC; the demonstration of a high degree of coordinated expression in a large sample set related to epigenetic unmasking is a new finding. In a previous study of 19 lung carcinoma cell lines expressing various MAGEA family members, there was nearly complete concordance between the RT-PCR and IHC results. Thus the use of quantitative RT-PCR is a valid method for detecting CT antigen expression, especially when dealing with primary tissue where it is usually not possible to isolate sufficient quantities of protein for analysis. In addition, this same study showed that 44% of the 187 NSCLC samples tested on tissue microarrays stained positive, to some degree, for MAGEA family expression, supporting the fact that CTAs are expressed at the protein level in NSCLC.

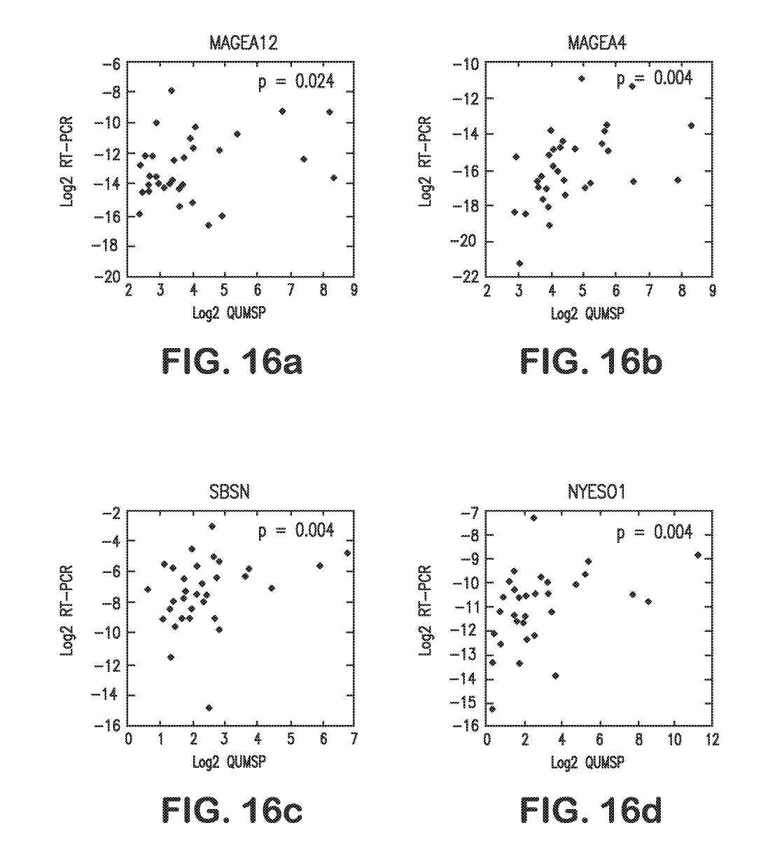

Four target genes showed a significant positive correlation between mRNA expression and promoter hypomethylation, MAGEA12, MAGEA4, SBSN and NY-ESO-1 (FIG. 16). TKTL1, MAGEA5 and MAGEA3 also showed a positive correlation between demethylation and expression, but missed significance. The lack of correlation between expression and hypomethylation in some of the target genes is expected given the fact that multiple other mechanisms such as point mutations, insertions, deletions and loss of heterozygosity could be involved in gene expression regulation in NSCLC. Alternatively, a larger sample size may facilitate the ability to define a closer association between promoter methylation status and expression in these genes.

In addition to the 6 mentioned CT-X antigens, elucidated 4 additional target genes were elucidated that were showed to be coordinately expressed with the known CTAs and demethylated in tumors. Interestingly, three of these 4 genes are encoded by the X chromosome, TKTL-1, ZNF-711 and G6PD. TKTL1 has been correlated with worse outcomes in patients with invasive colon and urothelial tumors, oxygen-independent glucose usage and validated as a potential biomarker in breast cancer. SBSN, ZNF-711 and G6PD have not previously been associated with tumor specific expression or carcinogenesis.

CTAs are attractive targets for tumor immunotherapy because of their restricted expression patterns in normal human tissue. Currently, demethylating agents and HDAC inhibitors are being studied as adjuvant treatment options for NSCLC and other human malignancies, and combinations of these drugs continue to undergo bench-top and clinical investigation. These epigenetic therapies are being utilized based on data that suggests that methylation of tumor suppressor genes plays a fundamental role in tumor formation, progression, and recurrence after resection. Promoter region methylation of certain genes in resected NSCLC specimens was recently shown to be associated with recurrence of the tumor and poorer patient outcomes. An additional study has previously shown that NY-ESO-1 and MAGEA3 are upregulated in a proportion of patients treated with 5-aza-2′-deoxycytidine in cancers involving the lung, esophagus, or pleura. With the data herein suggesting that multiple CTAs are coordinately expressed in NSCLC and demethylation coordinately upregulates multiple known CTAs and associated genes from the target list, combining the use of demethylating agents with immunotherapy targeted against these genes that might be derepressed after treatment with 5-AZA and other demethylating agents may be useful. Targeting multiple CTAs that are coordinately expressed would help to improve the efficacy seen with monovalent immunologic agents.

The function of these genes expressed uniquely in NSCLC has not been well explored. There are data that indicate that MAGEA family members have growth promoting effects, and CTA members have been associated with biologic pathways that support a malignant phenotype. Additional analyses of the genes that are aberrantly expressed via promoter demethylation in NSCLC would be expected to demonstrate functional effects that contribute to carcinogenesis.

Using an integrative analysis combining pharmacologic demethylation and previously published primary tissue array data, a common epigenetic mechanism for the coordinated expression of CTAs and additional targets that may serve as targets for immunotherapy have been defined. Accordingly, in a further embodiment of the present invention, the integrative epigenetic analysis provided herein may be used to identify antigens expressed in cancer cells, in particular groups of antigens that are coordinately expressed in cancer. Such antigens may be used as targets for anti-cancer immunotherapy. Further, antigens identified as having a common epigenetic mechanism for coordinated expression may be targeted in combination in immunotherapy, including antigens containing CpG islands.

In another embodiment of the present invention, there are provided methods for determining whether a subject is responsive to a particular therapeutic regimen. The method includes determining the methylation status of one or more genes or regulatory regions thereof, selected from the group consisting of TKTL1, H19, MAGEA2, MAGEA3, MAGEA4, MAGEA11, GPR17, GRIN1, C19ORF28, MAGEA12, MAGEA1, MAGEA5, NY-ESO-1, MAGEA9, MAGEA6, MAGEB2, CT45-2, SBSN, G6PD, and ZNF711, wherein hypomethylation of the gene or regulatory region thereof as compared with a normal subject is indicative of a subject who is responsive to the therapeutic regimen.

In certain embodiments, therapeutic regimen is administration of a chemotherapeutic agent. While not wanting to be limiting, chemotherapeutic agents include antimetabolites, such as methotrexate, DNA cross-linking agents, such as cisplatin/carboplatin; alkylating agents, such as canbusil; topoisomerase I inhibitors such as dactinomycin; microtubule inhibitors such as taxol (paclitaxol), and the like. Other chemotherapeutic agents include, for example, a vinca alkaloid, mitomycin-type antibiotic, bleomycin-type antibiotic, antifolate, colchicine, demecolcine, etoposide, taxane, anthracycline antibiotic, doxorubicin, daunorubicin, caminomycin, epirubicin, idarubicin, mitoxanthrone, 4-dimethoxy-daunomycin, 11-deoxydaunorubicin, 13-deoxydaunorubicin, adriamycin-14-benzoate, adriamycin-14-octanoate, adriamycin-14-naphthaleneacetate, amsacrine, carmustine, cyclophosphamide, cytarabine, etoposide, lovastatin, melphalan, topetecan, oxalaplatin, chlorambucil, methotrexate, lomustine, thioguanine, asparaginase, vinblastine, vindesine, tamoxifen, or mechlorethamine. While not wanting to be limiting, therapeutic antibodies include antibodies directed against the HER2 protein, such as trastuzumab; antibodies directed against growth factors or growth factor receptors, such as bevacizumab, which targets vascular endothelial growth factor, and OSI-774, which targets epidermal growth factor; antibodies targeting integrin receptors, such as Vitaxin (also known as MEDI-522), and the like. Classes of anticancer agents suitable for use in compositions and methods of the present invention include, but are not limited to: 1) alkaloids, including, microtubule inhibitors (e.g., Vincristine, Vinblastine, and Vindesine, etc.), microtubule stabilizers (e.g., Paclitaxel [Taxol], and Docetaxel, Taxotere, etc.), and chromatin function inhibitors, including, topoisomerase inhibitors, such as, epipodophyllotoxins (e.g., Etoposide [VP-16], and Teniposide [VM-26], etc.), and agents that target topoisomerase I (e.g., Camptothecin and Isirinotecan [CPT-11], etc.); 2) covalent DNA-binding agents [alkylating agents], including, nitrogen mustards (e.g., Mechlorethamine, Chlorambucil, Cyclophosphamide, Ifosphamide, and Busulfan [Myleran], etc.), nitrosoureas (e.g., Carmustine, Lomustine, and Semustine, etc.), and other alkylating agents (e.g., Dacarbazine, Hydroxymethylmelamine, Thiotepa, and Mitocycin, etc.); 3) noncovalent DNA-binding agents [antitumor antibiotics], including, nucleic acid inhibitors (e.g., Dactinomycin [Actinomycin D], etc.), anthracyclines (e.g., Daunorubicin [Daunomycin, and Cerubidine], Doxorubicin [Adriamycin], and Idarubicin [Idamycin], etc.), anthracenediones (e.g., anthracycline analogues, such as, [Mitoxantrone], etc.), bleomycins (Blenoxane), etc., and plicamycin (Mithramycin), etc.; 4) antimetabolites, including, antifolates (e.g., Methotrexate, Folex, and Mexate, etc.), purine antimetabolites (e.g., 6-Mercaptopurine [6-MP, Purinethol], 6-Thioguanine [6-TG], Azathioprine, Acyclovir, Ganciclovir, Chlorodeoxyadenosine, 2-Chlorodeoxyadenosine [CdA], and 21-Deoxycoformycin [Pentostatin], etc.), pyrimidine antagonists (e.g., fluoropyrimidines [e.g., 5-fluorouracil (Adrucil), 5-fluorodeoxyuridine (FdUrd) (Floxuridine)] etc.), and cytosine arabinosides (e.g., Cytosar [ara-C] and Fludarabine, etc.); 5) enzymes, including, L-asparaginase; 6) hormones, including, glucocorticoids, such as, antiestrogens (e.g., Tamoxifen, etc.), nonsteroidal antiandrogens (e.g., Flutamide, etc.), and aromatase inhibitors (e.g., anastrozole [Arimidex], etc.); 7) platinum compounds (e.g., Cisplatin and Carboplatin, etc.); 8) monoclonal antibodies conjugated with anticancer drugs, toxins, and/or radionuclides, etc.; 9) biological response modifiers (e.g., interferons [e.g., IFN-.alpha., etc.] and interleukins [e.g., IL-2, etc.], etc.); 10) adoptive immunotherapy; 11) hematopoietic growth factors; 12) agents that induce tumor cell differentiation (e.g., all-trans-retinoic acid, etc.); 13) gene therapy techniques; 14) antisense therapy techniques; 15) tumor vaccines; 16) therapies directed against tumor metastases (e.g., Batimistat, etc.); and 17) inhibitors of angiogenesis.

In other embodiments, the therapeutic regimen is administration of a vaccine directed to a protein encoded by the hypomethylated gene. Vaccines may be directed to one or more of the target genes identified herein. For example, NY-ESO-1 and MAGEA3 are currently undergoing clinical trials in various human malignancies, including NSCLC (see, Hirschowitz, E. A., et al., J Thorac Oncol 1: 93-104 (2006); Karanikas, V. et al., Cancer Biol Ther 7 (2007); Raez, L. E., et al., Expert Opinion on Emerging Drugs 11: 445-459 (2006); and Old, L. J., Cancer Immun 8: Suppl 1, 1 (2008)). In certain embodiments, vaccines are directed to multiple targets found to be coordinately overexpressed. Vaccines directed to the hypomethylated gene can be made by methods well-known in the art (see e.g., Davis et al., Proc Natl Acad Sci USA. 101(29): 10697-10702, 2004).

The materials for use in the methods of the invention are ideally suited for the preparation of a kit. Such a kit may comprise a carrier device containing one or more containers such as vials, tubes, and the like, each of the containers comprising one of the separate elements to be used in the method. The kit may contain reagents, as described above for differentially modifying methylated and non-methylated cytosine residues. One of the containers may include a probe which is or can be detectably labeled. Such probe may be a nucleic acid sequence specific for a promoter region associated with a gene selected from the group consisting of TKTL1, H19, MAGEA2, MAGEA3, MAGEA4, MAGEA11, GPR17, GRIN1, C19ORF28, MAGEA12, MAGEA1, MAGEA5, NY-ESO-1, MAGEA9, MAGEA6, MAGEB2, CT45-2, SBSN, G6PD, ZNF711, CrispL, KRT86, KIPV467, KRT81, CSPG5, PP1R14A, KISS1R, KIAA1937 protein, SOX30, DEAD, and KBGP. For example, oligonucleotide probes of the invention can be included in a kit and used for detecting the presence of hypomethylated nucleic acid sequences in a sample containing a nucleic acid sequence of the genes TKTL1, H19, MAGEA2, MAGEA3, MAGEA4, MAGEA11, GPR17, GRIN1, C19ORF28, MAGEA12, MAGEA1, MAGEA5, NY-ESO-1, MAGEA9, MAGEA6, MAGEB2, CT45-2, SBSN, G6PD, ZNF711, CrispL, KRT86, KIPV467, KRT81, CSPG5, PP1R14A, KISS1R, KIAA1937 protein, SOX30, DEAD, and/or KBGP. The kit may also include a container comprising a reporter, such as an enzymatic, fluorescent, or radionucleotide label to identify the detectably labeled oligonucleotide probe.

In certain embodiments, the kit utilizes nucleic acid amplification in detecting the target nucleic acid. In such embodiments, the kit will typically contain both a forward and a reverse primer for each target gene. Such oligonucleotide primers are based upon identification of the flanking regions contiguous with the target nucleotide sequence. Accordingly, the kit may contain primers useful to amplify and screen a promoter region of a gene selected from the group consisting of TKTL1, H19, MAGEA2, MAGEA3, MAGEA4, MAGEA11, GPR17, GRIN1, C19ORF28, MAGEA12, MAGEA1, MAGEA5, NY-ESO-1, MAGEA9, MAGEA6, MAGEB2, CT45-2, SBSN, G6PD, ZNF711, CrispL, KRT86, KIPV467, KRT81, CSPG5, PP1R14A, KISS1R, KIAA1937 protein, SOX30, DEAD, and KBGP. If there is a sufficient region of complementarity, e.g., 12, 15, 18, or 20 nucleotides, then the primer may also contain additional nucleotide residues that do not interfere with hybridization but may be useful for other manipulations. For example, such other residues may be sites for restriction endonuclease cleavage, for ligand binding or for factor binding or linkers or repeats. The oligonucleotide primers may or may not be such that they are specific for modified methylated residues. The kit may optionally contain oligonucleotide probes. The probes may be specific for sequences containing modified methylated residues or for sequences containing non-methylated residues. The kit may optionally contain reagents for modifying methylated cytosine residues. The kit may also contain components for performing amplification, such as a DNA polymerase and deoxyribonucleotides. Means of detection may also be provided in the kit, including detectable labels on primers or probes. Kits may also contain reagents for detecting gene expression for one of the markers of the present invention. Such reagents may include probes, primers, or antibodies, for example. In the case of enzymes or ligands, substrates or binding partners may be used to assess the presence of the marker. In particular embodiments, the kit may include one or more primers or primer pairs selected from the sequences set forth in SEQ ID NOs: 1-58.

The following examples are intended to illustrate but not limit the invention.

### Example 1

### Activation of Genes Via Promoter Demethylation in Head and Neck Cancer and Lung Cancer

In this example, an integrative method was used to analyze expression in primary head and neck squamous cell carcinoma (HNSCC) and pharmacologically demethylated cell lines to identify aberrantly demethylated and expressed candidate proto-oncogenes and cancer testes antigens in HNSCC. HNSCC is useful as a solid tumor model system, due to the established role of epigenetic changes in its pathogenesis, as well as the availability of normal, minimally transformed cell lines for use in gene discovery strategies. Using pharmacologic demethylation in normal, minimally-transformed oral keratinocyte cell lines combined with Cancer Outlier Profile Analysis (COPA) in primary tissues as a discovery approach, a set of candidate proto-oncogenes that undergo aberrant demethylation and increased expression in primary human tumors were identified.

Functional data suggest that expression of these genes is associated with tumor promotion. Additional analyses demonstrated promoter homology and coordinated upregulation in individual tumors for subsets of these target genes (proto-oncogenes). Coordinated promoter demethylation and simultaneous transcriptional upregulation of proto-oncogene candidates with promoter homology, and phylogenetic footprinting of these promoters demonstrated potential recognition sites for the transcription factor BORIS were noted. Aberrant BORIS expression correlated with upregulation of candidate proto-oncogenes in multiple human malignancies including primary non-small cell lung cancers and HNSCC, induced coordinated proto-oncogene specific promoter demethylation and expression in non-tumorigenic cells, and transformed NIH3T3 cells. Coordinated, epigenetic unmasking of multiple genes with growth promoting activity occurred in aerodigestive cancers, and BORIS was implicated in the coordinated promoter demethylation and reactivation of epigenetically silenced genes in human cancers.

Histopathology. All samples were analyzed by the Pathology department at Johns Hopkins Hospital. Tissues were obtained via Johns Hopkins Institutional Review Board approved protocols under JHM IRB Protocol #92-07-21-01, “Detection of Genetic Alterations in Head and Neck Tumors.” Normal samples were microdissected and DNA prepared from the mucosa. Tumor samples were confirmed to be head and neck squamous and subsequently microdissected to separate tumor from stromal elements to yield at least 80% tumor cells. Tissue DNA was extracted as described below.

5Aza-dC and TSA Treatment of Cells. These in vitro techniques employed treatment of cultured cells with 5-aza-deoxycytidine (5Aza-dC, a cytosine analog which cannot be methylated) with or without Trichostatin A (TSA, a histone deacetylase inhibitor) and subsequent expression array analysis with validation of tumor suppressor gene targets. HNSCC cell lines were treated with 5Aza-dC and/or TSA as described previously. Briefly, cells were split to low density (1×10⁶ cells/T-75 flask) 24 hours before treatment. Stock solutions of 5Aza-dC (Sigma, St. Louis, Mo.) and ISA (Sigma) were dissolved in DMSO (Sigma) and 100% ethanol, respectively. Cells were treated with 5 μM 5-Aza-deoxycytidine for 5 days and 300 nmol/L TSA for the last 24 hours. Baseline expression was established by mock-treated cells with the same volume of DMSO or ethanol. Two normal oral keratinocyte cell lines (OKF6-Tert1 and OKF6-Tert1Q, immortalized with hTert), were treated in duplicate by 5-aza-deoxycytidine/trichostatin A.

Oligonucleotide microarray analysis and QRT-PCR analysis. Total cellular RNA was isolated using the RNEASY RNA isolation kit (Qiagen, Valencia, Calif.) according to the manufacturer's instructions. Oligonucleotide microarray analysis was carried out using the GENECHIP U133plus2 Affymetrix expression microarray (Affymetrix, Santa Clara, Calif.). Samples were converted to labeled, fragmented, cRNA per the manufacturer's protocol for use on the expression microarray. Signal intensity and statistical significance were established for each transcript using dChip version 2005. 2-fold increase based on the 90% confidence interval of the result and expression minus baseline >50 was used as the statistical cutoff value after 5Aza-dC and/or TSA treatment to identify upregulated candidate genes.

Public datasets. The public databases used in this study were the University of California Santa Cruz (UCSC) Human Genome reference sequence and the annotation database from the May 2004 freeze (hg17). 56 HNSCC expression microarrays were obtained from public datasets from Oncomine (Oncomine.org, Ann Arbor, Mich.). 14 expression microarrays that had been previously studied from the same platform were incorporated and all microarrays were normalized for COPA analysis. Also utilized were the expO datasets (1185 tumors on the Affymetrix U133plus2 mRNA expression platform) available online as part of the Gene Expression Omnibus (GEO/NCBI). These data are publicly available online as part of the Gene Expression Omnibus (GEO/NCBI), produced by the International Genomics Consortium. This analysis utilized expression array data for 47,000+ genes measured in 1041 human tumors of various histologies.

Cancer outlier profile analysis (COPA). COPA was applied to a cohort of 68 tissues (49 tumors, 19 normals), with each gene expression data set containing 14,500 probe sets. Briefly, gene expression values were median centered, setting each gene's median expression value to zero. The median absolute deviation (MAD) was calculated and scaled to 1 by dividing each gene expression value by its MAD. Of note, median and MAD were used for transformation as opposed to mean and standard deviation so that outlier expression values did not unduly influence the distribution estimates, and were thus preserved post-normalization. Finally, the 75th, 90th, and 95th percentiles of the transformed expression values were calculated for each gene and then genes were rank-ordered by their percentile scores, providing a prioritized list of outlier profiles. For the purposes of a rank-list, the 90^{th} percentile was chosen based on sample-size analysis (49 tumors, 19 normals). For details of the method refer to Tomlins et. al. (Science 310:644-648, 2005).

Integrative epigenetics. Target genes from the Affymetrix U133A mRNA expression microarray platform were ranked by COPA upregulation at the 90^{th} percentile (from 49 tumors and 19 normal tissues). The U133A microarray platform (Affymetrix, Santa Clara Calif.) has approximately 14,500 probe sets. A second rank list was produced by ranking genes in descending order of the degree of upfold regulation upon 5-aza/TSA treatment. These two sources of information (gene set demonstrating upregulation with 5-aza) and COPA score were combined by using a rank product. These two rankings were combined to rank all targets and permutation of the data was used to establish significance with a threshold of <0.005. This resulted in 106 genes deemed significant. The top 26 of these targets were comprehensively evaluated. Presence of CpG islands in these genes was determined by MethPrimer. In order to not exclude genes outside the U133A platform, also considered were all other genes in the U133plus2 platform on the sole basis of 5-aza/TSA upfold regulation. For all genes which did not have tissue mRNA expression array information amenable to COPA analysis, only statistically significant reexpression after 5-aza treatment was considered. 45 genes were studied that had an experimental versus baseline expression (EB)>2.0, based on the 90% confidence interval and E−B>50. All genes were then studied for the presence of CpG islands in promoters or the first intron. Initially, an in silk° approach was used to confirm the presence of a CpG island using the UCSC genome browser which relies on GC content of >50%, >200 bp, >0.6 observed to expected CG's.

DNA extraction. Samples were centrifuged and digested in a solution of detergent (sodium dodecylsulfate) and proteinase K, for removal of proteins bound to the DNA. Samples were first purified and desalted with phenol/chloroform extraction. Digested sample was subjected twice to ethanol precipitation, and subsequently resuspended in 500 μL of LoTE (EDTA 2.5 mmol/L and Tris-HCl 10 mmol/L) and stored at −80° C.

Bisulfite treatment. DNA from salivary rinses was subjected to bisulfite treatment, as described previously. In short, 2 μg of genomic DNA was denatured in 0.2 M of NaOH for 30 minutes at 50° C. This denatured DNA was then diluted into 500 μL of a solution of 10 mmol/L hydroquinone and 3 M sodium bisulfite. This was incubated for 3 hours at 70° C. After, the DNA sample was purified with a SEPHAROSE column (WIZARD DNA Clean-Up System; Promega, Madison, Wis.). Eluted DNA was treated with 0.3 M of NaOH for 10 minutes at room temperature, and precipitated with ethanol. This bisulfite-modified DNA was subsequently resuspended in 120 μL of LoTE (EDTA 2.5 mmol/L and Tris-HCl 10 mmol/L) and stored at −80° C.

Bisulfite sequencing. Bisulfite sequence analysis was performed to check the methylation status in primary tumors and normal tissues, as well as in cell lines. Bisulfite-treated DNA was amplified using primers designed using the MethPrimer program (Li and Dahiya, Bioinforinatics 18(11):1427-31, 2002) to span areas of CpG islands in the promoter or first intron. Primer sequences were designed to not have CG dinucleotides (see Table 1 below for primer sequences). The PCR products were gel-purified using the QIAQUICK gel extraction kit (Qiagen), according to the manufacturer's instructions. Each amplified DNA sample was applied with nested primers to the Applied Biosystems 3700 DNA analyzer using BD terminator dye (Applied Biosystems, Foster City, Calif.).

Quantitative unmethylation-specific PCR (QUMSP). To selectively amplify demethylated promoter regions in genes of interest, probe and primers were designed using data from bisulfite sequencing of primary tumors which are complimentary only to bisulfite-converted sequences known to be demethylated in tumor (see Table 1). Primer combinations were validated using in vitro methylated and demethylated controls.

TABLE 1
QUMSP Primers

| | | SEQ |
|---|---|---|
| Target | | ID |
| Gene | | NO: |
| | Forward Primers | |
| MageA2B-QU | GTTGTGAATTTAGGGAAGTTATGG | 1 |
| MageA3-QU | ATTATTTTATTTTGTTGATTTTTGTTGT | 3 |
| MageA4-QU | TAAAAATTTGTTTTTTTTATTGA | 5 |
| Magea11-QU | AGTAGATATTATTGGTAGAGGATGG | 7 |
| GPR17-QU | TTATTTCGTGAGTAAGATTTTTCGG | 9 |
| GRIN1-QU | TTTTTTAAGTTAGGGAGGGGTATGT | 11 |
| H19-QU | TTTAGAGTTTGTGGTTAAGGTGG | 13 |
| C19ORF28- | TGTATATTGTTTGTGGGTTATGA | 15 |
| QU | | |
| TKTL1-QU | TGTGTAGAGAAAGAAGATTTTGTATTTGT | 17 |
| | Reverse Primers | |
| MageA2B-QU | CATCAAACCATTACTCAAAACAAA | 2 |
| MageA3-QU | CTCAAAACTCACATACCTAACCAC | 4 |
| MageA4-QU | ATAATACAAAATTATTTAAAATCATC | 6 |
| Magea11-QU | CTTCCCTAAATTTACAACAAAAACG | 8 |
| GPR17-QU | GACTATCATAACAACAAACGACGC | 10 |
| GRIN1-QU | AAACACCAAAAATCACTAAAACACC | 12 |
| H19-QU | AACAAAAAAATCATATAAAAAAAACATA | 14 |
| C19ORF28- | CAAAATCTCAAAAAAACTACCCAAC | 16 |
| QU | | |
| TKTL1-QU | CCCAATACATTTATATAATATCAAT | 18 |

qRT-PCR. Total RNA was measured and adjusted to the same amount for each cell line, and then cDNA synthesis was performed using oligo-dt with the SUPERSCRIPT first-strand DNA synthesis kit (Invitrogen). The final cDNA products were used as the templates for subsequent PCR with primers designed specifically for each candidate gene (see Table 2). GAPDH was examined to ensure accurate relative quantitation in qRT-PCR. qRT-PCR heat maps were generated by median-normalization by gene, logged and heat maps generated using Excel.

TABLE 2
qRT-PCR Primers

| | | SEQ | | SEQ |
|---|---|---|---|---|
| Target | | ID | | ID |
| Gene | Forward Primer | NO: | Reverse Primer | NO: |
| magea2 | ATCTGCCTGTGGGTCTTCAT | 19 | GAAGAGGAAGAAGCGGTCTG | 20 |
| magea3 | AAGATCTGCCAGTGGGTCTC | 21 | CCCCAGGGTGACTTCAACTA | 22 |
| magea4 | AGGAGAAGATCTGCCTGTGG | 23 | CAGCCTCCTGCTCCTCAGTA | 24 |
| boris | cacattcgtacccacactgg | 25 | tcccctgatccacacttctc | 26 |
| grin1 | gtcctccaaagacacgagca | 27 | ccgtctgtctgtctgtctgc | 28 |
| tktl1 | gatcactacccgcaaggtg | 29 | cacagggtaaagaggccaaa | 30 |
| h19 | gagctctcaggagggaggat | 31 | agacctggcctcgtctcc | 32 |
| c19orf28 | acccttgcccctcagagc | 33 | ggccacagcaggaggcta | 34 |
| gpr17 | aactctcaggctctgactcca | 35 | gccagggtattgccaactaa | 36 |
| magea11 | gaggagaacaagtgctgtgg | 37 | agcagcaggcaactcctcta | 38 |

Transfection of human expression vectors. A full-length ORF cDNA of MAGEA2B, MAGEA4, H19, TKTL1 were obtained for transient transfections. Cell lines were plated at 2×10⁵/well using 6-well plates and transfected with either empty vector or gene of interest using the FUGENE 6 transfection reagent (Roche, Basel, Switzerland) according to the manufacturer's protocol. Calcein florescence was measured by the Spectramax M2e 96-well fluorescence plate reader Molecular Devices (Sunnyvale, Calif.). Live cells are distinguished by the presence of ubiquitous intracellular esterase activity, determined by the enzymatic conversion of the virtually nonfluorescent cell-permeable calcein AM to the intensely fluorescent calcein. The polyanionic calcein dye is well retained within live cells, producing an intense uniform green fluorescence (excitation/emission ˜495 nm/515 nm).

Anchorage-independent growth assay. Soft agar assays were conducted after transfection of cells with mammalian expression vectors. Cells were counted and approximately 5000 were added into each 6-well plate. The bottom layer was composed of 0.5% agar, DMEM+10% FBS, plus additives, while the cells were suspended in a top layer of 0.35% agar, DMEM+10% FBS, plus additives. BORIS Inducible promoter constructs were incubated in the presence of low doxycycline (0.01 mg/mL). Soft agar assays were incubated at 37° C. for 2 weeks.

Statistical analysis. The QUMSP data was analyzed using a Wilcoxon-Mann-Whitney rank test. The p-values were corrected using the Benjamini-Hochberg procedure, and significance was defined as p_{corr}<0.05. We looked for similarities in the methylation patterns between genes by performing an analysis of correlations between QUMSP readings on the genes across all samples. 1000 permutations of the samples were used to establish significance, with α=0.05. For the expression data, the normalized data was log-transformed and correlation analysis was performed across all samples between each of the genes in the study. Significance was determined by assuming a normal distribution in the log-transformed expression levels and applying Student's t-distribution with an alpha of 0.05. All analyses were performed using Matlab. Comparisons of promoter homology were done with European Bioinformatics Institutes' ClustalW sequence alignment and phylogram software and the PromoterWise application.

Integrative Discovery of Epigenetically Unmasked Genes in HNSCC. We hypothesized that normal cell lines contain methylated genes that are typically repressed in normal tissues, but that these genes can be re-expressed by pharmacologic manipulation. A subset of these genes would include candidate proto-oncogenes activated by demethylation in human cancers that could be further selected on the basis of primary tumor expression array analysis using integrative methods. Methods of epigenetic screening using 5-aza/TSA treatment that have been found to be successful in defining candidate tumor suppressor genes were adapted for the present study. Two TERT-transformed normal oral keratinocyte cell lines were treated with 5 μM 5-aza deoxycytidine for four days and Trichostatin A for one day prior to harvesting total RNA for expression array analysis using dChip.

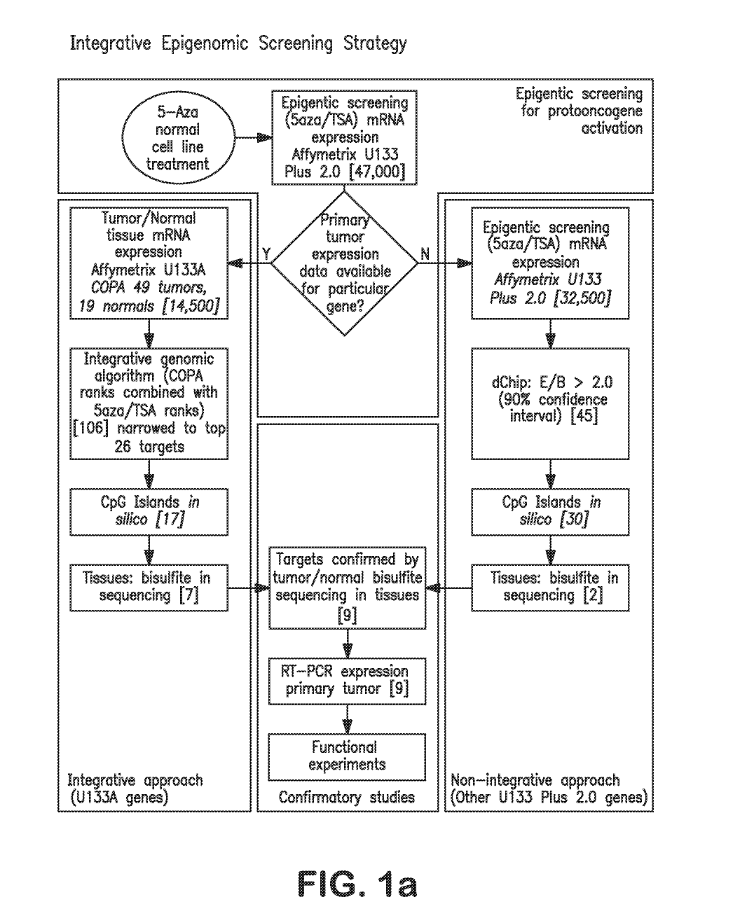

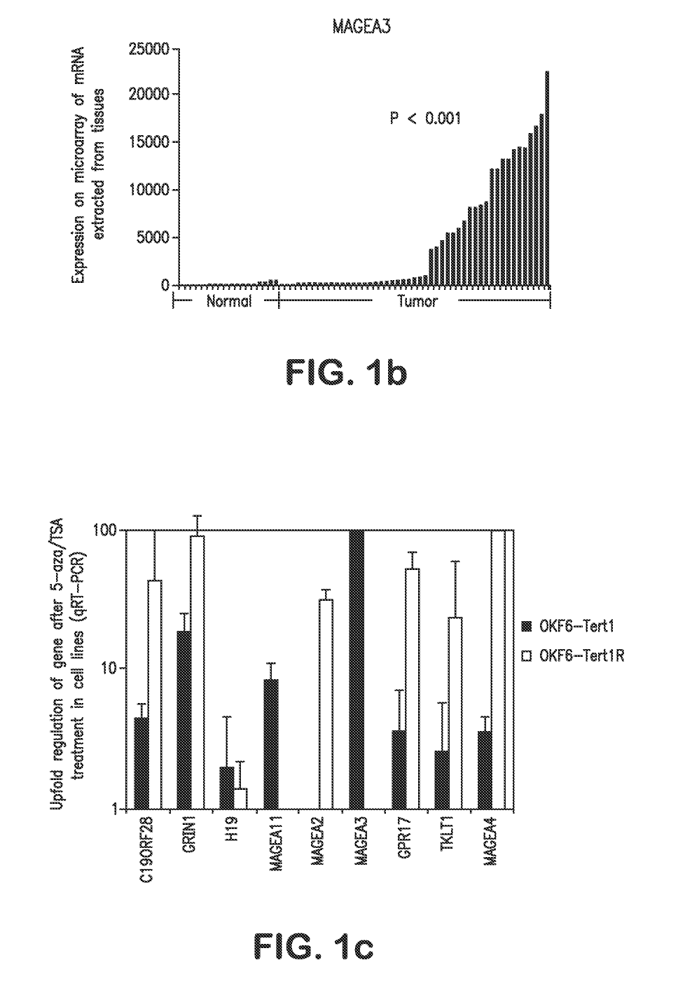

Concurrently, a comparative epigenetic approach utilizing Cancer Outlier Profiling Analysis (COPA) was performed using 49 primary HNSCC and 19 normal mucosal tissues assayed for mRNA expression on the Affymetrix U133A mRNA expression microarray platform (16,383 probe sets) compiled from prior work and public sources of expression (oncomine.org). COPA is particularly useful to determine differences in expression for particular genes in subsets of primary tumor samples, with improved performance compared to statistical tools that rely on median or average expression difference between two datasets (Tomlins et al., Science 310:644-8, 2005). COPA was calculated at the 90^{th} percentile for the final rankings of all 16,383 features of the arrays, as this resulted in the most pronounced differences in expression with this sample size. Statistical significance of the expression differences in the COPA diagrams was measured by Mann-Whitney U test (FIG. 1B).

Gene ranks were determined in two ways: 1) COPA ranking at the 90^{th} percentile of upregulation in primary tumor tissue versus normal tissue expression, and 2) fold upregulation after pharmacologic demethylation after dChip normalization in cell lines. An integrative rank product was calculated (FIG. 1A). Using a significance threshold (α=0.005) and subsequent random permutation of the rank-lists, 106 genes were identified that were significantly differentially upregulated based on epigenetic screening and tissue microarray expression (Table 3). The top scoring 26 genes were selected for further analyses. Seventeen of 26 genes contained promoter-associated CpG islands as identified utilizing the MethPrimer software and were selected for further studies. In a separate parallel analysis to account for possible activated proto-oncogenes not included in the U133A platform, 32,500 genes were analyzed in the U133plus2 platform ranked on the sole basis of 5-aza/TSA fold upregulation in the normalized cell lines that were not included in primary tumor expression array analysis. 45 target genes were identified with >2-fold upregulation at 90% confidence interval and an average difference value expression over baseline greater than 50. Among these, 30 were confirmed to have CpG islands (Table 4).

TABLE 3

| Gene | Accession | Desc | FoldChang [symbol] | AzaRank | COPASco [symbol] | [symbol] 90DiffNor [symbol] | OverallRank |
|---|---|---|---|---|---|---|---|
| melanoma antigen family A, 4 [symbol] | AW438674 | gb:AW438 [symbol] | 18.04 | 13 | 7 | 39.62488 | 1 |
| melanoma antigen family A, 6 | U10691 | gb:U10691 | 4.55 | 168 | 4 | 57.21295 | 2 |
| melanoma antigen family A, 3 | BC000340 | gb:BC000 | 2.94 | 539 | 3 | 57.5791 | 3 |
| melanoma antigen family A, 2 | U82671 | gb:U82671 | 1.91 | 2148 | 1 | 160.0283 | 4 |
| chemokine (C-C motif) ligand | NM_004591 | gb:NM_00 | 27.75 | 7 | 318 | 5.489771 | 5 |
| dehydrogenase/reductase (SD | AK000345 | gb:AK0003 | 78.58 | 3 | 1024 | 3.408703 | 6 |
| oncostatin M | BG437034 | gb:BG437 | 6.99 | 71 | 45 | 14.6672 | 7 |
| melanoma antigen family A, 1 | BC003408 | gb:BC0034 | 3.59 | 308 | 15 | 25.73548 | 8 |
| glutamate receptor, ionotropic | NM_007327 | gb:NM_00 | 16.43 | 15 | 380 | 5.096238 | 9 |
| interleukin 8 | AF043337 | gb:AF0433 | 6.26 | 88 | 117 | 8.772762 | 10 |
| interleukin 6 (interferon, beta 2 | NM_000600 | gb:NM_00 | 6.33 | 86 | 138 | 8.153837 | 11 |
| transketolase-like 1 | Z49258 | gb:Z49258 | 56.88 | 6 | 2001 | 2.503557 | 12 |
| enolase 2 (gamma neuronal) | NM_001975 | gb:NM_00 | 7.6 | 59 | 216 | 6.399704 | 13 |
| fatty acid binding protein 4, ad | NM_001442 | gb:NM_00 | 5.54 | 107 | 128 | 8.483477 | 14 |
| immunoglobulin heavy consta | U80139 | gb:U80139 | 4.6 | 162 | 88 | 10.4607 | 15 |
| omithine decarboxylase 1 | NM_002539 | gb:NM_00 | 4.66 | 155 | 96 | 9.695386 | 16 |
| dehydrogenase/reductase (SD | NM_005794 | gb:NM_00 | 20.44 | 11 | 1363 | 3.037984 | 17 |
| chemokine (C-X-C motif) ligan | AF002985 | gb:AF0029 | 2.73 | 671 | 23 | 21.87494 | 18 |
| melanoma antigen family A, 1 | BC004479 | gb:BC0044 | 2.7 | 690 | 24 | 19.81325 | 19 |
| lymphocyte-specific protein ty | NM_005356 | gb:NM_00 | 5.38 | 116 | 147 | 7.868405 | 20 |
| cytokeratin type II | NM_004693 | gb:NM_00 | 4.55 | 166 | 111 | 8.994016 | 21 |
| G protein-coupled receptor 17 | NM_005291 | gb:NM_00 | 2.8 | 622 | 37 | 15.99263 | 22 |
| cytoplasmic FMR1 interacting | AL161999 | gb:AL1619 | 5.89 | 97 | 248 | 5.967384 | 23 |
| matrix metalloproteinase 13 ( | NM_002427 | gb:NM_00 | 4.03 | 226 | 134 | 8.264698 | 24 |
| nuclear receptor subfamily 4, | D49728 | gb:D49728 | 6.38 | 85 | 374 | 5.137292 | 25 |
| chemokine (C-X-C motif) ligan | AF030514 | gb:AF0305 | 2.26 | 1191 | 28 | 18.11945 | 26 |
| dipeptidylpeptidase 4 (CD26, | M80536 | gb:M8053 | 2.46 | 914 | 40 | 15.33985 | 27 |
| melanoma antigen family B, 2 | NM_002364 | gb:NM_00 | 3.92 | 247 | 158 | 7.682364 | 28 |
| early growth response 4 | NM_001965 | gb:NM_00 | 2.26 | 1190 | 33 | 16.85125 | 29 |
| keratin, hair, basic, 1 | NM_002281 | gb:NM_00 | 25.69 | 8 | 5537 | 0.431888 | 30 |
| baculoviral IAP repeat-containi | U37546 | gb:U37546 | 6.66 | 77 | 596 | 4.27014 | 31 |
| gb:BC006164.1/DB_XREF = gi | BC006164 | gb:BC006 | 3.88 | 256 | 181 | 7.056746 | 32 |
| apolipoprotein C-I | NM_001645 | gb:NM_00 | 3.9 | 251 | 193 | 6.75713 | 33 |
| dickkopf homolog 1 (Xenopus | NM_012242 | gb:NM_01 | 2.93 | 543 | 95 | 9.819803 | 34 |
| armadillo repeat containing, X | NM_014782 | gb:NM_01 | 3.43 | 353 | 160 | 7.640024 | 35 |
| bone marrow stromal cell anti | NM_004335 | gb:NM_00 | 6.67 | 76 | 767 | 3.858544 | 36 |
| fatty acid binding protein 6, ile | U19869 | gb:U19869 | 2.19 | 1333 | 54 | 13.53217 | 37 |
| calbindin 1, 28 kDa | NM_004929 | gb:NM_00 | 1.91 | 2136 | 34 | 16.70063 | 38 |
| DnaJ (Hsp40) homolog, subfa | AV729634 | gb:AV729 | 4.63 | 160 | 511 | 4.530018 | 39 |
| lysosomal associated multisp | NM_006762 | gb:NM_00 | 7.31 | 63 | 1432 | 2.978136 | 40 |
| lysosomal associated multisp | AI589086 | gb:AI5890 | 5.46 | 110 | 873 | 3.672105 | 41 |
| early growth response 2 (Krox | NM_000399 | gb:NM_00 | 4.27 | 194 | 497 | 4.604386 | 42 |
| transglutaminase 2 (C polype | AL031651 | gb:AL0316 | 5.91 | 96 | 1026 | 3.407147 | 43 |
| homeo box HB9 | AI738662 | gb:AI7386 | 11.84 | 27 | 3683 | 1.386694 | 44 |
| interferon alpha-inducible pro | NM_005532 | gb:NM_00 | 6.13 | 89 | 1174 | 3.210361 | 45 |
| nuclear receptor subfamily 4, | S77154 | gb:S77154 | 2.81 | 612 | 180 | 7.059269 | 46 |
| myosin light polypeptide kina | NM_005965 | gb:NM_00 | 2.69 | 699 | 166 | 7.437435 | 47 |
| nucleoporin 210 kDa | AI867102 | gb:AI8671 | 4.27 | 195 | 622 | 4.160394 | 48 |
| interleukin 8 | NM_000584 | gb:NM_00 | 4.22 | 209 | 592 | 4.279749 | 49 |
| Full length cDNA clone CS0D | N30878 | gb:N30878 | 2.43 | 951 | 131 | 8.327486 | 50 |
| insulin-like 3 (Leydig cell) | AI991694 | gb:AI9916 | 3.96 | 243 | 538 | 4.462851 | 51 |
| chitinase 3-like 1 (cartilage gly | M80927 | gb:M8092 | 2.3 | 1126 | 119 | 8.710771 | 52 |
| pentraxin-related gene, rapidly | NM_002852 | gb:NM_00 | 2.12 | 1478 | 98 | 9.635762 | 53 |
| chemokine (C-X-C motif) ligan | NM_002993 | gb:NM_00 | 2.03 | 1725 | 87 | 10.51205 | 54 |
| carboxyl ester lipase (bile salt | NM_001807 | gb:NM_00 | 2.71 | 685 | 227 | 6.267059 | 55 |
| oxidised low density lipoprotei | AF035776 | gb:AF0357 | 2.2 | 1310 | 121 | 8.607828 | 56 |
| nuclear receptor subfamily 4, | NM_002135 | gb:NM_00 | 2.97 | 524 | 306 | 5.549491 | 57 |
| ATP-binding cassette, sub-fa | U88667 | gb:U88667 | 2.77 | 639 | 251 | 5.947201 | 58 |
| CD74 antigen (invariant polyp | K01144 | gb:K01144 | 5.62 | 104 | 1549 | 2.8797 | 59 |
| glyceral kinase | NM_000167 | gb:NM_00 | 3.38 | 371 | 448 | 4.783837 | 60 |
| chorionic gonadotropin, beta p | NM_000737 | gb:NM_00 | 1.96 | 1956 | 89 | 10.42807 | 61 |
| chemokine (C-X-C motif) ligan | NM_001511 | gb:NM_00 | 3.07 | 478 | 365 | 5.200393 | 62 |
| neurofilament 3 (150 kDa medi | NM_005382 | gb:NM_00 | 3.96 | 242 | 721 | 3.935926 | 63 |
| matrix metalloproteinase 9 (g | NM_004994 | gb:NM_00 | 3.62 | 299 | 619 | 4.167812 | 64 |

[symbol]

indicates data missing or illegible when filed

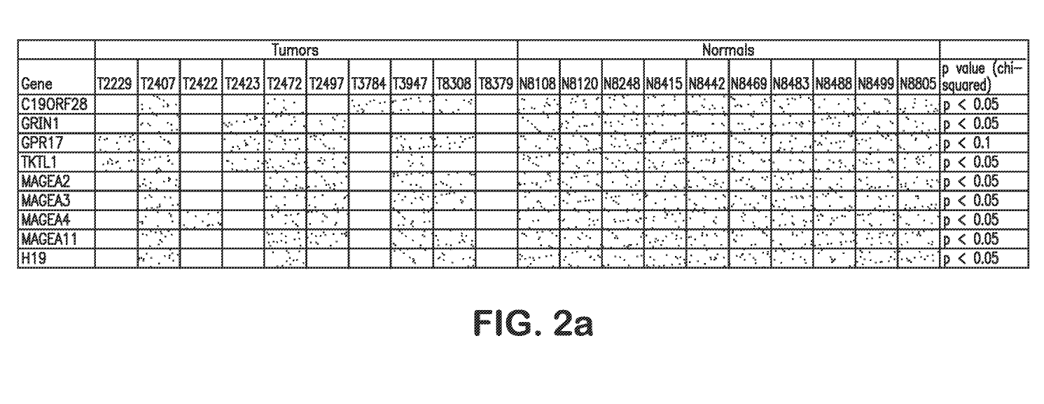

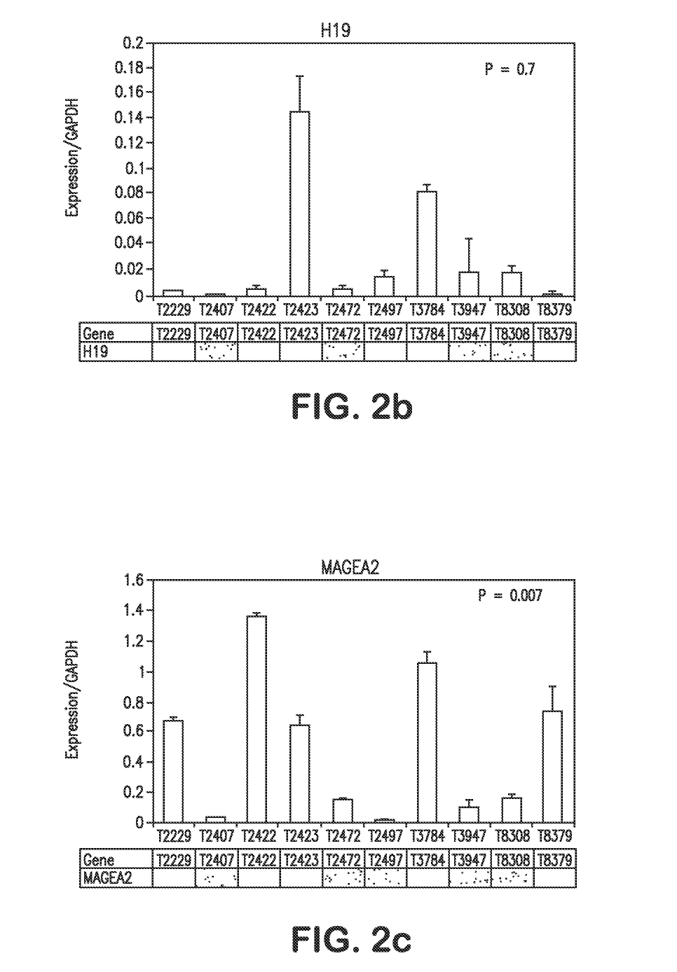

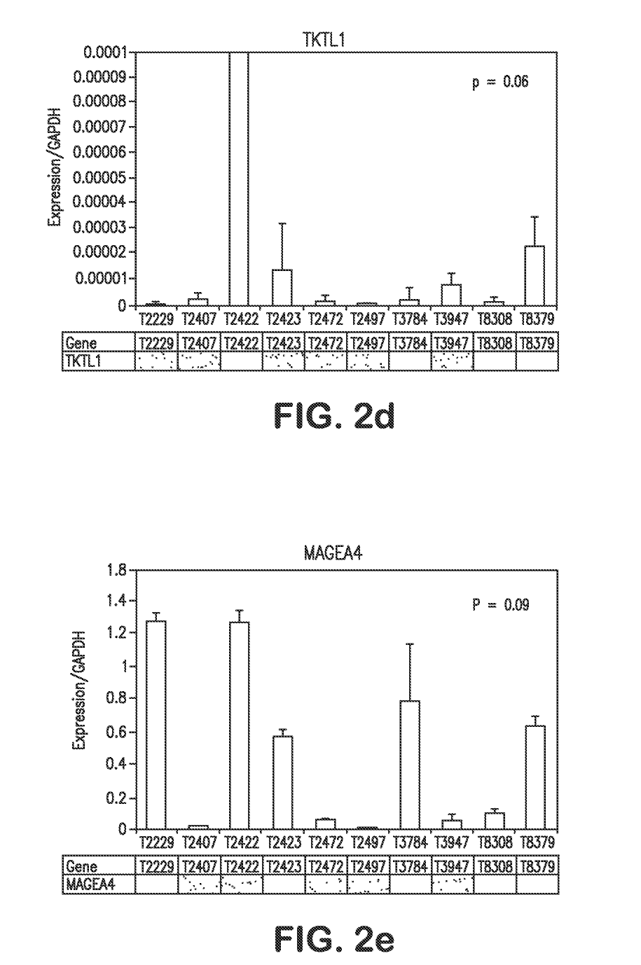

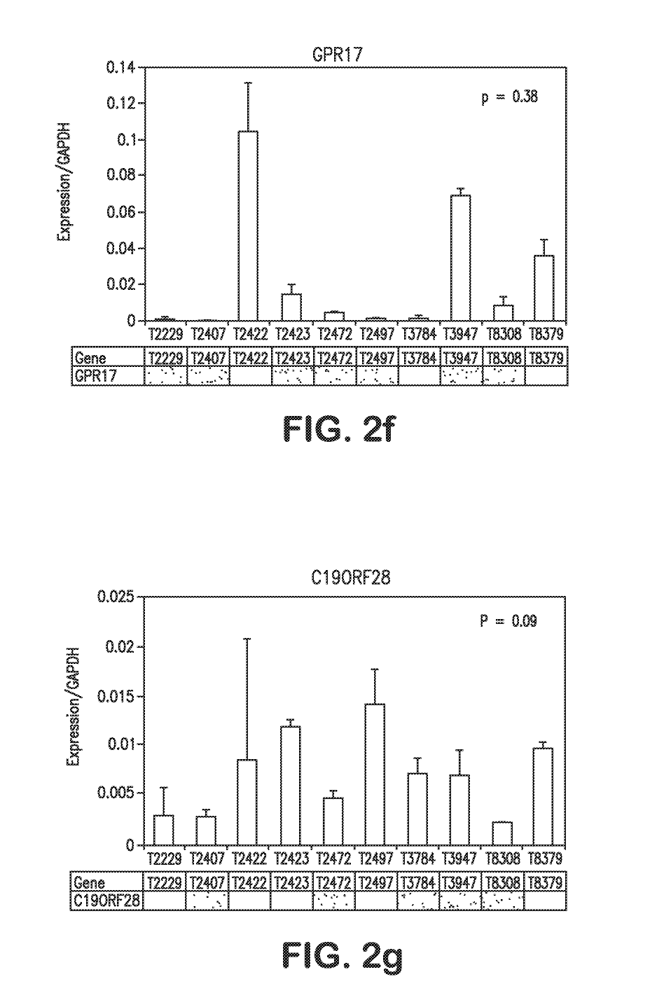

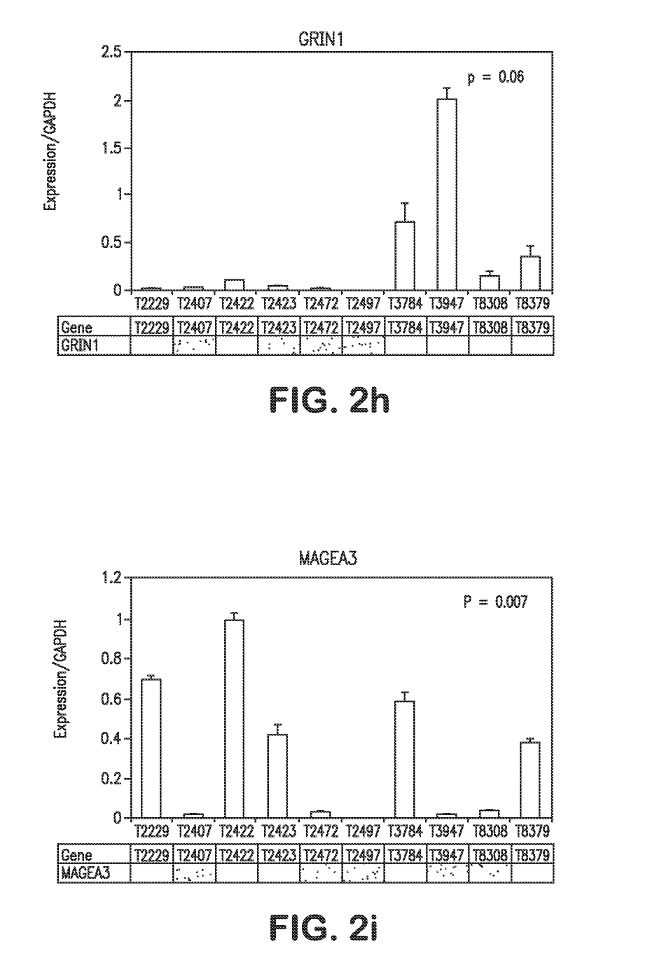

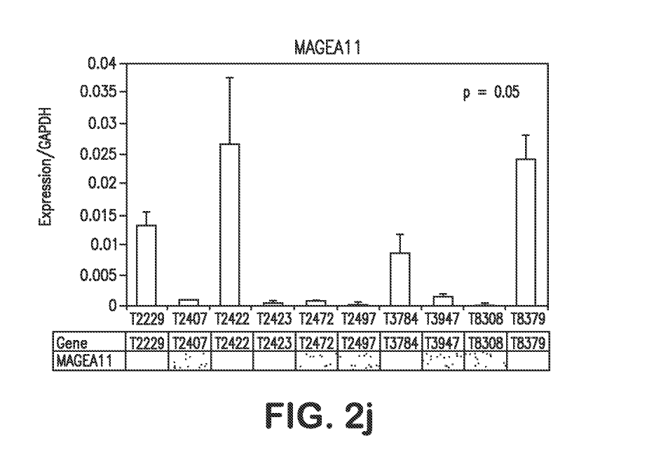

Validation of tumor specific promoter demethylation of target genes. CpG islands in the promoter region of the 47 selected gene targets with CpG islands were bisulfite sequenced in normal mucosal samples from patients without a cancer diagnosis to confirm epigenetic silencing in mature upper aerodigestive tract mucosa (Table 4). Only 18/47 promoter regions demonstrated complete methylation at all sequenced CpG sites in all normal tissues. These targets were subsequently bisulfite sequenced in 10 primary HNSCC to assay for the presence of hypomethylation. (FIG. 2A). Of these remaining targets, 9/18 showed demethylation in tumor tissues in greater than 30% of the samples, including TKTL1 (4/10, p<0.05), H19 (6/10, p<0.05), MAGEA2 (5/10, p<0.05), MAGEA3 (5/10, p<0.05), MAGEA4 (5/10, p<0.05), MAGEA11 (5/10, p<0.05), GPR17 (3/10, p<0.10), GRIN1 (6/10, p<0.05), C19ORF28 (5/10, p<0.05), (chi-squared). To confirm transcriptional upregulation of target genes with 5-aza/TSA treatment in the cell line system, quantitative RT-PCR was performed on 5-aza/TSA-treated normal cells compared to mock-treated cells for these nine genes (FIG. 1C). Each gene demonstrated significant upregulation by 5-aza/TSA treatment in at least one cell line supporting functional gene regulation by promoter hypomethylation. Using the initial cohort of 10 primary tumors, a preliminary analysis was performed to determine the relationship of promoter hypomethylation to expression. QRT-PCR expression with the bisulfite sequencing of the respective promoter below is shown in FIG. 2B-J. The Mann Whitney U test was employed to compare QRT-PCR expression of the methylated and unmethylated groups. Three genes had statistically significant increased expression in the unmethylated group: MAGEA2 (p=0.007), MAGEA3 (p=0.007), MAGEA11 (p=0.05). Possible associations between expression and promoter methylation status in this small cohort were also suggested for TKTL1 (p=0.06), MAGEA4 (p=0.09), C19ORF28 (p=0.09), GRIN1 (p=0.06), yet H19 (p=0.7) but GPR17 (p=0.38) did not show this association.

TABLE 4

| Probe_set | Accession | Name | CpG Insland | Meth Nls | Meth Tumoc | Fold Change |
|---|---|---|---|---|---|---|
| 214183_s_at | X91817 | transketolase-like 1 | Y | Y | N | 131.98 |
| 214023_x_at | AL533838 | “tubulin | Y | N | | 15.2 |
| 227182_at | AW966474 | sushi domain containing 3 | Y | N | | 13.09 |
| 220779_at | NM_016233 | “peptidyl arginine deiminase | N | N | | 10.88 |
| 231729_s_at | NM_004058 | calcyphosine | N | N | | 10.82 |
| 204802_at | NM_004165 | Ras-related associated with dia [symbol] | Y | N | | 10.26 |
| 209742_s_at | AF020768 | “myosin | N | N | | 9.61 |
| 219554_at | NM_016321 | “Rhesus blood group | Y | N | | 9.36 |
| 201387_s_at | NM_004181 | ubiquitin carboxyl-terminal este [symbol] | Y | N | | 8.92 |
| 204803_s_at | NM_004165 | Ras-related associated with dia [symbol] | Y | N | | 8.64 |
| 224997_x_at | AL575306 | “H19 | Y | Y | N | 7.67 |
| 1563357_at | AL049245 | “Serine (or cysteine) proteinase | Y | Y | Y | 7.66 |
| 214368_at | AI688812 | RAS guanyl releasing protein 2 | Y | N | | 7.03 |
| 223734_at | AF329088 | ovary-specific acidic protein | Y | N | | 6.93 |
| 239430_at | AA195677 | insulin growth factor-like family | N | N | | 6.75 |
| 222746_s_at | AJ276691 | B-box and SPRY domain conta [symbol] | Y | N | | 6.57 |
| 221909_at | AW299700 | hypothetical protein FLJ14627 | N | N | | 6.49 |
| 209723_at | BC002538 | “serine (or cysteine) proteinase | Y | N | | 6.28 |
| 227711_at | BG150433 | hypothetical protein FLJ32942 | N | N | | 6.28 |
| 210130_s_at | AF096304 | transmembrane 7 superfamily n [symbol] | Y | N | | 6.19 |
| 241835_at | AI733297 | LOC440570 | N | N | | 5.98 |
| 219429_at | NM_024306 | fatty acid 2-hydroxylase | Y | N | | 5.62 |
| 230675_at | BE671925 | LOC441546 | N | N | | 6.6 |
| 211573_x_at | M98478 | “transglutaminase 2 (C polypep [symbol] | Y | Y | Y | 5.55 |
| 220921_at | NM_013453 | “SPANX family | N | N | | 5.46 |
| 227468_at | AL565745 | carnitine palmitoyltransferase 1 | Y | N | | 5.34 |
| 219045_at | NM_019034 | “ras homolog gene family | Y | N | | 5.05 |
| 202283_at | NM_002615 | “serine (or cysteine) proteinase | N | N | | 5.05 |
| 227240_at | AV703769 | neuronal guanine nucleotide ex [symbol] | N | N | | 4.33 |
| 218918_at | NM_020379 | “mannosidase | Y | N | | 4.29 |
| 1558216_at | BC043614 | hypothetical protein LOC25484 [symbol] | N | N | | 4.29 |
| 228132_at | AI240129 | “actin binding LIM protein family [symbol] | Y | N | | 4.16 |
| 227890_at | AL524643 | similar to RIKEN cDNA A23007 [symbol] | Y | N | | 4.09 |
| 215813_s_at | S36219 | prostaglandin-endoperoxide syr [symbol] | Y | N | | 3.99 |
| 238669_at | BE613133 | prostaglandin-endoperoxide syr [symbol] | Y | N | | 3.93 |
| 222812_s_at | AF239923 | “ras homolog gene family | Y | N | | 3.91 |
| 205127_at | NM_000962 | prostaglandin-endoperoxide syr [symbol] | Y | N | | 3.62 |
| 219529_at | NM_004669 | chloride intracellular channel 3 | N | N | | 3.48 |
| 1554539_a_at | BC018208 | “ras homolog gene family | Y | Y | Y | 3.45 |
| 223549_s_at | AL136880 | espin | Y | N | | 3.4 |
| 226771_at | AB032963 | “ATPase | N | N | | 3.32 |
| 224818_at | BE622952 | sortilin 1 | N | N | | 3.32 |
| 205508_at | NM_001037 | “sodium channel | Y | N | | 3.16 |
| 224378_x_at | AF276658 | microtubule-associated protein | Y | N | | 3.15 |
| 205691_at | NM_004209 | synaptogyrin 3 | Y | N | | 2.99 |
| 232164_s_at | AL137725 | epiplakin 1 | Y | N | | 2.73 |

[symbol]

indicates data missing or illegible when filed

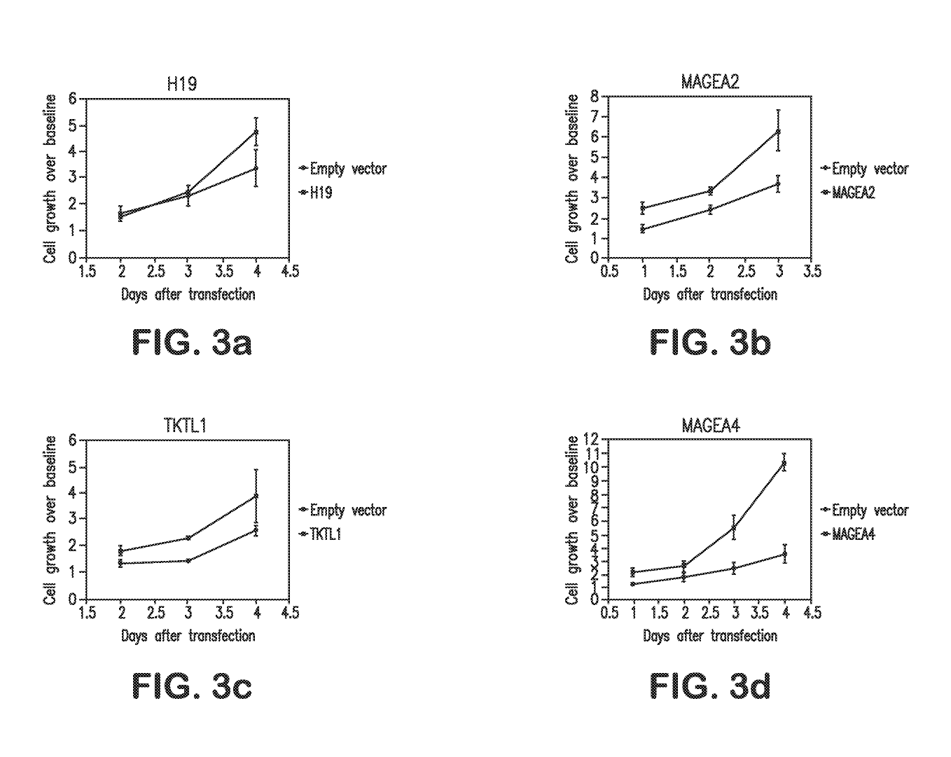

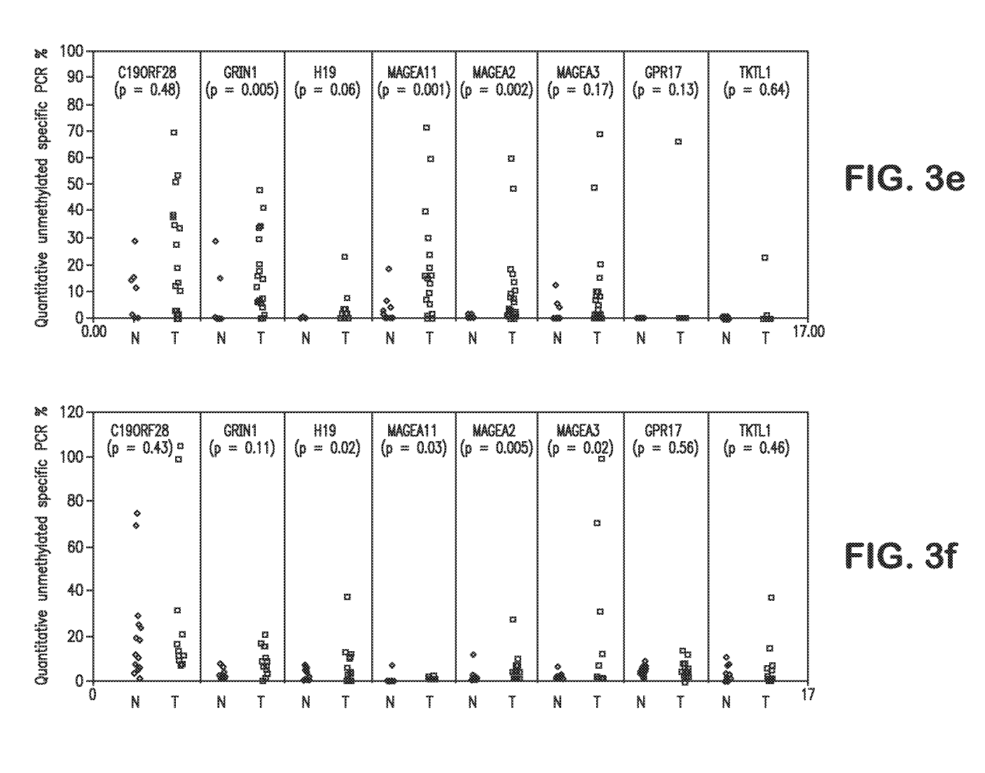

Functional validation of candidate proto-oncogenes. Transient transfections were then performed to evaluate and/or confirm growth-promoting effects of these nine targets that showed transcriptional upregulation in primary tissue with concomitant tumor-specific promoter hypomethylation. Although H19 codes for a nontranslated RNA transcript, the H19 product appears to induce growth in lung and breast cancer cell lines (Barsyte-Lovejoy et al., Cancer Res 66:5330-7, 2006) and may induce drug resistance in hepato-cellular carcinoma (Tsang and Kwok, Oncogene 26(33):4877-81, 2007). FIG. 3A shows results obtained by transient transfection of an H19 construct into OKF6-Tert-1R cells. At four days, there was a 41.4% (±15%) increase in growth over control cells transfected with empty vector. The MAGE family consists of related family members that are known to be upregulated in a variety of tumor types (Tsai et al. Lung Cancer 56(2):185-92, 2007), but have recently been implicated in inducing transcriptional reprogramming in tumor cells (Laduron et al., Nucleic Acids Res 32:4340-50, 2007). MAGEA2 induced a 72.7% (±26%) increase in growth at day three (FIG. 3B). MAGEA4 transfection induced a 203% (±17%) increase in growth (FIG. 3D). Functional growth differences were tested, but not found for C19ORF28.

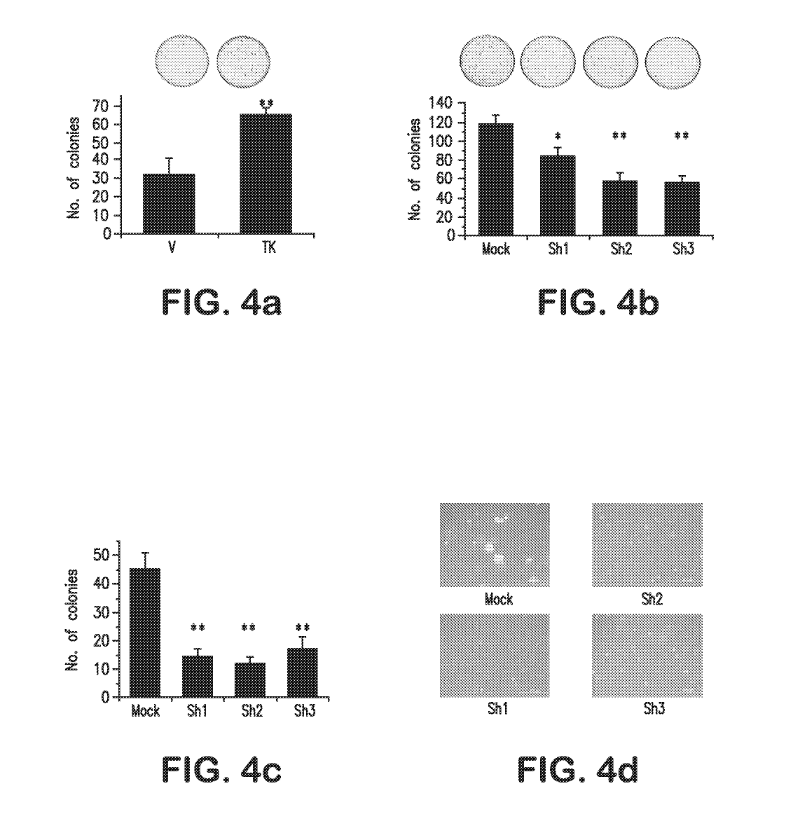

In FIG. 3C, TKTL1 induced a 50.1% (±38%) increase in growth at day four. Enhanced expression of TKTL1 has recently been implicated in the conversion of cells to aerobic, glycolytic metabolism as well as increased proliferation in colon cancer cells (Foldi et al., Oncol Rep 17:841-845, 2007; Hu et al., Anticancer Drugs 18:427-433, 2007; Krockenberger et al., Int J Gynecol Cancer 17: 101-106, 2007; Langbein et al., Br J Cancer 94:578-585, 2006; Staiger et al., Oncol Rep 16: 657-661, 2006; and Zhang et al., Cancer Lett 253(1):108-14, 2007). TKTL1 is independently associated with poor survival in laryngeal carcinoma, colon and urothelial cancers, as well as distant metastasis in ovarian carcinoma (Volker et al., Eur Arch Otorhinolaryngol 264z; 1431-6, 2007). To further confirm TKTL1 as a candidate proto-oncogene in HNSCC, adherent colony focus assays were performed in TKTL1 low-expressing HNSCC cell lines JHU-011 and JHU-028, and found significant growth increase in both cell lines (FIG. 4 A,B). shRNA constructs were then employed in a TKTL1 high-expressing cell line UM-22B in anchorage independent growth assays, and a dramatic decrease in size and number of colonies (FIG. 4 C,D) compared to mock transfected cells was noted.

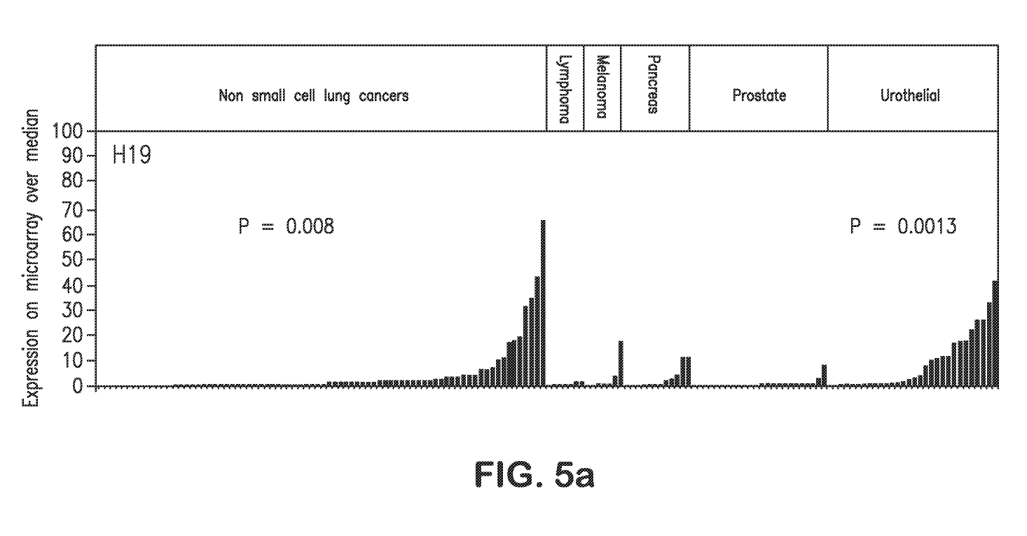

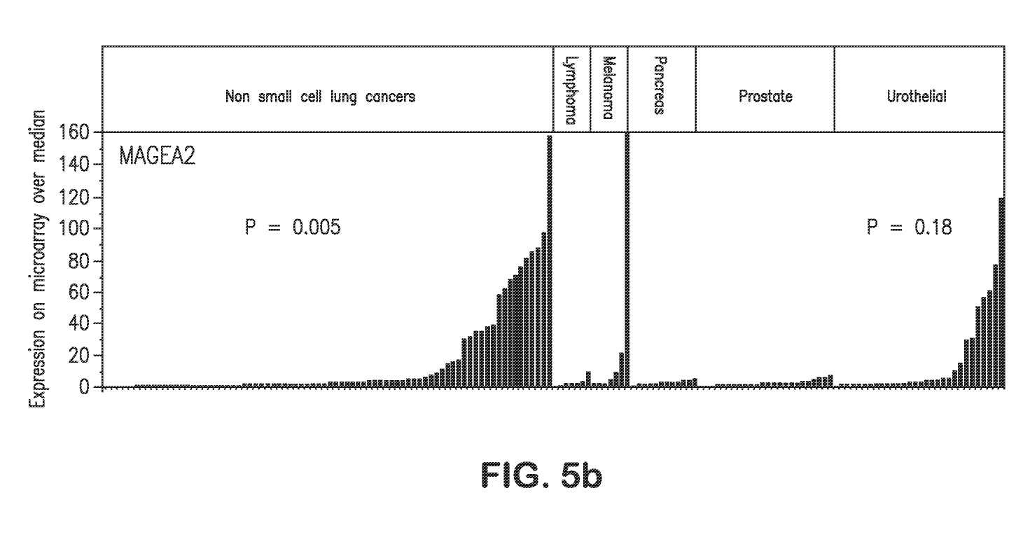

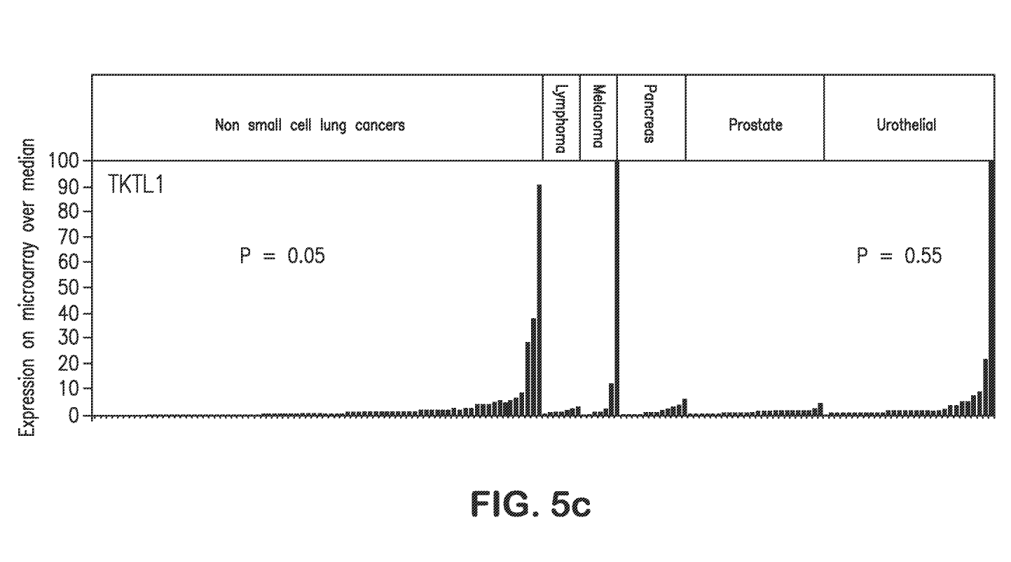

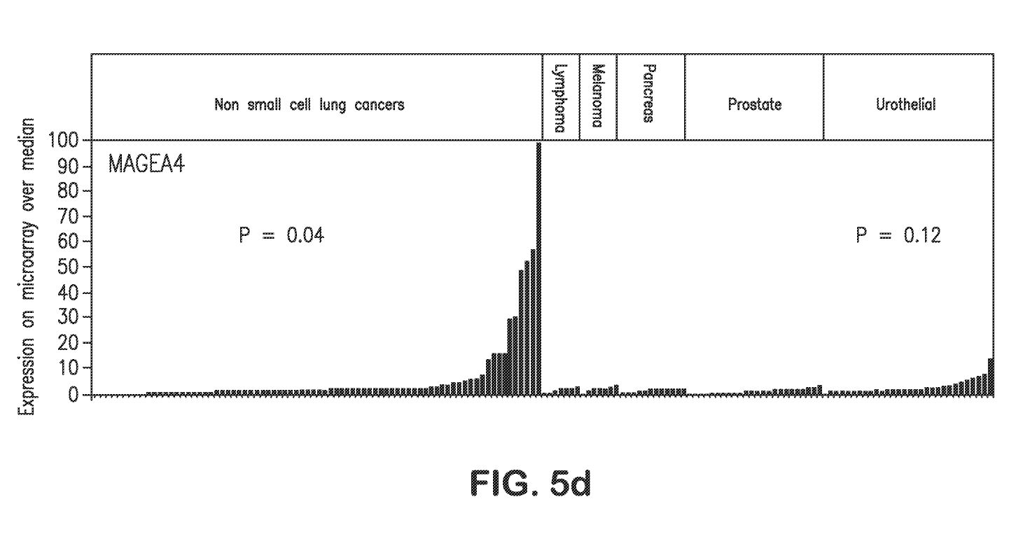

Candidate proto-oncogenes are aberrantly expressed and promoter demethylated in mutiple cancer type. To determine if candidate proto-oncogene expression was altered in a broader range of tumor types, expression data available through the expO datasets for 1041 human tumors of all histologies was analyzed. Data was first median-expression normalized by each array and subsequently by median normalization by probe set feature across the 1041 tumors from many cancer types including lung and urothelial, but not HNSCC. A subset of these tumors, non-small cell lung cancer (NSCLC), lymphoma, melanoma, pancreatic cancer, prostate cancers, and urothelial cancers, was chosen for presentation (FIG. 5A-D). H19 was significantly upregulated in NSCLC (p=0.008) and in urothelial cancer (p=0.0013), as calculated by Mann-Whitney U test comparing array-normalized expression in tumor type to all other tumors. Significantly increased expression of MAGEA2 was noted in NSCLC (p=0.005) but not in urothelial cancers (p=0.18). TKTL1 also showed overexpression in NSCLC (p=0.05), but not urothelial cancer (p=0.55), and MAGEA4 was overexpressed in NSCLC (p=0.04), but not significantly so in urothelial cancer (p=0.12). In order to confirm target-specific demethylation noted in primary tumors, a rapid, quantitative assay for specifically measuring non-methylated promoters was devised, which was termed Quantitative Unmethylation-Specific PCR (QUMSP). Twenty-five HNSCC tumors and 11 upper aerodigestive mucosal samples were assayed for promoter demethylation (FIG. 3E). Tumor-specific demethylation was found in GRIN1 (p=0.005), MAGEA11 (p=0.001), and MAGEA2 (p=0.002). A similar analysis was performed using a separate, independent cohort of 13 NSCLC samples with 14 lung samples from patients without neoplastic disease and confirmed promoter hypomethylation in target genes. Significant differences at (<0.05 in QUMSP were found in H19 (p=0.02), MAGEA11 (p=0.03), MAGEA2 (p=0.005), and MAGEA3 (p=0.02). See FIG. 3F.

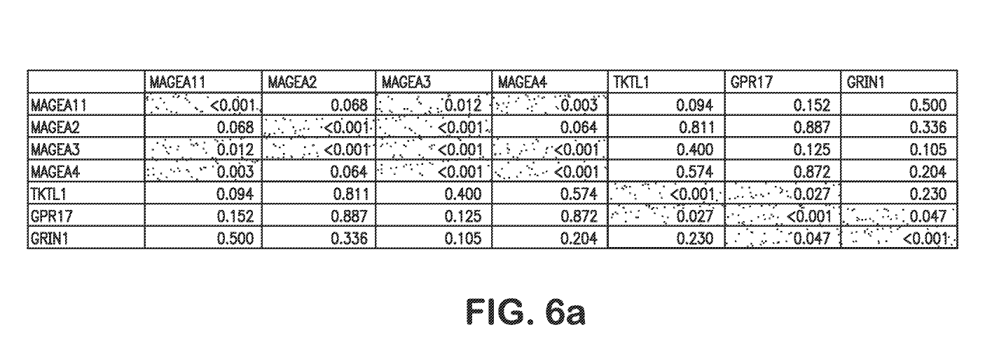

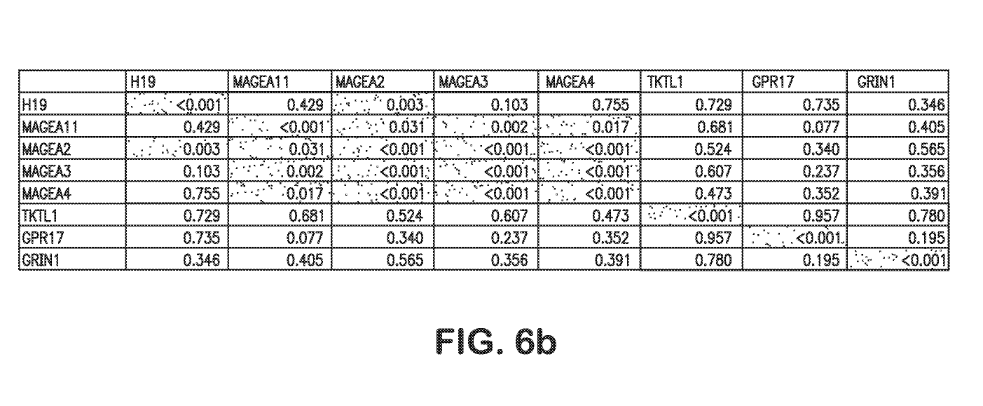

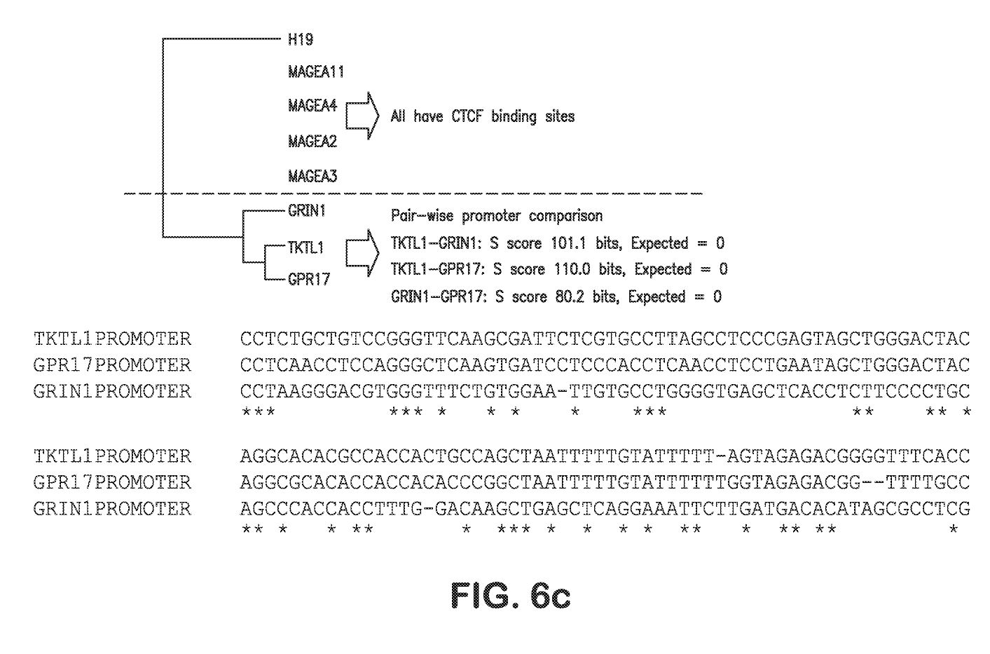

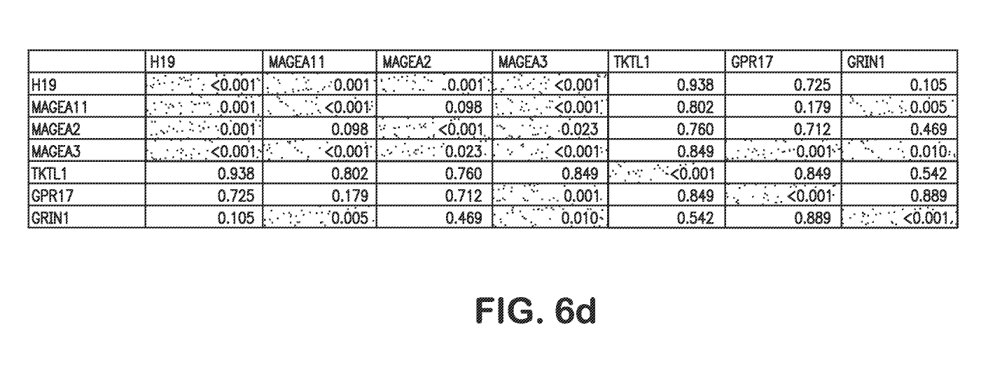

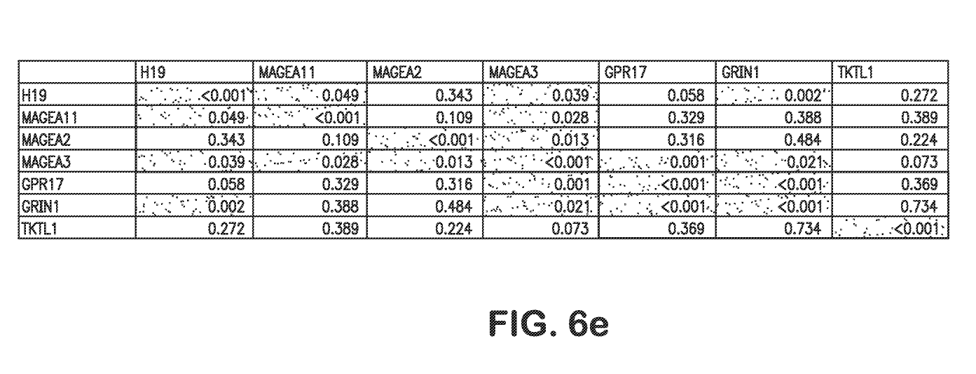

Aberrant expression of candidate proto-oncogenes occurs in a coordinated fashion in individual primary tumors. During these analyses, it was noted that transcriptional upregulation via promoter hypomethylation tended to occur synchronously in a subset of tumors. In the cohort of 49 primary HNSCC assayed via expression array analysis, a matrix of Pearson's correlation coefficients between the expression levels of each target was constructed (FIG. 6A). For the nine target genes, significant clustering of increased expression was noted within the MAGEA family of genes. H19 was not included because of its absence on the U133A platform. A separate cluster of associated overexpression was noted for TKTL1, GRIN1, and GPR17. From NSCLC expression data derived from the expO datasets we created similar matrices to examine correlations between individual genes. It was noted that MAGEA family expression and H19 expression showed highly significant correlations in individual NSCLC (see FIG. 6B). In contrast, there were no target-target correlations for NSCLC expression of the other cluster (TKTL1, GRIN1, and GPR17) that exhibited coordinated expression in HNSCC.

Expression patterns correlate with promoter homology for promoter demethylated target genes. The question as to whether promoter homology was associated with the linked expression of the two proto-oncogene clusters was then addressed. The European Bioinformatics Institutes' ClustalW tool (FIG. 6C) for phylogram analysis was used after multiple sequence alignment of the respective promoters. To confirm homology quantitatively, EMBL-EBI's PromoterWise comparison tool was used, which found significant pair-wise areas of promoter homology in GPR17, GRIN1, and TKTL1. As expected from earlier studies, the MAGE-A family clustered together, as the MAGE-A family members and H19 are known to have consensus-binding sites for methylation-sensitive binding factors CTCF and CTCFL/BORIS. In addition, this second group of GRIN1, GPR17, and TKTL1 clustered together by sequence homology.

Finally, the question as to whether the degree of promoter hypomethylation was correlated in individual tumors was addressed. For both primary HNSCC (FIG. 6D) and NSCLC (FIG. 6E), multiple significant correlations between methylation status were found between targets, but methylation status did not cluster in groups defined by the MAGE-A family/H19 expression cluster or by the TKTL1, GRIN1, GPR17 cluster. Rather, there were significant correlations between all identified candidate proto-oncogenes. Hypomethylation, therefore, appeared to occur in a related fashion in individual tumors for all target genes, but the concurrent expression of genes within the two clusters was associated with promoter homology rather than methylation status. This implied that specific transcriptional factors may be involved in the regulation of epigenetic unmasking and/or transcriptional activation based on promoter homology among these candidate proto-oncogenes.

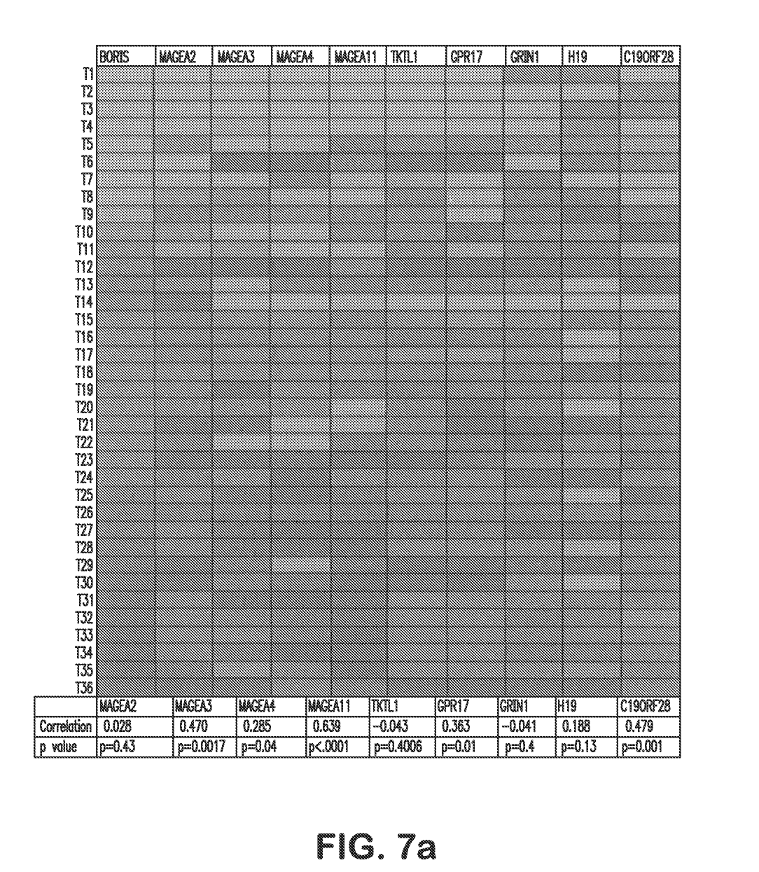

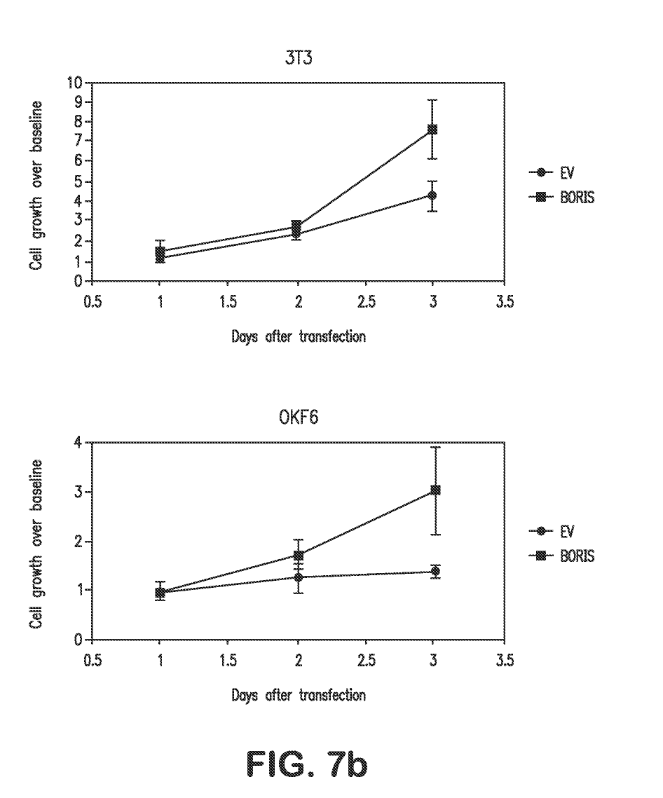

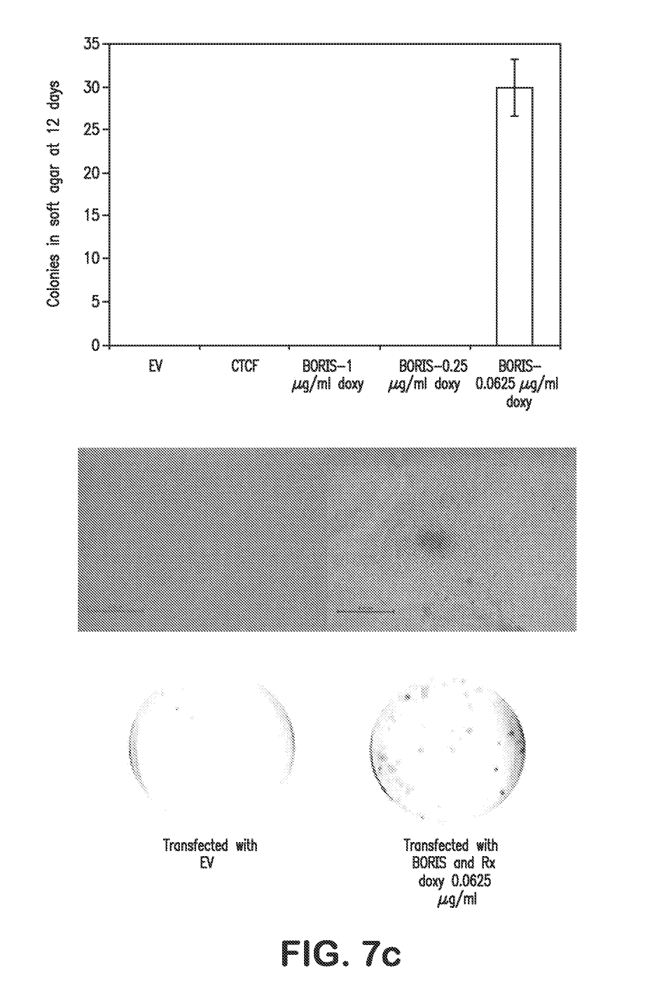

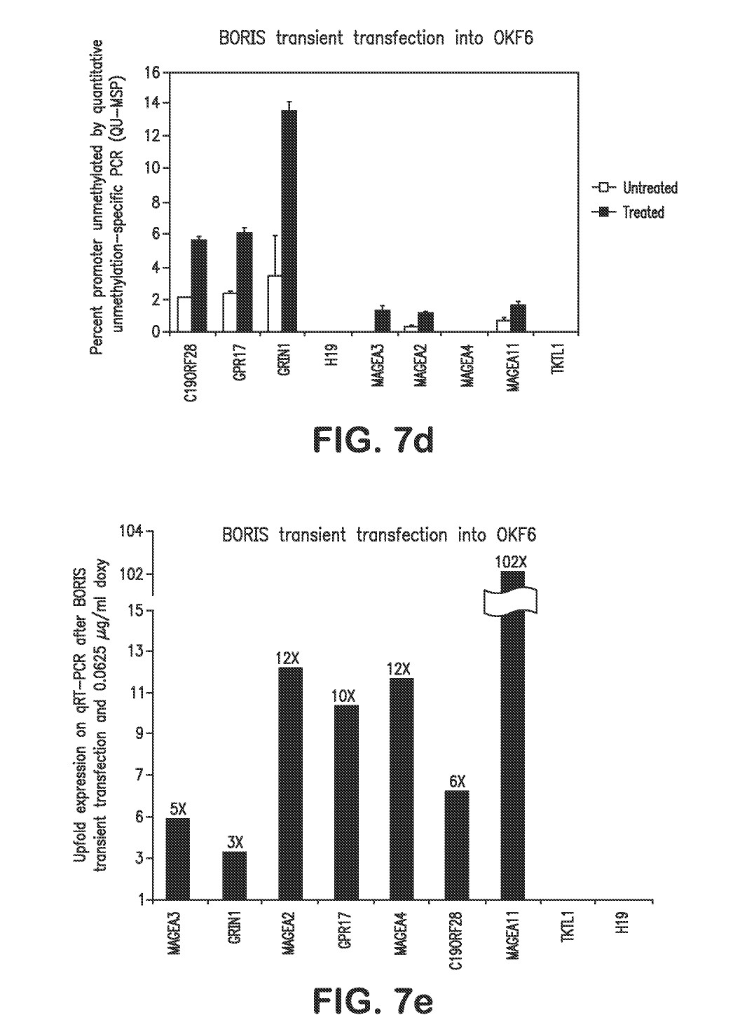

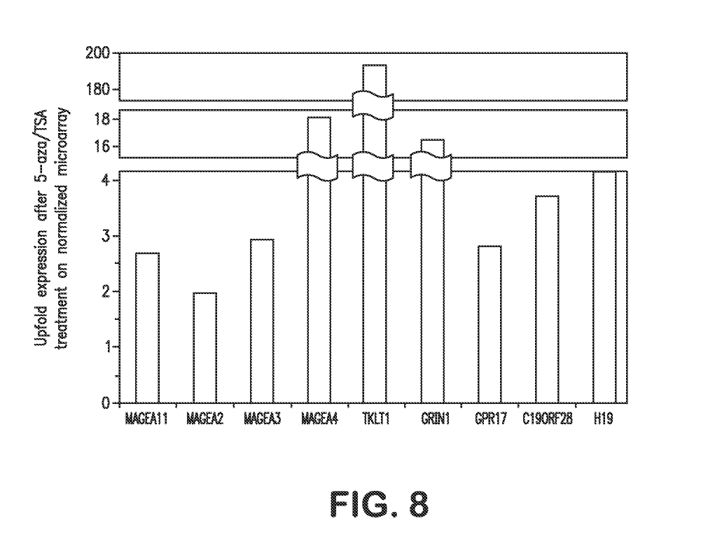

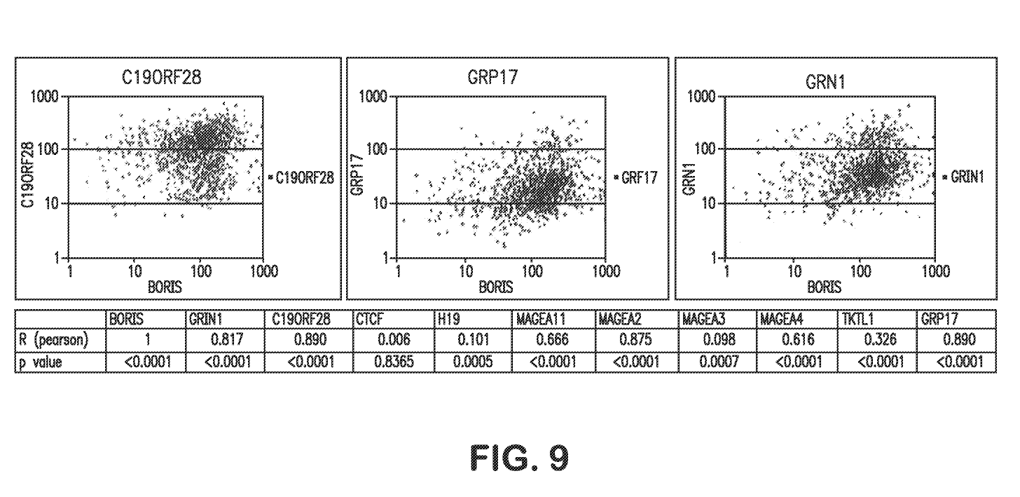

BORIS expression is associated with proto-oncogene activation in primary tumors, induces promoter demethylation, candidate proto-oncogene expression, and cell transformation. The obvious presence of several MAGE genes among the identified targets prompted the study of upstream regulatory pathways of known cancer-testis antigens. BORIS and CTCF are a unique cognate pair of transcriptional factors involved in epigenetic regulation that share an identical DNA-binding domain. BORIS is transcriptionally silenced in most normal tissues, but expressed in normal embryonic, germ cell, and cancer tissues. Thus, it was determined if expression of BORIS correlated with candidate proto-oncogene expression in a separate cohort of 36 primary HNSCC. FIG. 7A presents a heat map constructed from median normalized, qRT-PCR expression data of the proto-oncogenes, sorted by BORIS expression. In these 36 cancers, BORIS overexpression was significantly correlated to overexpression of 6/9 proto-oncogenes including: MAGEA3 (p=0.0017), MAGEA4 (p=0.04), MAGEA11 (p<0.001), GPR17 (p=0.01), and C19ORF28 (p=0.001). To further examine the correlation of BORIS expression with the target genes in solid cancers, the expO dataset data for 1041 human tumors of a wide variety of tissue sources and histologies was analyzed. Significant positive correlation of BORIS expression with expression of each of the nine proto-oncogenes was noted: GRIN1 (p<0.001), C19ORF28 (p<0.001), H19 (p<0.001), MAGEA11 (p<0.001), MAGEA2 (p<0.001), MAGEA3 (p=0.003), MAGE4 (p<0.001), TKTL1 (p<0.001), GPR17 (p<0.001), (Suppl. FIG. 2). Although BORIS transcripts are usually undetectable in normal cells, it was determined that 59% of all tumors have a BORIS level that exceeds the median expression of all genes, and 90% of tumors have a BORIS expression level >25% of median expression value for all genes, indicating that aberrant BORIS expression is a common event in human cancer.

To explore the functional and epigenetic effects of BORIS, tetracycline inducible pBIG-BORIS constructs were transiently transfected into NIH-3T3 and OKF6-Tert1R cell lines in the presence of doxycycline, resulting in increased adherent cell growth in wild type, BORIS non-expressing NIH3T3, and OKF6-Tert1R cell lines. 3T3 cells had a 77%±34% growth increase at day three. OKF6 cell lines had a 161%±78% growth increase at day three (FIG. 7B). Importantly, these effects were seen when levels of BORIS expression was regulated to be similar to the levels found in primary tumors. This effect was not seen with increased concentrations of doxycycline that induced super-“physiologic” levels of BORIS transcripts. An analysis of transcripts showed that expression of seven of nine target genes was significantly increased in OKF6-Tert1R cell expressing BORIS (FIG. 7E). To test if BORIS expressed at “physiologic” levels might contribute to transformation, NIH3T3 cells were studied for anchorage independent growth. After 12 days, significant numbers of colonies (30+/−3) were observed in tests of BORIS-expressing cells but not in cells transfected with a control plasmid (FIG. 7C).

Finally, to test the possibility that BORIS may be associated with epigenetic alterations as well as transcriptional upregulation of the identified target genes, methylation status of our candidate proto-oncogenes was quantitatively assayed after BORIS transfection and it was noted that six out of nine targets (C19 ORF28, GPR17, GRIN1, MAGEA2, MAGEA3, and MAGE11) showed a greater than 100% increase in demethylated promoter as early as 48 hours after induction of BORIS (FIG. 7D).

### Example 2

### Integrative Discovery of Epigenetically Derepressed Cancer/Testis Antigens in NSCLC

In this study, an integrative epigenetic screening approach was used to specifically identify coordinately expressed genes in human non-small cell lung cancer (NSCLC) whose transcription is driven by promoter demethylation. Our screen found 290 significant genes from the over 47,000 transcripts incorporated in the Affymetrix Human Genome U133 Plus 2.0 expression array. Of the top 55 candidates, 10 showed both differential overexpression and promoter region hypomethylation in NSCLC. Surprisingly, 6 of the 10 genes discovered by this approach were CTAs. Using a separate cohort of primary tumor and normal tissue, NSCLC promoter hypomethylation and increased expression by quantitative RT-PCR was validated for all 10 genes. Significant, coordinated coexpression of multiple target genes, as well as coordinated promoter demethylation, in a large set of individual tumors was also noted. These data suggested that epigenetic alterations are highly associated with coordinated CTA expression in NSCLC, and have significant implications for discovery of novel CTAs and CT antigen directed immunotherapy.

Histopathology. All samples were analyzed by the pathology department at Johns Hopkins Hospital. Tissues were obtained via Johns Hopkins Institutional Review Board approved protocol NA_{—}00001911. Tumor and normal lung tissues from surgical specimens were frozen in liquid nitrogen immediately after surgical resection and stored at −80° C. until use. Normal samples were microdissected and DNA prepared from normal lung parenchyma. Tumor samples were confirmed to be NSCLC and subsequently microdissected to yield at least 80% tumor cells. Tissue DNA and RNA was extracted as described below.

5Aza-dC and TSA treatment of cells. Normal human lung cell lines (NHBE and SAEC, Lonza, Walkersville, Md.) were treated in triplicate with 5-aza-deoxycytidine (5Aza-dC, a cytosine analog which cannot be methylated) and trichostatin A (TSA, a histone deacetylase inhibitor). Briefly, cells were split to low density (2.5×10⁵ cells and 6×10⁵/100 mm dish for SAEC and NHBE, respectively) 24 hours before treatment. Stock solutions of 5Aza-dC (Sigma, St. Louis, Mo.) and TSA (Sigma) were dissolved in 50% acetic acid and 100% ethanol, respectively. Cells were treated with 5 uM 5Aza-deoxycytidine for 72 hours and 300 nmol/L TSA for last 24 hours. Baseline expression was established by mock-treated cells with the same volume of acetic acid or ethanol in triplicate.

RNA extraction and oligonucleotide microarray analysis. Total cellular RNA was isolated using TRIZOL reagent (Life Technologies, Gaithersburg, Md.) and the RNEASY RNA isolation kit (Qiagen, Valencia, Calif.) according to the manufacturer's instructions. Oligonucleotide microarray analysis was carried out using the GENECHIP U133 Plus 2.0 Affymetrix expression microarray (Affymetrix, Santa Clara, Calif.). Samples were converted to labeled, fragmented, eRNA per the Affymetrix protocol for use on the expression microarray. Signal intensity and statistical significance was established for each transcript using dChip version 2005 software to initially analyze and normalize the array data and then Significance Analysis of Microarrays (SAM). SAM output was calculated at a d-value of 1.126 yielding a false discovery rate and d-score cutoff of 5.065% and 1.885. This identified a total of 12,132 upregulated candidate genes after 5Aza-dC/TSA treatment.

Public datasets. The public databases used in this study were the University of California Santa Cruz (UCSC) Human Genome reference sequence and the annotation database from the March 2006 freeze (hg18). 40 normal lung and 111 NSCLC expression microarrays were obtained from the expO datasets (all performed on the Affymetrix U133 Plus 2.0 mRNA expression platform) available online as part of the Gene Expression Omnibus (GEO/NCBI). The microarrays from normal tissue and tumor were first normalized for COPA analysis using dChip version 2005.

Cancer outlier profile analysis (COPA). COPA was applied to a cohort of 151 tissues (111 tumors, 40 normals), with each gene expression data set containing 54,613 probe sets from the Affymetrix U133 Plus 2.0 mRNA expression platform. Briefly, gene expression values were median centered, setting each gene's median expression value to zero. The median absolute deviation (MAD) was calculated and scaled to 1 by dividing each gene expression value by its MAD. Of note, median and MAD were used for transformation as opposed to mean and standard deviation so that outlier expression values do not unduly influence the distribution estimates, and are thus preserved post-normalization. Finally, the 75th, 90th, and 95th percentiles of the transformed expression values were calculated for each gene and then genes were rank-ordered by their percentile scores, providing a prioritized list of outlier profiles. For the purposes of the rank-list, the 90th percentile for tumors was chosen based on sample-size analysis (111 tumors, 40 normals). Normal tissue that had a 95th percentile >2 was eliminated from the rank list. A total of 35,764 transcripts met the above criteria and were ranked. For details of the method refer to Tomlins et al. (Science 310:644-8, 2005).

Integrative epigenetics. Target genes from the Affymetrix U133 Plus 2.0 mRNA expression platform by COPA upregulation were ranked at the 90^{th} percentile (from 111 tumors and 40 normal tissues). The U133 Plus 2.0 mRNA expression platform (Affymetrix, Santa Clara Calif.) has approximately 55,000 probe sets. A second rank list was produced by ranking genes in descending order of their d-score as computed by SAM following 5-aza/TSA treatment of normal lung cell lines (NHBE and SAEC). A third rank list was computed using 111 NSCLC and an additional expO dataset with 79 additional NSCLC primary tumor tissues also run on the Affymetrix Human Genome U133 Plus 2.0 mRNA expression platform. In these 190 primary NSCLC samples, BORIS expression patterns within each tumor was correlated with expression of all transcripts incorporated in the U133 Plus 2.0 array by calculating a correlation coefficient using Excel. All genes were then ranked based on the strength of the correlation between their expression and that of BORIS expression across all 190 samples. These three sources of information (gene set demonstrating upregulation with 5-aza, COPA score, and BORIS correlation) were combined by using a rank product (x*y*z). These three rankings were combined to rank all targets and permutation of the data was used to establish significance with a threshold of α=0.005, yielding 290 significant genes. Genomic sequences were obtained for 122 of these genes using the UCSC genome browser, and the presence of CpG islands in the promoters or first intron of these genes was determined by MethPrimer which relies on GC content of >50%, >100 bp, >0.6 observed to expected CG's.

DNA extraction. Samples were centrifuged and digested in a solution of detergent (sodium dodecylsulfate) and proteinase K, for removal of proteins bound to the DNA. DNA was purified by phenol-chloroform extraction and ethanol precipitation. The DNA was subsequently resuspended in 500 μL of LoTE (EDTA 2.5 mmol/L and Tris-HCl 10 mmol/L) and stored at −80° C. until use.

Bisulfite treatment and sequencing. 2 ug of DNA from 28 NSCLCs and 11 normal lung tissues were subjected to bisulfite treatment using the EpiTect® Bisulfite Kit (Qiagen, Valencia, Calif.) according to the manufacturer's instructions. This bisulfite-modified DNA was then stored at −80° C. Subsequently, bisulfite-treated DNA was amplified using primers designed by MethPrimer to span areas of CpG islands in the promoter or first intron (Li et al., Bioinformatics 18:1427-31, 2002). Primer sequences were designed to not contain CG dinucleotides (see Table 5 below for primer sequences). The PCR products were gel-purified using the QIAQUICK gel extraction kit (Qiagen, Valencia, Calif.), according to the manufacturer's instructions. Each amplified DNA sample was applied with nested primers to the Applied Biosystems 3700 DNA analyzer using BD terminator dye (Applied Biosystems, Foster City, Calif.).

Quantitative RT-PCR. Total RNA extracted as described above and the concentration for each sample was measured. 1 ug of RNA was then used for cDNA synthesis performed using oligo-dt with the SUPERSCRIPT First-Strand Synthesis kit (Invitrogen, Carlsbad, Calif.). The final cDNA products were used as the templates for subsequent RT-PCR with primers designed specifically for each candidate gene. 18s rRNA was examined to ensure accurate relative quantitation in quantitative RT-PCR. Each experiment was performed in triplicate using the TAQMAN 7900 (ABI) real-time PCR machine and the QUANTIFAST SYBR Green PCR Kit (Qiagen, Valencia, Calif.) according to the manufacturer's instructions.

Quantitative unmethylation-specific PCR (QUMSP). To selectively amplify demethylated promoter regions in genes of interest, primers were designed using data from bisulfite sequencing of primary tumors which are complimentary only to bisulfite-converted sequences known to be demethylated in tumors. Primer combinations were validated using in vitro methylated and demethylated controls. These experiments were performed in triplicate using the QUANTIFAST SYBR green PCR kit and the TAQMAN 7900 (ABI) real-time PCR machine with standard curves and normalization to beta-actin primers that do not contain CpG's in the sequence.

TABLE 5
QUMSP Primers

| | | SEQ |
|---|---|---|
| Primer | | ID |
| Name | Sequence | NO: |
| quMAGEA3F | TTGTGAGATTTTTGTTTTGAGTAATG | 39 |
| quMAGEA3R | CAACCTAAAAATCTTCCCCTACAA | 40 |
| quMAGEA12F | TTTTGTTTTGAGTAATGGTTTGATG | 41 |
| quMAGEA12R | CCCTCTATCTAAAATAAAACCCACC | 42 |
| quMAGEA4F | ATTTATATTTTTATTTAGGTAGGATTTTTG | 43 |
| quMAGEA4R | TCTCAAACTACAAATCAAACACA | 44 |
| quMAGEA1F | TTTGGTTTTTGTTAGGAAATATTTG | 45 |
| quMAGEA1R | ACAAAACCTAAATCAAATTCCTTCA | 46 |
| quSBSN_F | TAAGATGTGATTGGTTTAGATGTTGA | 47 |
| quSBSN_R | CCTACAACCTACCATACCCACT | 48 |
| quTKTL1F | TAAATATTTTAGATTTTTTGATTTGTTTGT | 49 |
| quTKTL1R | TAATATCAATACAACTCCCTCCACA | 50 |
| quMAGEA5F | TGGTTTTGTTTTTGTTTTTAATTGA | 51 |
| quMAGEA5R | AAATAACCCAAACTAAAAAATCCAC | 52 |
| quZNF711F | TTTTTATTTTGTAGTAAGGGTAGTGTGAT | 53 |
| quZNF711R | CCCTTAAAATCCTCACATTCAAA | 54 |
| quNYESO1F | GGTATTGTGGTTATTTTTTGGTTTG | 55 |
| quNYESO1R | CACACAAAAACCCTACTTCCAAC | 56 |
| quG6PD_F | GTTTTGGAGAGAAGTTTGAGTTTGT | 57 |
| quG6PD_R | TCCAAAAAACAAAAAAACATATAAACA | 58 |

Statistical analysis. Similarities in the methylation patterns between genes were identified by performing an analysis of correlations between QUMSP readings on the genes across all samples. Spearman's correlation permutation testing was used with 1000 permutations of the samples to establish significance, with α=0.05. For the expression data, the normalized data was log-transformed and correlation analysis was performed across all samples between each of the genes in the study. Significance was determined by assuming a normal distribution in the log-transformed expression levels and applying Student's t-distribution with an alpha of 0.05. All analyses were performed using Matlab.

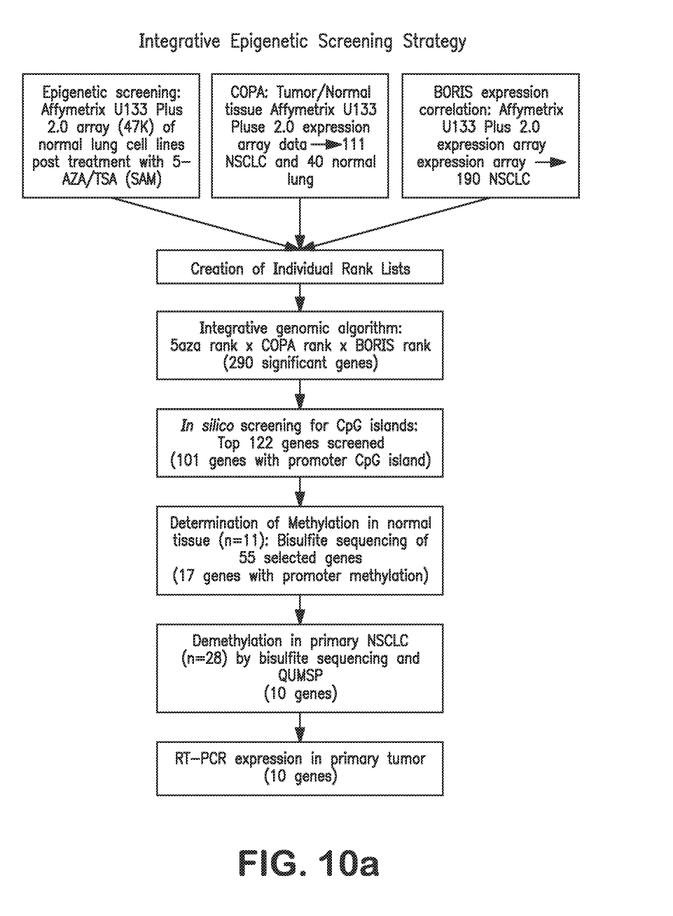

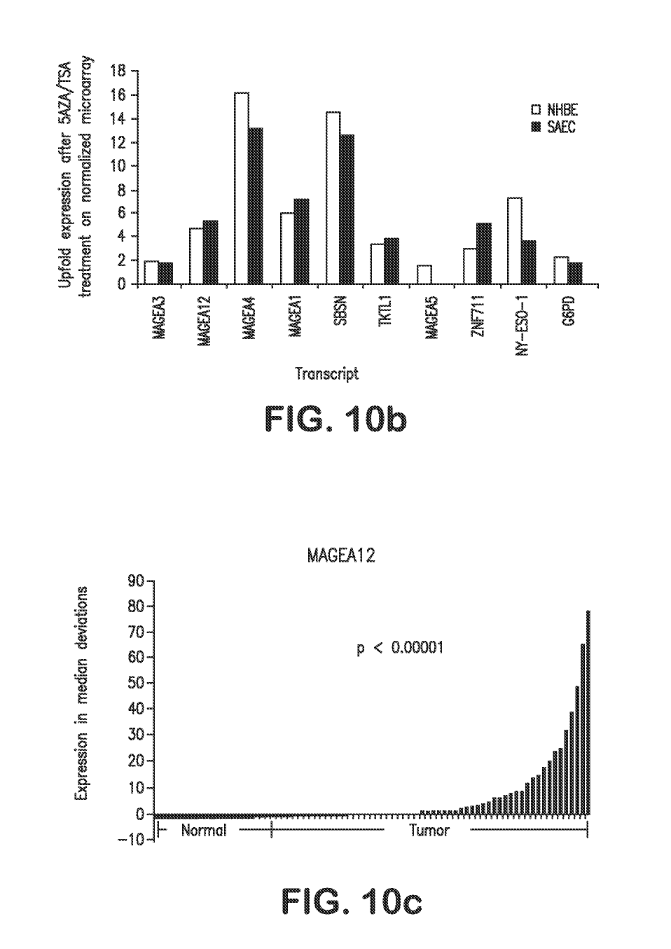

A novel integrative epigenetic approach to screen for CTAs and related epigenetically regulated genes. An integrative, high-throughput approach to screen for CTAs and other coordinately expressed genes was developed based on three key previously published factors: (1) CTAs are expressed in germline cells and many tumors, but not in normal somatic tissue, (2) CTAs have promoter CpG islands that are methylated and silenced in normal somatic tissue, but, experimentally, can be expressed by promoter demethylation and (3) the transcription factor BORIS has been shown to induce de-repression of several CTAs in NSCLC and other tumor/tissue types (FIG. 10A).

The first arm of the screening approach used herein involved the pharmacologic demethylation of 2 normal lung cell lines, Normal Human Bronchial Epithelial (NHBE) and Human Small Airway Epithelial (SAEC) cells (Lonza, Walkersville, Md.), using a 5-aza/TSA treatment protocol that has previously been successful in defining candidate tumor suppressor genes by demethylating tumor cell lines. With the understanding that CTAs are silenced by methylation in normal tissue, normal cell lines were used to identify genes that are typically repressed in normal tissues, but can be re-expressed by pharmacologic manipulation. Two normal lung cell lines, NHBE and SAEC, were treated with 5 μM 5-aza deoxycytidine for 72 hours and Trichostatin A for 24 hours prior to harvesting total RNA for expression array analysis using the Affymetrix Human Genome U133 Plus 2.0 expression platform. These results were then analyzed using dChip and Significance Analysis of Microarrays (SAM). Genes were ranked based on their SAM score(d). SAM also reported the fold change in the mean expression of the target genes in the 5-aza/TSA treatment group versus the control group (FIG. 10B).

Data from 40 normal lung and 111 NSCLC expression microarrays from expO datasets (all run on the Affymetrix Human Genome U133 Plus 2.0 mRNA expression platform) publicly available online as part of the Gene Expression Omnibus (GEO/NCBI) were concurrently analyzed. For analysis of these 151 primary tissue expression array data sets, a technique known as Cancer Outlier Profile Analysis (COPA) was used. COPA is a method to search for marked overexpression of particular genes that occur only in a subset of cases, whereas traditional analytical methods based on standard statistical measures fail to find genes with this type of expression profile. COPA was a particularly useful method for the search for CTAs and genes with similar expression profiles based on previous studies showing that CTAs are heterogeneously expressed both across a wide patient population and within individual tumor specimens. Genes with a normal tissue COPA expression scaled score >2 at the 95th percentile were eliminated from the rank list. All remaining genes were then ranked based on their COPA score at the 90^{th} percentile; statistical significance of the expression differences in the COPA diagrams were measured by Mann-Whitney U test (FIG. 10C).

For the final arm of the screening approach provided herein, the previous data set with 111 NSCLC and an additional expO dataset with 79 additional NSCLC primary tumor tissues also run on the Affymetrix Human Genome U133 Plus 2.0 mRNA expression platform was used. In these 190 primary NSCLC samples, BORIS expression patterns within each tumor were correlated with expression of all transcripts incorporated in the U133 Plus 2.0 array by calculating a correlation coefficient using Excel. All genes were then ranked based on the strength of the correlation between their expression and that of BORIS expression across all 190 samples.

Three rank lists were produced by ranking genes by SAM score(d) following 5-aza/TSA treatment in normal lung cell lines, COPA score in primary tissue, and BORIS correlation in primary tissue. These 3 rank lists were combined by using a rank product (x*y*z). Using a significance threshold (α=0.005) and subsequent random permutations of the rank-lists, 290 genes were identified that were significantly differentially upregulated based on epigenetic screening and tissue microarray expression patterns (FIG. 17).

Initially, an in silico approach utilizing MethPrimer was used to confirm the presence of CpG islands in the promoter regions of our top candidates. The top 100 of the 290 significant genes as well as 22 genes selected based on biological relevance in cancer related pathways were selected to be screened via this approach, and 101 were found to contain 1 or more promoter CpG islands.

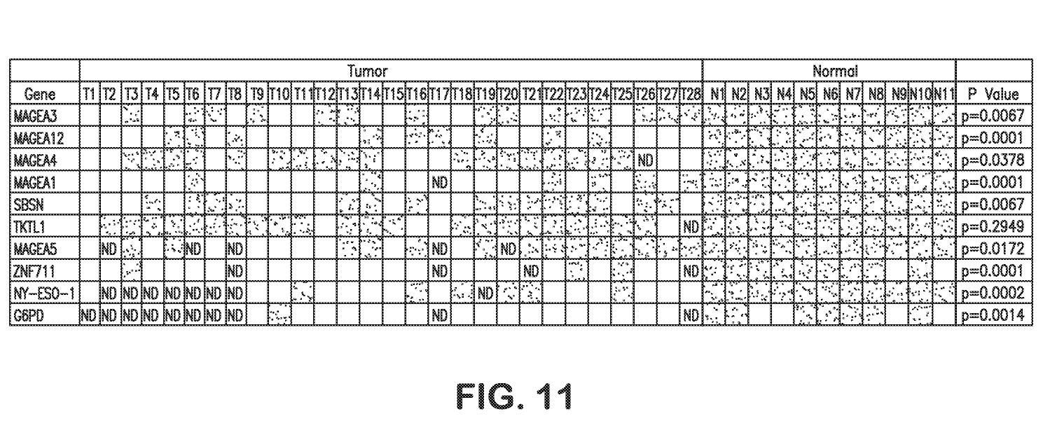

A separate cohort of 11 normal lung tissues from patients without a cancer diagnosis was then used to confirm epigenetic silencing via promoter methylation in normal lung mucosa from patients without a lung neoplasm. Bisulfite sequencing of CpG islands in the promoter regions of 55 selected gene targets with CpG islands was used to determine the methylation status. Only 17/55 promoter regions demonstrated complete methylation at all sequenced CpG sites in all or nearly all of the normal tissues (FIG. 17). These targets were subsequently bisulfite sequenced in a separate cohort of 28 primary NSCLC to search for the presence of promoter hypomethylation. Of these remaining targets, 10/17 showed promoter demethylation in some fraction of tumors including: MAGEA3 (13/28, p=0.0067), MAGEA12 (19/28, p=0.0001), MAGEA4 (9/27, p=0.0378), MAGEA1 (21/27, p=0.0001), SBSN (13/28, p=0.0067), TKTL1 (5/27, p=0.2949), MAGEA5 (9/23, p=0.0172), ZNF711 (21/24, p=0.0001), NY-ESO-1 (14/20, p=0.0002), G6PD (17/18, p=0.0014), (Fisher's exact test) (Table 6 and FIG. 11).

TABLE 6
Genes coordinately expressed and demethylated in NSCLC

| | | | | Upregulated | | | |
|---|---|---|---|---|---|---|---|
| | | | | with 5-Aza | | | |
| | | | | Average Fold | Promoter | Methylated | |
| | | Integrated | | Change | CpG | in Normal | Unmethylated |
| | | Rank | COPA | (SAM Score | Island | Lung | in NSCLC |
| Symbol | Description | Position | Score* | (d)) | Present | Tissue | Tumor Tissue |
| MAGEA3 | Melanoma | 4 | 60.6 | 1.8 (9.9) | Y | Y | Y |
| | antigen family | | | | | | |
| | A, 3 | | | | | | |
| MAGEA12 | Melanoma | 5 | 24.1 | 4.8 (7.8) | Y | Y | Y |
| | antigen family | | | | | | |
| | A, 12 | | | | | | |
| MAGEA4 | Melanoma | 6 | 101.0 | 14.9 (11.3) | Y | Y | Y |
| | antigen family | | | | | | |
| | A, 4 | | | | | | |
| MAGEA1 | Melanoma | 11 | 12.8 | 6.5 (10.5) | Y | Y | Y |
| | antigen family | | | | | | |
| | A, 1 | | | | | | |
| SBSN | Suprabasin | 27 | 6.4 | 13.5 (31.3) | Y | Y | Y |
| TKTL1 | Transketolase- | 35 | 1.8 | 3.5 (15.3) | Y | Y | Y |
| | like 1 | | | | | | |
| MAGEA5 | Melanoma | 41 | 16.9 | 1.5 (3) | Y | Y | Y |
| | antigen family | | | | | | |
| | A, 5 | | | | | | |
| ZNF711 | Zinc finger | 43 | 10.0 | 3.8 (9.3) | Y | Y | Y |
| | protein 6 | | | | | | |
| NY-ESO-1 | Cancer/testis | 72 | 117.6 | 5.9 (4) | Y | Y | Y |
| | antigen 1B | | | | | | |
| G6PD | Glucose-6- | 105 | 22.4 | 2.1 (10.6) | Y | Y | Y |
| | phosphate | | | | | | |
| | dehydrogenase | | | | | | |

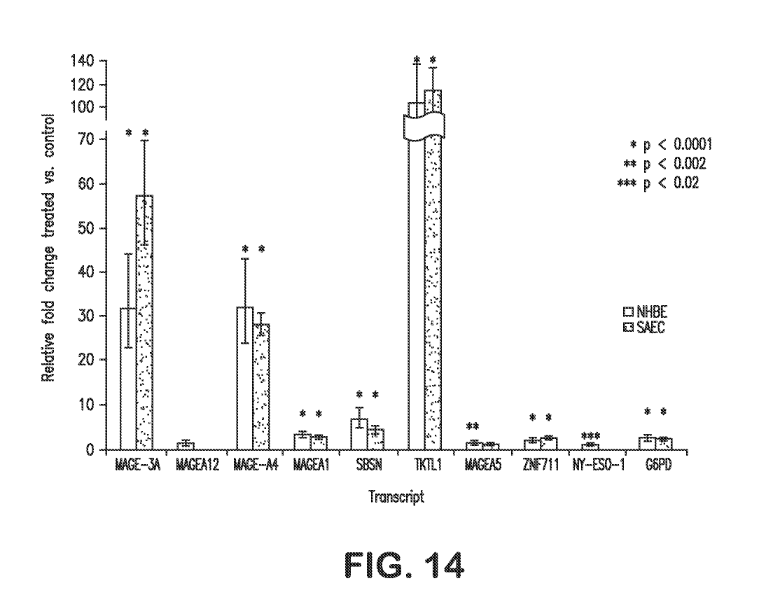

Transcriptional upregulation of target genes after 5-aza/TSA treatment in the cell line system was confirmed using quantitative RT-PCR on the 5-aza/TSA-treated normal cells compared to mock-treated cells for these 10 genes (FIG. 14). Each gene, with the exception MAGEA12, demonstrated significant upregulation by 5-aza/TSA treatment in at least one cell line supporting functional gene regulation by promoter hypomethylation.

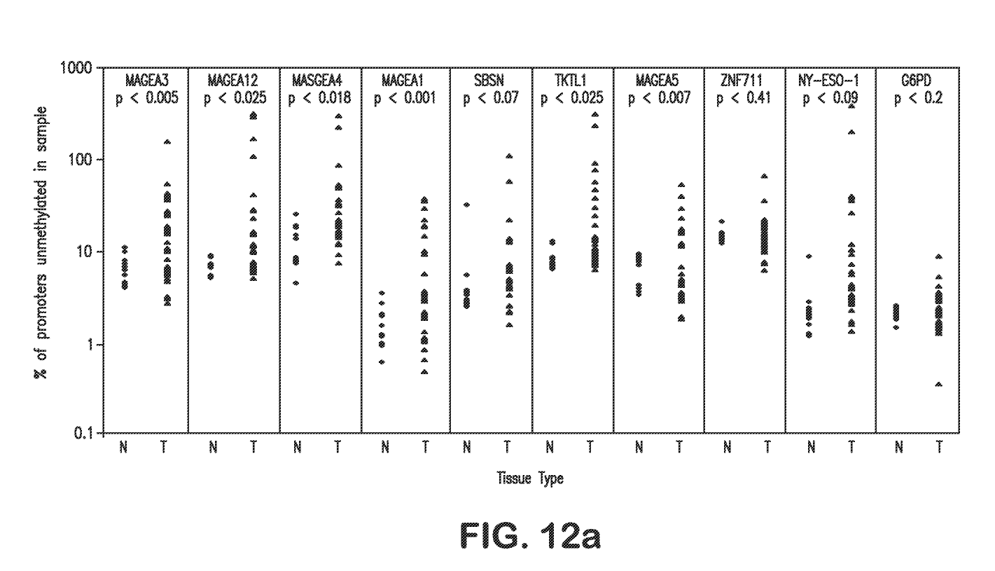

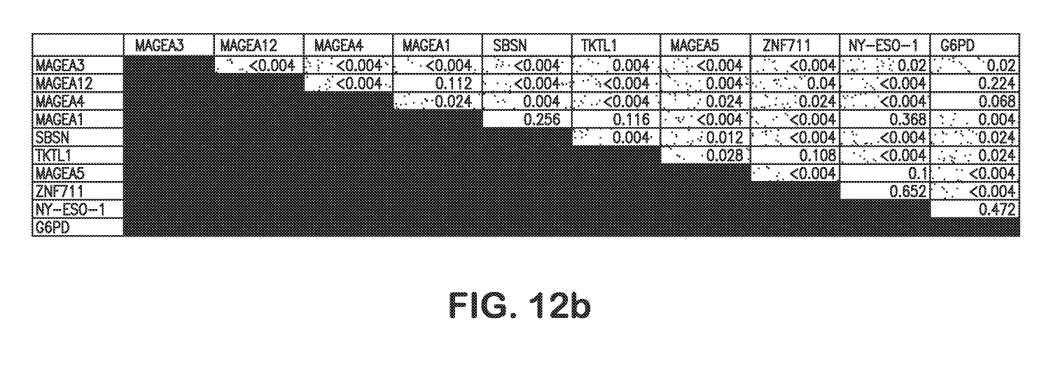

CTAs and associated genes are coordinately demethylated and expression is correlated with promoter demethylation. In order to confirm the bisulfate sequencing results in the target genes and to provide a dataset of continuous variables to express the status of promoter demethylation, a rapid, quantitative assay for specifically measuring non-methylated promoters was devised, which was termed Quantitative Unmethylation-Specific PCR (QUMSP). DNA extracted from the cohort of 28 primary NSCLC tumor samples and 11 normal lung samples from non-cancer patients was assayed (FIG. 12A). Significant tumor-specific demethylation was found in MAGEA3 (p<0.005), MAGEA12 (p<0.025), MAGEA4 (p<0.018), MAGEA1 (p<0.001), TKTL1 (p<0.025) and MAGEA5 (p<0.007). Two additional targets slightly missed significance at α<0.05, SBSN (p<0.07) and NY-ESO-1 (p<0.09) (2 tailed Student's t-test assuming unequal variance).

Given the tumor-specific demethylation pattern seen for these target genes, the question as to whether demethylation of the promoter regions of these genes occurred in a coordinated fashion within tumor samples was addressed. Spearman's correlation permutation testing was utilized to determine significant coordinated demethylation using the QUMSP results from the cohort of 28 NSCLC (FIG. 12B). The p-value matrix of the Spearman's correlation coefficient showed that for any of the target genes, demethylation tended to coordinately occur with a minimum of 6 of the other genes. Shaded cells represent significant p-values. This offered evidence that demethylation is highly associated with coordinated regulation of these CTAs and related target genes in NSCLC, and strongly suggested an epigenetic mechanism of activation.

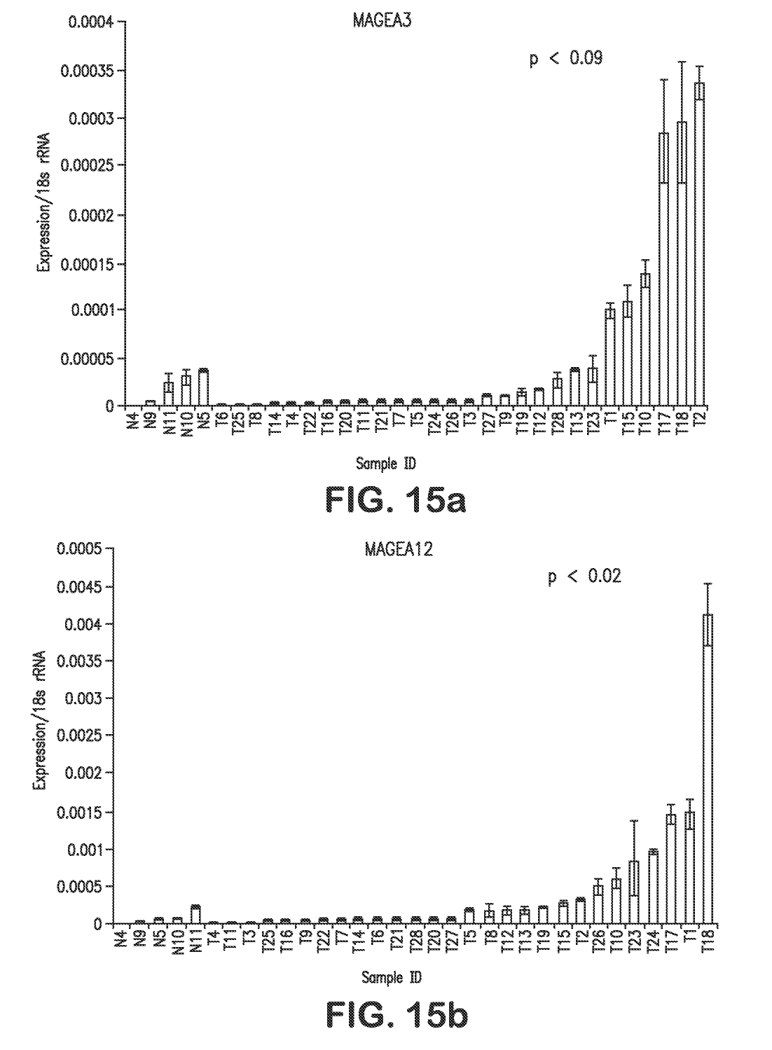

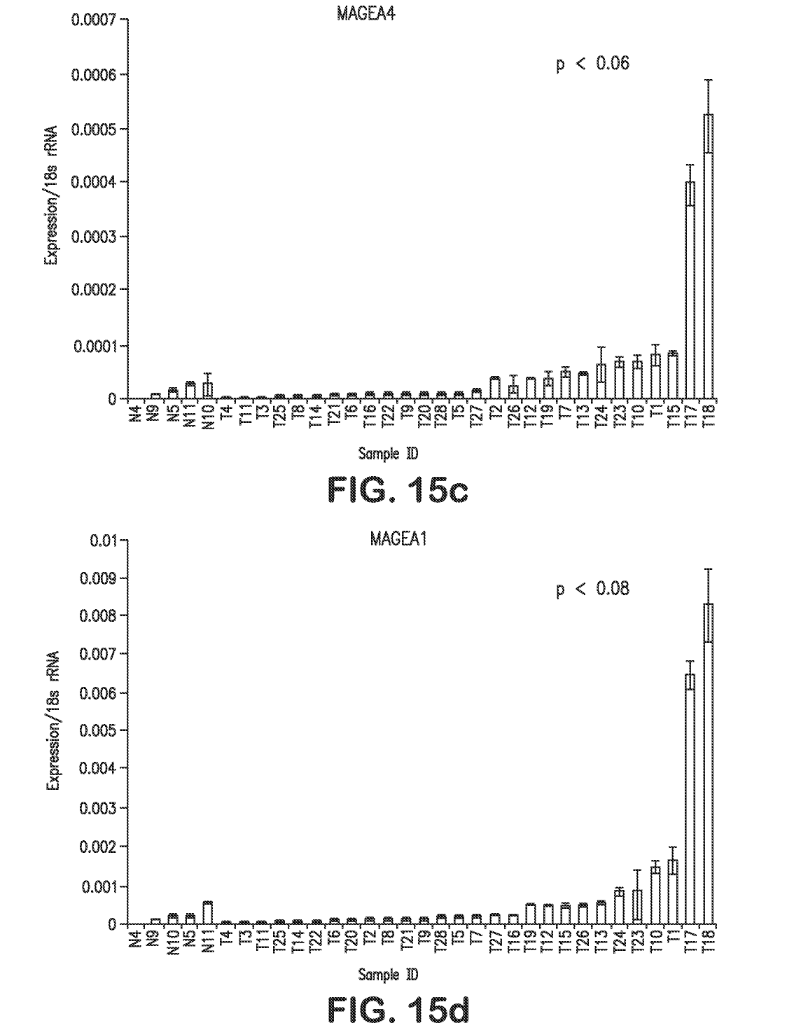

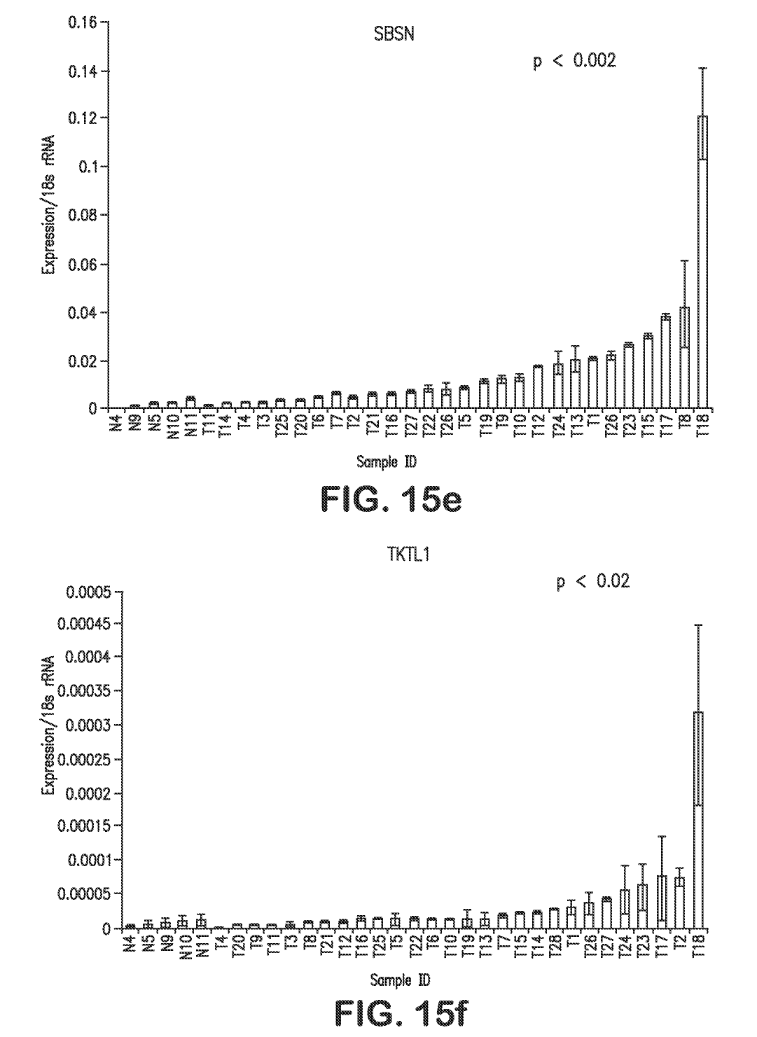

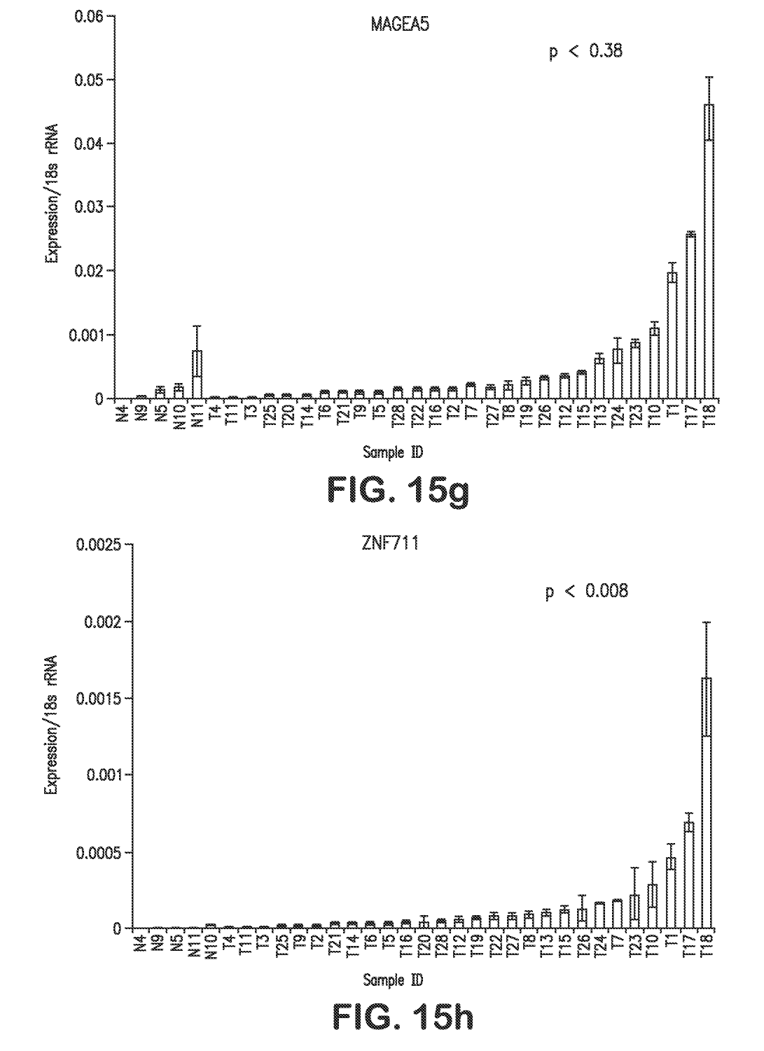

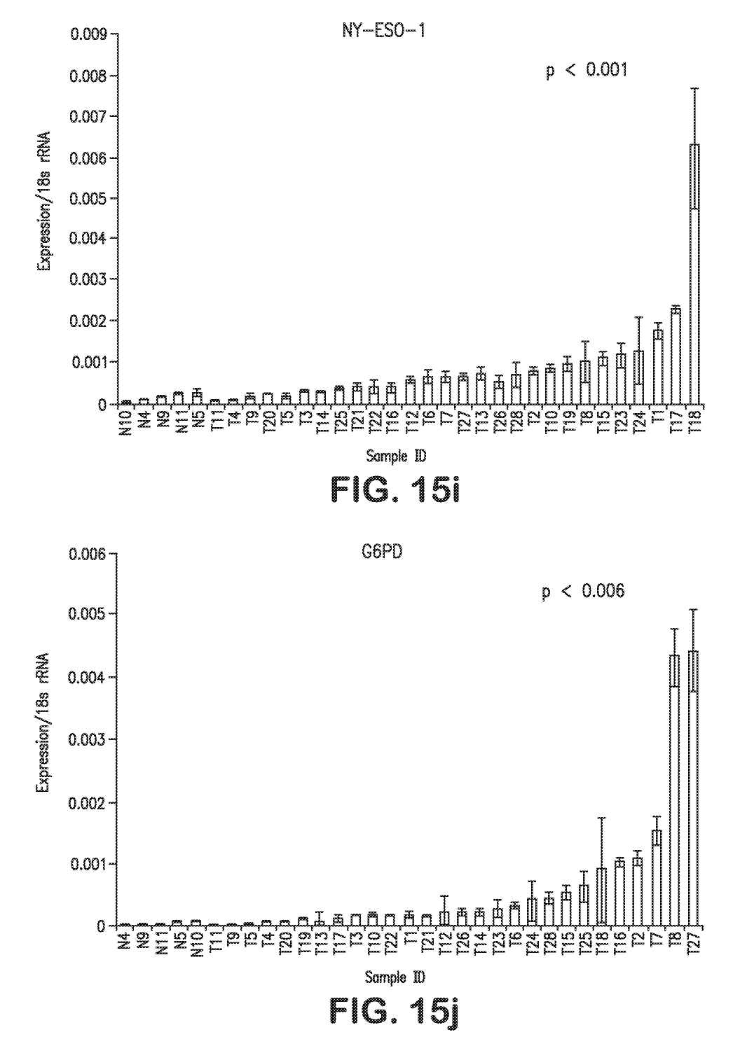

In order to confirm tumor specific expression of the target genes, quantitative RT-PCR was used to determine mRNA expression in the cohort of NSCLC and normal lung tissue (FIG. 15A-J). Six genes had significantly increased expression in tumors MAGEA12 (p<0.02), SBSN (p<0.002), TKTL1 (p<0.02), ZNF711 (p<0.008), NY-ESO-1 (p<0.001), G6PD (p<0.006). Three genes slightly missed significance at the α<0.05 level: MAGEA3 (p<0.09), MAGEA4 (p<0.06) and MAGEA1 (p<0.08) (2 tailed Student's t-test assuming unequal variance).

The question as to whether demethylation was responsible for the derepression of the CTAs and related genes in NSCLC was next addressed. Four target genes showed a significant positive correlation between mRNA expression (quantitative RT-PCR) and promoter hypomethylation (QUMSP): MAGEA12 (p=0.024), MAGEA4 (p<0.004), SBSN (p=0.004) and NY-ESO-1 (p<0.004) (FIG. 16A-D) (Spearman's correlation permutation test). TKTL1 (p=0.1), MAGEA5 (p=0.104) and MAGEA3 (p=0.2) also showed a positive correlation between demethylation and expression, but missed significance. These data suggested demethylation of promoter regions was partially responsible for the regulation of the majority of the target genes.

CTAs and associated target genes are coordinately expressed. Given the findings in the previous analyses of the cohort of primary tissue showing that these target genes were differentially expressed in tumors, their promoter regions were coordinately demethylated within tumors and expression was correlated with demethylation, the initial cohort of 111 tumors assayed using the Affymetrix Human Genome U133 Plus 2.0 mRNA expression platform was examined to determine if the target genes were coordinately expressed within tumor samples in this large sample set. FIG. 4A shows a heat map of transcript expression as measured by the U133 Plus 2.0 array for 40 normal lung samples from non-cancer patients and 111 NSCLC primary tissue samples. This analysis not only provided confirmation that expression of the target genes is limited to a subset of tumors with little or no expression in the normal tissue, but also, that these targets appear to be coordinately expressed in a subset of these tumors.

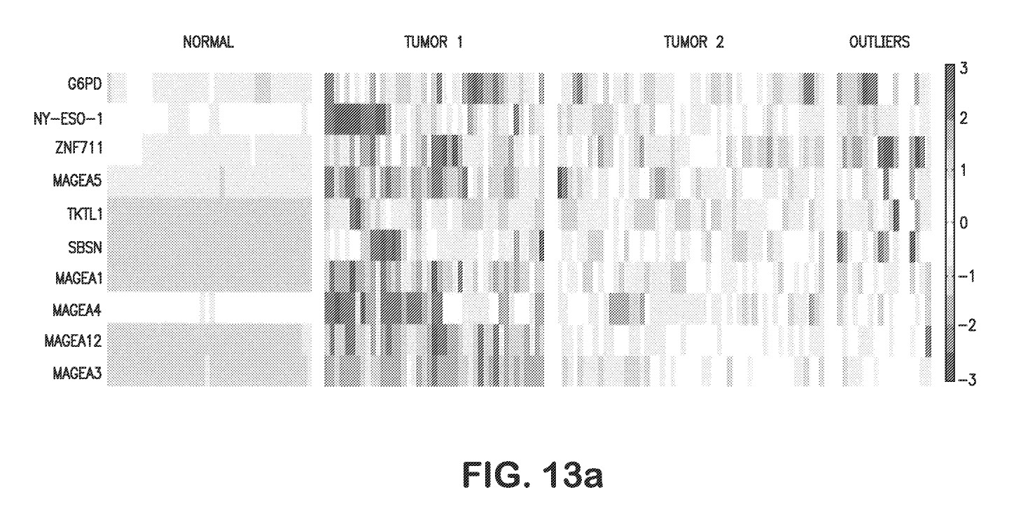

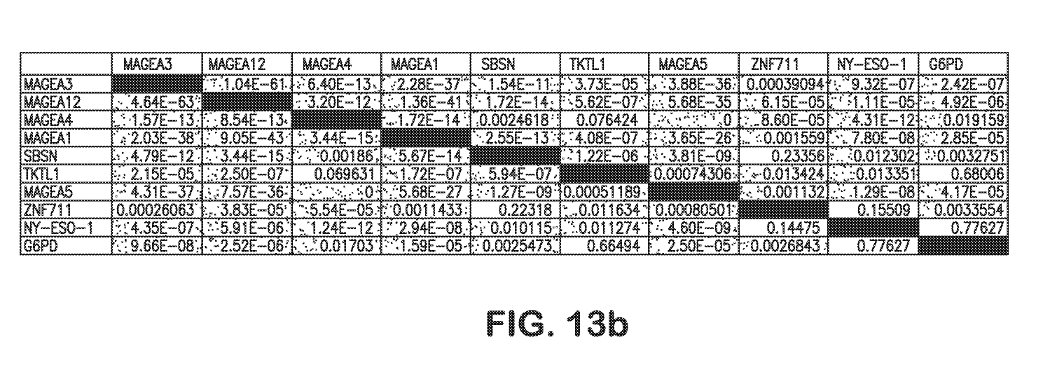

To formally test the coordinate expression of these genes, a p-value matrix derived from the Pearson's correlation coefficients calculated between the expression levels of each target was next constructed (FIG. 13B). Values to the upper right were corrected with the Benjamin Hochberg multiple test correction to decrease the false discovery rate; however, there was no change in significance after this correction (uncorrected values displayed in lower left). Shaded cells represent significant p-values. Using this pairwise comparison method, a highly significant coordinated upregulation of all 10 target genes within a subset of tumor samples was found.

Although the invention has been described with reference to the above examples, it will be understood that modifications and variations are encompassed within the spirit and scope of the invention. Accordingly, the invention is limited only by the following claims.

1. A method for identifying a cell that exhibits or is predisposed to exhibiting unregulated growth, comprising detecting hypomethylation of a gene or a regulatory region in at least one gene in the cell, wherein the at least one gene is hypomethylated as compared to a corresponding normal cell not exhibiting unregulated growth, thereby identifying the cell as exhibiting or predisposed to exhibiting unregulated growth.

2. The method of claim 1, wherein at least two genes or regulatory regions are hypomethylated.

3. The method of claim 1, wherein the regulatory region of the at least one gene comprises a BORIS binding site.

4. The method according to claim 1, wherein the regulatory region of the at least one gene comprises a promoter of a gene selected from the group consisting of TKTL1, H19, MAGEA2, MAGEA3, MAGEA4, MAGEA11, GPR17, GRIN1, C19ORF28, MAGEA12, MAGEA1, MAGEA5, NY-ESO-1, MAGEA9, MAGEA6, MAGEB2, CT45-2, SBSN, G6PD, ZNF711, CrispL, KRT86, KIPV467, KRT81, CSPG5, PP1R14A, KISS1R, KIAA1937 protein, SOX30, DEAD, and KBGP.

5. (canceled)

6. (canceled)

7. The method of claim 1, wherein the cell that exhibits or is predisposed to exhibiting unregulated growth, is from a cancer cell selected from the group consisting of head cancer, neck cancer, head and neck cancer, lung cancer, breast cancer, prostate cancer, colorectal cancer, esophageal cancer, stomach cancer, leukemia/lymphoma, uterine cancer, skin cancer, endocrine cancer, urinary cancer, pancreatic cancer, gastrointestinal cancer, ovarian cancer, cervical cancer, and adenomas.

8. The method of claim 7, wherein the cancer is head and neck cancer.

9. The method of claim 7, wherein the cancer is lung cancer.

10. The method of claim 1 wherein the hypomethylation is of a CpG dinucleotide motif in the at least one gene or regulatory region.

11. The method of claim 1 wherein the hypomethylation is of a CpG dinucleotide motif in a promoter of the regulatory region of the at least one gene.

12. The method of claim 1 wherein hypomethylation is detected by detecting increased expression of the at least one gene.

13. The method of claim 1, wherein hypomethylation is detected by detecting increased mRNA of the at least one gene.

14. (canceled)

15. (canceled)

16. The method of claim 12 wherein increased expression is detected by reverse transcription-polymerase chain reaction (RT-PCR).

17. (canceled)

18. The method of claim 12 wherein hypomethylation is detected by detecting increased protein encoded by the gene.

19. The method of claim 1 wherein hypomethylation is detected by contacting at least a portion of the gene with a methylation-sensitive restriction endonuclease, said endonuclease preferentially cleaving non-methylated recognition sites relative to methylated recognition sites, whereby cleavage of the portion of the gene indicates non-methylation of the portion of the gene provided that the gene comprises a recognition site for the methylation-sensitive restriction endonuclease.

20. The method of claim 1 wherein hypomethylation is detected by contacting at least a portion of the gene of the cell with a chemical reagent that selectively modifies a non-methylated cytosine residue relative to a methylated cytosine residue, or selectively modifies a methylated cytosine residue relative to a non-methylated cytosine residue; and detecting a product generated by the contacting step.

21. The method of claim 20 wherein the step of detecting comprises hybridization with at least one probe that hybridizes to a sequence comprising a modified non-methylated CpG dinucleotide motif but not to a sequence comprising an unmodified methylated CpG dinucleotide.

22. The method of claim 20 wherein the step of detecting comprises amplification with at least one primer that hybridizes to a sequence comprising a modified non-methylated CpG dinucleotide motif but not to a sequence comprising an unmodified methylated CpG dinucleotide motif thereby forming amplification products.

23. The method of claim 20 wherein the step of detecting comprises amplification with at least one primer that hybridizes to a sequence comprising an unmodified methylated CpG dinucleotide motif but not to a sequence comprising a modified non-methylated CpG dinucleotide motif thereby forming amplification products.

24. The method of claim 20 wherein the product is detected by a method selected from the group consisting of electrophoresis, hybridization, amplification, primer extension, sequencing, ligase chain reaction, chromatography, mass spectrometry, and combinations thereof.

25. The method of claim 20 wherein the chemical reagent is hydrazine.

26. The method of claim 25 further comprising cleaving the hydrazine-contacted at least a portion of the gene with piperidine.

27. The method of claim 20 wherein the chemical reagent comprises bisulfite ions.

28. The method of claim 27 further comprising treating the bisulfite ion-contacted at least a portion of the gene with alkali.

29. A method for diagnosing a disorder in a subject having or at risk of developing a cell proliferative disorder comprising:
contacting a nucleic acid-containing sample from cells of the subject with an agent that provides a determination of the methylation state of at least one regulatory region of a gene, wherein the at least one regulatory region is demethylated as compared to a corresponding normal cell; and
identifying hypomethylation of the regulatory region as compared to the same region of the at least one regulatory region in a subject not having the proliferative disorder, wherein hypomethylation is indicative of a subject having or at risk of developing the proliferative disorder.

30. The method of claim 29, wherein at least two regulatory regions are hypomethylated.

31. The method of claim 29, wherein the regulatory region of the at least one gene comprises a BORIS binding site.

32. The method according to claim 29, wherein the regulatory region of the at least one gene comprises a promoter of a gene selected from the group consisting of TKTL1, H19, MAGEA2, MAGEA3, MAGEA4, MAGEA11, GPR17, GRIN1, C19ORF28, MAGEA12, MAGEA1, MAGEA5, NY-ESO-1, MAGEA9, MAGEA6, MAGEB2, CT45-2, SBSN, G6PD, ZNF711, CrispL, KRT86, KIPV467, KRT81, CSPG5, PP1R14A, KISS1R, KIAA1937 protein, SOX30, DEAD, and KBGP.

33. (canceled)

34. (canceled)

35. The method of claim 29, wherein the cell proliferative disorder is selected from the group consisting of head cancer, neck cancer, head and neck cancer, lung cancer, breast cancer, prostate cancer, colorectal cancer, esophageal cancer, stomach cancer, leukemia/lymphoma, uterine cancer, skin cancer, endocrine cancer, urinary cancer, pancreatic cancer, gastrointestinal cancer, ovarian cancer, cervical cancer, and adenomas.

36. The method of claim 35, wherein the cancer is head and neck cancer.

37. The method of claim 35, wherein the cancer is lung cancer.

38. The method of claim 29 wherein the cells are from a sample selected from the group consisting of a tissue sample, a frozen tissue sample, a biopsy specimen, a surgical specimen, a cytological specimen, whole blood, bone marrow, cerebral spinal fluid, peritoneal fluid, pleural fluid, lymph fluid, serum, mucus, plasma, urine, chyle, stool, ejaculate, sputum, nipple aspirate and saliva.

39. The method of claim 29 wherein the hypomethylation is of a CpG dinucleotide motif in the at least one gene or regulatory region.

40. The method of claim 29 hypomethylation is of a CpG dinucleotide motif in a promoter of the regulatory region of the at least one gene.

41. The method of claim 29 wherein hypomethylation is detected by detecting increased expression of the at least one gene.

42. The method of claim 29 wherein hypomethylation is detected by detecting increased mRNA of the at least one gene.

43. The method of claim 41 wherein increased expression is detected by reverse transcription-polymerase chain reaction (RT-PCR).

44. The method of claim 29 wherein hypomethylation is detected by detecting increased protein encoded by the gene.

45. The method of claim 29 wherein hypomethylation is detected by contacting at least a portion of the gene with a methylation-sensitive restriction endonuclease, said endonuclease preferentially cleaving non-methylated recognition sites relative to methylated recognition sites, whereby cleavage of the portion of the gene indicates non-methylation of the portion of the gene provided that the gene comprises a recognition site for the methylation-sensitive restriction endonuclease.

46. The method of claim 29 wherein hypomethylation is detected by contacting at least a portion of the gene of the cell with a chemical reagent that selectively modifies a non-methylated cytosine residue relative to a methylated cytosine residue, or selectively modifies a methylated cytosine residue relative to a non-methylated cytosine residue; and detecting a product generated by the contacting step.

47. The method of claim 46 wherein the step of detecting comprises hybridization with at least one probe that hybridizes to a sequence comprising a modified non-methylated CpG dinucleotide motif but not to a sequence comprising an unmodified methylated CpG dinucleotide.

48. The method of claim 46 wherein the step of detecting comprises amplification with at least one primer that hybridizes to a sequence comprising a modified non-methylated CpG dinucleotide motif but not to a sequence comprising an unmodified methylated CpG dinucleotide motif thereby forming amplification products.

49. The method of claim 46 wherein the step of detecting comprises amplification with at least one primer that hybridizes to a sequence comprising an unmodified methylated CpG dinucleotide motif but not to a sequence comprising a modified non-methylated CpG dinucleotide motif thereby forming amplification products.

50. The method of claim 46 wherein the product is detected by a method selected from the group consisting of electrophoresis, hybridization, amplification, primer extension, sequencing, ligase chain reaction, chromatography, mass spectrometry, and combinations thereof.

51. The method of claim 46 wherein the chemical reagent is hydrazine.

52. The method of claim 51 further comprising cleaving the hydrazine-contacted at least a portion of the gene with piperidine.

53. The method of claim 46 wherein the chemical reagent comprises bisulfite ions.

54. The method of claim 53 further comprising treating the bisulfite ion-contacted at least a portion of the gene with alkali.

55. A method of determining the prognosis of a subject having a cell proliferative disorder comprising:
determining the methylation state of at least one regulatory region of a gene in a nucleic acid sample from the subject, wherein hypomethylation as compared to a corresponding normal cell in the subject or a subject not having the disorder, is indicative of a poor prognosis.

56. (canceled)

57. (canceled)

58. (canceled)

59. (canceled)

60. (canceled)

61. (canceled)

62. (canceled)

63. (canceled)

64. (canceled)

65. A method of identifying a gene activated by hypomethylation comprising:
comparing an expression analysis of a cell treated with an agent that reduces methylation to an expression analysis of a control cell not treated with the agent, wherein an increase in expression of a gene is indicative of a gene activated by demethylation.

66. The method of claim 65, wherein the cell is a minimally transformed cell line.

67. The method of claim 65, wherein the demethylating agent is 5-aza-deoxycytidine.

68. The method of claim 65, further comprising an expression analysis of a tissue sample and a tumor sample from the same tissue of origin as the normal cell, wherein an increase in expression of a gene in a tumor sample as compared to a normal sample is correlated to the genes activated by demethylation in the treated cell.

69. The method of claim 68, further comprising sequence analysis of the identified genes to confirm the presence of CpG islands in the promoter region of the genes.

70. The method of claim 69, further comprising determining the methylation status of the promoter regions of the identified genes.

71. The method of claim 70, wherein the determining of the methylation status comprises contacting the gene with a chemical reagent that selectively modifies a non-methylated cytosine residue relative to a methylated cytosine residue, or selectively modifies a methylated cytosine residue relative to a non-methylated cytosine residue; and detecting a product generated by the contacting step.

72. The method of claim 71 wherein the chemical reagent comprises bisulfite ions.

73. The method of claim 72 further comprising treating the bisulfite ion-contacted at least a portion of the gene with alkali.

74. A method for determining whether a subject is responsive to a particular therapeutic regimen comprising determining the methylation status of one or more genes or regulatory regions thereof, selected from the group consisting of TKTL1, H19, MAGEA2, MAGEA3, MAGEA4, MAGEA11, GPR17, GRIN1, C19ORF28, MAGEA12, MAGEA1, MAGEA5, NY-ESO-1, MAGEA9, MAGEA6, MAGEB2, CT45-2, SBSN, G6PD, ZNF711, CrispL, KRT86, KIPV467, KRT81, CSPG5, PP1R14A, KISS1R, KIAA1937 protein, SOX30, DEAD, and KBGP wherein hypomethylation of the gene or regulatory region thereof as compared with a normal subject is indicative of a subject who is responsive to the therapeutic regimen.

75. (canceled)

76. (canceled)

77. (canceled)

78. A kit useful for the detection of a methylated CpG-containing nucleic acid in determining the methylation status of one or more genes or regulatory regions thereof, selected from the group consisting of TKTL1, H19, MAGEA2, MAGEA3, MAGEA4, MAGEA11, GPR17, GRIN1, C19ORF28, MAGEA12, MAGEA1, MAGEA5, NY-ESO-1, MAGEA9, MAGEA6, MAGEB2, CT45-2, SBSN, G6PD, ZNF711, CrispL, KRT86, KIPV467, KRT81, CSPG5, PP1R14A, KISS1R, KIAA1937 protein, SOX30, DEAD, and KBGP comprising: a carrier element containing one or more containers comprising a first container containing a reagent which modifies unmethylated cytosine and a second container containing primers for amplification of the one or more genes or regulatory regions thereof, wherein the primers distinguish between modified methylated and nonmethylated nucleic acid.

79. (canceled)
